(19) United States
(12) Patent Application Publication
(10) Pub. No.: US 2010/0120810 A1
(43) Pub. Date: May 13, 2010

(54) COMPOUNDS AND METHODS FOR MODULATING RHO GTPASES

(76) Inventors: Bertrand Leblond, Paris (FR); Eric Beausoleil, Paris (FR)

Correspondence Address: NIXON & VANDERHYE, PC, 901 NORTH GLEBE ROAD, 11TH FLOOR, ARLINGTON, VA, 22203, US

(21) Appl. No.: 12/452,535

(22) PCT Filed: Jul. 11, 2008
(86) PCT No.: PCT/EP2008/059134

§ 371 Date: Jan. 7, 2010

(30) Foreign Application Priority Data
Jul. 12, 2007 (EP) 07301230.4

(51) Int. Cl. A61K 31/4738; C07D 217/20; C07D 471/04; C07D 491/14; A61P 7/00; A61P 9/00; A61P 25/00; A61P 35/00; A61K 31/472

(52) U.S. Cl. 514/280; 514/307; 546/48; 546/47; 546/149

(57) ABSTRACT

The present invention relates to methods and compositions that affect the GTP-binding activity of members of the Rho family GTPases, preferably Rac GTPases (Rac1, Rac1b, Rac2 and/or Rac3).

The present invention relates to methods and compositions that affect the GTP-binding activity of members of the Rho family GTPases, preferably Rac GTPases (Rac1, Rac1b, Rac2 and/or Rac3).

Rho family GTPases are molecular switches that control signalling pathways regulating cytoskeleton reorganization, gene expression, cell cycle progression, cell survival, and other cellular processes (Etienne-Manneville S., and Hall A., 2002, Nature, 420, 629-635, which is incorporated herein by reference in its entirety).

Rho GTPases of the Ras superfamily are involved in the regulation of multiple cell functions and have been implicated in the pathology of various human diseases including cancers (Fritz G., Just I., and Kaina B., Int. J. Cancer, 1999, 81, 682-687; Fritz G., Kaina B. Curr. Cancer Drug Targets, 2006, 6, 1-14; Sahai E., Marshall C. J., Nat. Rev. Cancer., 2002, 2, 133-42), pathological angiogenesis such as in diabetic retinopathy, tumoral angiogenesis, glaucoma, age-related macular degeneration (Eriksson A., Cao R., Roy J., Tritsaris K., Wahlestedt C., Dissing S., Thyberg J., Cao Y., Circulation, 2003, 107, 1532-8; Soga N., Namba N., McAllister S., Cornelius L., Teitelbaum S. L., Dowdy S. F., Kawamura J., Hruska K. A., Exp. Cell. Res., 2001, 269, 73-87; Fryer B. H., Field J., Cancer Lett., 2005, 229, 13-23), asthma, Alzheimer's disease (Désiré L., Bourdin J., Loiseau N., Peillon H., Picard V., De Oliveira C., Bachelot F., Leblond B., Taverne T., Beausoleil E., Lacombe S., Drouin D., Schweighoffer F., J. Biol. Chem., 2005, 280(45), 37516-25), cardiac left ventricular hypertrophy (Brown J. H., Del Re D. P., Sussman M. A., Circ Res., 2006, 98, 730-42; Molkentin J. D., Dorn II G. W., 2^{nd} Annu Rev Physiol. 2001, 63, 391-426). They are attractive drug targets in future targeted therapy (Nassar N., Cancelas J., Zheng J., Williams D. A., and Zheng Yi, Current topics in Medicinal Chemistry, 2006, 6, 1109-1116).

Rho family proteins constitute one of three major branches of the Ras superfamily. Rho proteins share approximately 30 percent amino acid identity with the Ras superfamily proteins. At least 14 mammalian Rho family proteins have been identified so far, including RhoA, RhoB, RhoC, RhoE/Rnd3, Rnd1/Rho6, Rnd2/Rho7, RhoG, Rac1, Rac1b, Rac2, Rac3, Cdc42, TC10, and TTF.

Rac proteins (Rac1, 1b, 2, 3) belong to the Rho GTP-binding proteins (or GTPases) of the Ras superfamily and thus act as molecular switches cycling between an active GTP-bound and an inactive GDP-bound form through nucleotide exchange and hydrolysis. Like most other GTPases, these proteins adopt different conformations depending on the bound nucleotide, the main differences lying in the conformation of two short and flexible loop structures designated as the switch I and switch II regions. The three distinct mammalian Rac isoforms, Rac 1, 2 and 3, share a very high sequence identity (up to 90%), with Rac1b being an alternative splice variant of Rac1 with a 19 amino acid insertion in vicinity to the switch II region. Rac1b has an accelerated GEF-independent GDP/GTP-exchange and an impaired GTP-hydrolysis, accounting for a self-activating GTPase (Haeusler L. C. et al., Methods in Enzymology, 2006, 406, 1-11).

Rac1 regulates the activity of the superoxide anion generating NADPH oxidase system of phagocytes, plays a central role in organization of the actin cytoskeleton, and is essential for Ras-induced transformation. In addition, mutant, constitutively active Rac1b can induce cellular transformation, invasion, and metastasis. Similar to Ras proteins, Rac1 is activated by upstream GEFs (Guanine nucleotide Exchange Factors) and binds effector proteins that signal downstream. Human cells contain 3 homologous Rac proteins, Rac1, Rac2, and Rac3, that are essentially identical except for the hypervariable C-terminal domains. Rac1, but not Rac2 or Rac3, contains a polybasic domain within its hypervariable region that is virtually identical to the polybasic domain of K-Ras 4B.

Rac1 binds to and activates the effector protein PAK1 far more efficiently than Rac2 does, and the polybasic domain of Rac1 directly accounts for the enhanced ability of Rac1 to bind to and activate PAK1 (Knaus U. G., Wang Y., Reilly A. M., Warnock D., and Jackson J. H., J. Biol. Chem., 1998, 273, 21512). The polybasic domain is also crucial for Rac1 mediated activation of NADPH oxidase and membrane ruffling but is not required for Rac1 mediated cell transformation or binding of Rac1 to the effector protein POR1 (Jones M. K., and Jackson J. H., J. Biol. Chem., 1998, 273, 1782).

NSC 23766 described in international patent application WO 2007/016539 is a cell-permeable pyrimidine compound that specifically and reversibly inhibits Rac1 GDP/GTP exchange activity by interfering Rac1 interaction with the Rac-specific GEFs (guanine nucleotide exchange factor) Trio and Tiam1 (IC₅₀˜50 μM). NSC 23766 inhibit Rac1-mediated cellular functions in NIH3T3 and PC-3 cells (effective dose ˜50 to 100 μM). NSC 23766 exhibits no effect on Cdc42 or RhoA activation, nor does it affect Rac1 interaction with BcrGAP or PAK1 (Nassar N., Cancelas J., Zheng J., D. Williams A., and Zheng Yi, Current topics in Medicinal Chemistry, 2006, 6, 1109-1116).

EHT 1864 described in international patent application WO 2004/076445, is a small molecule that blocks the Rac1 signaling pathways. In vitro, EHT 1864 blocks Abeta 40 and Abeta 42 production but does not impact sAPPalpha levels and does not inhibit beta-secretase. Rather, EHT 1864 modulates APP processing at the level of gamma-secretase to prevent Abeta 40 and Abeta 42 generation. This effect does not result from a direct inhibition of the gamma-secretase activity and is specific for APP cleavage, since EHT 1864 does not affect Notch cleavage. In vivo, EHT 1864 significantly reduces Abeta 40 and Abeta 42 levels in guinea pig brains at a threshold that is compatible with delaying plaque accumulation and/or clearing the existing plaque in brain. EHT 1864 was the first derivative of a new chemical series that consists of candidates for inhibiting Abeta formation in the brain of Alzheimer patients as described in US patent No. 2007/0027146 (compound 38). EHT 1864 represented the first pharmacological validation of Rac1 signaling as a target for developing novel therapies for Alzheimer's disease (Désiré L., Bourdin J., Loiseau N., Peillon H., Picard V., De Oliveira C., Bachelot F., Leblond B., Taverne T., Beausoleil E., Lacombe S., Drouin D., and Schweighoffer F., J. Biol. Chem., 280 (45), 2005, 37516-25).

Berberine is a member of the protoberberine class of isoquinoline alkaloids. It is probably the most widely distributed of all alkaloids, having been found in the roots, rhizomes, and stem bark of the plants of nine botanical families, Berberidaceae, Papaveraceae, Rununculaceae, Rutaceae, Menispermaceae, Rubiaceae, Rhamnaceae, Magnoliaceae, and Annonaceae.

Other known members of the protoberberine class of isoquinoline alkaloids are jatrorrhizine chloride, columbamine chloride, berberrubine chloride, thalifendine chloride, coptisine chloride and nandinine hydrochloride, etc. . . . .

The protoberberine alkaloids display a broad diversity of biological activities (Simeon S., Rios J. L., and Villar A., Plant Med. Phytother., 1989, 23, 202; Bhakuni D. A., and Jain S., The Alkaloids, Academic Press, 1986, 28, 95; Shamma M., and Moniot J. L., Isoquinoline Alkaloids Research 1972-1977, Plenum Press, New York & London, 1978, 209; Shamma M., The Isoquinoline Alkaloids, Chemistry and Pharmacology, Academic Press, New York & London, 1972, 268; Kondo Y., Heterocycles, 1976, 4, 197) and feature predominently as active components in many folkloric medicines (Thakur, R. S., Srivastava, S. K., Cent. Inst. Med. Aromatic Plants, 1982, 4, 249) especially in Native America, China and other Asian countries.

In traditional practices of Ayurvedic and Chinese medicine, numerous plants have been used to treat cognitive disorders, including neurodegenerative diseases such as Alzheimer's disease (AD). Coptis chinensis (Rununculaceae) has been used in traditional Chinese medicine for several conditions. A methanol extract fraction of C. chinensis, jatrorrhizine and berberine are MAO inhibitors (Kong L. D., Cheng, C. H. and Tan R. X., Planta Med., 2001, 67(1), 74-76), indicating potential antidepressant activity, and C. chinensis and some alkaloids isolated from this plant (berberine, coptisine and palmatine) are reported to be anti-ChE (Huang K. C., CRC Press, Boca Raton (Fla.) 1993; Park C. H., Kim S., Choi W., Lee Y., Kim J., Kang S. S. et al., Planta Med., 1996, 62, 405-409 and Shigeta K., Ootaki K., Tatemoto H., Nakanishi T., Inada A., and Muto, N., Biosci. Biotechnol. Biochem., 2002, 66(11), 2491-2494). C. chinensis has also shown anti-inflammatory (Cuéllar M. J., Giner R. M., Recio M. C., Máñez S., and Ríos J. L., Fitoterapia, 2001, 72(3), 221-229) and antioxidant activities (Liu F., and Ng T. B., Life Sci., 2000, 66(8), 725-735 and Schinella G. R., Tournier H. A., Prieto J. M. Mordujovich de Buschiazzo P., and Ríos J. L., Life Sci., 2002, 70, 1023-1033) and it improved a scopolamine-induced learning and memory deficit in rats (Hsieh M. T., Peng W. H., Wu C. R., and Wang W. H., Phytother. Res., 2000., 14(5), 375-377). As well as inhibiting AChE, the alkaloids coptisine, palmatine and berberine in particular, also showed NGF-enhancing activity in PC12 cells (Shigeta K., Ootaki K., Tatemoto H., Nakanishi T., Inada A., and Muto N., Biosci. Biotechnol. Biochem., 2002, 66(11), 2491-2494).

Other pharmacological properties include antimicrobial, antimalarial, antileukemic, antiulcerous, gastric antisecretory, and enzyme inhibitory activities.

The mechanism of antimicrobial activity of berberine is related to its effect on DNA intercalation and inhibition of reverse transcription and DNA synthesis in microorganism cells. Berberine has an antimicrobial activity against a variety of organisms including bacteria, viruses, fungi, protozoans, helminths, and chlamydia. Currently, the predominant clinical uses of berberine include bacterial diarrhea, intestinal parasite infections and treatment of infected eyes and eye irritations (Murine™).

Berberine reduces total cholesterol, low-density lipoprotein (LDL) cholesterol, and triglycerides in both humans (at 1 g/day) and hamsters fed 50 mg/Kg/day along with a high fat diet. Berberine is therefore a natural product that may help control serum cholesterol without the side effects typical of the statin family of hypocholesterolemic drugs (Kong W., Wei J., Abidi P., et al., Nature Med., 2004, 10(12), 1344-1351).

The cytotoxicity of several protoberberine alkaloids against human cancer cell line (lung, colon, CNS, stomach, ovarian, breast, renal, melanoma) was also investigated (Iwasa K., Moriyasu M., Yamori T., Turuo T., Lee D.-U., and Wiegrebe W., J. Nat. Prod., 2001, 64, 896-898). It was shown that the cytoxic activity paralleled the antimicrobial activity.

Coralyne has more pronounced antitumor activity relative to berberine, exhibiting significant activity in vivo in mice against L1210 and P388 leukemias (Zee-Cheng et al., J. Med. Chem., 1974, 17, 347). Structure activity studies have suggested that the presence of the methyl substituent at the 8-position and unsaturation at the 5,6-position of coralyne are strongly associated with the antitumor activity against L1210 and P388 leukemias.

3-Arylisoquinoline derivatives and their N-methylated analogs may be regarded as ring-opened analogs lacking C5-C6 moiety of coralyne or berberine.

A very limited number of 3-arylisoquinoline analogs, more precisely 1-phenyl-3-phenylisoquinoline analogs of coralyne and their N-methylated quaternized analogs were described in PCT/US/061676. 3-arylisoquinolines were synthesized and tested as topoisomerase inhibitor I and II but did not exhibit any significant topoisomerase poisoning activity. Structural rigidity was found as a critical requirement for retention of activity as topoisomerase poisons.

Sources of benzo[c]phenanthridine alkaloids include five plant families: Papaveraceae, Fumariaceae, Rutaceae, Capitofoliaceace and Meliaceae. The most important source of benzo[c]phenanthridine alkaloids are found in the Papaveraceae plant family. Sanguinarine and chelerythrine are quarternary alkaloids isolated respectively from the root of Sanguinaria canadensis L. and Chelidonium majus L. These alkaloids are known as sanguinaria extracts. Patents describing an extract of these alkaloids include USSR Pat. No. 495,311 and German Pat. No. 2,856,577. The benzo-[c]-phenanthridine alkaloids have valuable properties as antimicrobials as well as in treating mouth odors, gingivitis, and periodontitis. Extract of the plant has been used in toothpastes and oral rinse products (Kufrinec M. M., Mueller-Joseph L. J., Kopczyk R. A., J. Can. Dent. Assoc., 1990, 56, 31-35). Such alkaloids can be purchased commercially and/or isolated from plants as known in the art and as described, for example, in U.S. Pat. No. 5,133,981.

Other known members of the benzo[c]phenanthridine alkaloids are fagaronine, nitidine, oxysanguinarine, oxyavycine, oxynitidine, norsanguinarine, chelirubine, macarpine, 6-oxochelerythrine, 5,6-dihydrochelerythrine, norchelerythrine, etc. . . . .

Benzo[c]phenanthridine alkaloids are known to have anticancer properties. (Stermitz F. R., Gillespie J. P., Amoros L. G., Romero R., Stermitz T. A., Larson K. A., Earl S., and Ogg J. E., J. Med. Chem., 1975, 18(7), 708-713).

Changes in activation balance of different protein kinase C (PKC) isoenzymes have been linked to cancer development. Interestingly, sanguinarine was shown essentially inactive against PKC (217 μm) (Wang, B. H., Lu, Z. X., and Polya, G. M., Planta Med. 1997, 63, 494-498), whereas the closely related chelerythrine has been reported as a potent (1 μm) inhibitor of this kinase. Mitogen-activated protein kinase phosphatase-1 (MKP-1) is a dual specificity phosphatase that is overexpressed in many human tumors and can protect cells from apoptosis caused by DNA-damaging agents or cellular stress. Both chelerythrine and sanguinarine have MKP-1 inhibitory activity (Vogt A., Tamewitz A., Skoko J., Sikorski R. P., Giuliano K. A., and Lazo J. S., J. Biol. Chem., 2005, 280 (19), 19078-19086).

Sanguinarine is also known to possess interesting antiangiogenic properties (Giuseppina B. et al., Ann. N.Y. Acad. Sci., 2007, 1095, 371-376). This process impacts significantly on many important disease states including cancer, diabetic retinopathy, and arthritis.

Chelerythrine is also currently in development for the treatment of bipolar disorder and the cognitive deficits of schizophrenia. Chelerythrine's utility for treating CNS disorders, based on its PKC inhibition, was discovered by Amy Arnsten at Yale University (international patent application WO 2005/030143). Taken orally, chelerythrine has proven to be very potent in multiple models of memory disorders including a sophisticated primate model of prefrontal cortex-dependent working memory.

The present invention is based on the identification of the inhibitory action of particular protoberberine alkaloids, benzo[c]phenanthridines alkaloids and 3-arylisoquinolines (ring-opened analogs of coralyne) on the activity of Rho family GTPases, in particular on the activity of the members of Rac subfamily of Rho GTPases.

Accordingly, the present invention relates to the use of protoberberine, benzo[c]phenanthridine alkaloids or 3-arylisoquinolines derivatives in an in vitro method for modulating, preferably inhibiting, a member of the Rho GTPase family.

The present invention further relates to the use of a compound of formula (I) or (II) as defined herein below for the manufacture of a pharmaceutical composition for treating a pathology involving a member of the Rho GTPase family.

The invention further relates to compounds of formula (I), and in particular of compounds of formula (V), or of compounds of formula (II) as defined herein below and pharmaceutical compositions comprising the same.

It has been found that compounds of formula (I) and (II) described below, and pharmaceutically acceptable derivatives thereof, specifically inhibit Rho GTPases, in particular Rac GTPases, and can be effective in modulating Rho GTPases functions, in particular Rac-mediated functions, in diverse cellular systems including tumor cell transformation and invasion and hematopoietic stem/progenitor cell mobilization.

One object of the invention is thus to provide an in vitro method for inhibiting a member of the Rho GTPase family, wherein the GTPase is contacted with at least one compound of formula (I) or (II),

a compound of formula (I) having the following structure:

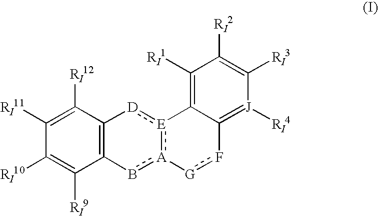

in which
J represents C or N;
R_{I}¹, R_{I}², R_{I}³ and R_{I}⁴ independently represent H, a halogen atom, a (C₁-C₆)alkyl group, an —OH group, an —O—(C₁-C₆)alkyl group, a (C₂-C₆)alkenyl group, a (C₂-C₆)alkynyl group, a —NO₂ group, a —NH₂ group, a —CO—(C₁-C₆)alkyl group preferably a —COCH₃ group, a —NH—SO₂—CH₃ group, a —N(SO₂CH₃)₂ group, a —NH—CO—CH₃ group, a NH—CO—N(CH₃)₂ group, a —COOH group, a —COO(C₁-C₆)alkyl group preferably a —CO—O—CH(CH₃)₂ group, or a —CONH(C₁-C₆)alkyl group preferably a —CONHCH₃ group,
R_{I}⁴ being absent when J represents N and R_{I}⁴ being present when J represents C;
R_{I}⁹, R_{I}¹⁰ and R_{I}¹¹ independently represent H, an —OH group or an —O—(C₁-C₆)alkyl group;
or alternatively R_{I}² and R_{I}³ and/or R_{I}³ and R_{I}⁴ are fused together so as to form a naphthalene group or a quinolyl group with the adjacent cycle, or an —O—(CH₂)—O— group linked to the adjacent cycle, wherein n is an integer comprised between 1 and 6, and/or R_{I}⁹ and R_{I}¹⁰ and/or R_{I}¹⁰ and R_{I}¹¹ are fused together so as to form an —O—(CH₂)—O— group linked to the adjacent cycle, wherein n is an integer comprised between 1 and 6;
R_{I}¹² represents H, a (C₁-C₆)alkyl group, a (C₂-C₆)alkenyl group or a (C₂-C₆)alkynyl group;
A represents N, N⁺, NH, N⁺H, N—(C₁-C₆)alkyl, N⁺-(C₁-C₆)alkyl, N-arylalkyl preferably N-benzyl, or N⁺-arylalkyl preferably N⁺-benzyl;
B, absent or present, represents CH, CH₂, C-Methyl, C-Benzyl or C-Phenyl when B is present;
D, absent or present, represents CH or CH₂ when D is present;
E represents C, CH or CH₂;
G and F, absent or present, both represent either CH or CH₂ when present;
with the provisos that

at least one of B and D is present

both B and D are present when G and F are absent; and

when B or D is absent exclusively, then G and F are present;

its tautomers, optical and geometrical isomers, racemates, salts, hydrates and mixtures thereof;
and the compound of formula (II) having the following structure:

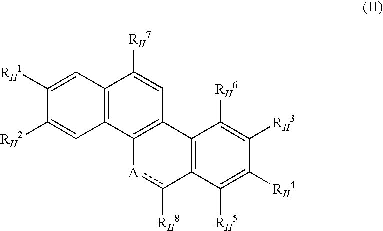

in which
R_{II}¹, R_{II}², R_{II}⁴ and R_{II}⁵ independently represent H, —OH or a —O—(C₁-C₆)alkyl group;
or alternatively wherein R_{II}¹ and R_{II}² and/or R_{II}⁴ and R_{II}⁵ are fused together so as to form an —O—(CH₂)—O— group linked to the adjacent cycle, wherein n is an integer comprised between 1 and 6;
R_{II}³, R_{II}⁶, R_{II}⁷ and R_{II}⁸ independently represent H, a (C₁-C₆)alkyl group, a (C₂-C₆)alkylene group or a (C₂-C₆)alkynyl group; and
A represents N, N⁺, N⁺-(C₁-C₆)alkyl or N⁺-benzyl;
its tautomers, optical and geometrical isomers, racemates, salts, hydrates and mixtures thereof.

When D is absent in formula (I), member B is present and both G and F are present. Compounds according to this definition are compounds of formula (III):

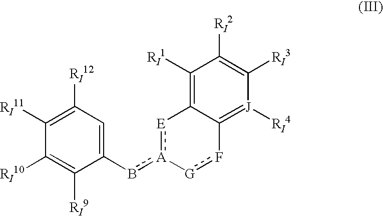

wherein R_{I}¹, R_{I}², R_{I}³, R_{I}⁴, R_{I}⁹, R_{I}¹⁰, R_{I}¹¹, R_{I}¹², A, B, E, F, G and J are as defined above.

Compounds of formula (III′) are compounds of formula (III) wherein J represents C:

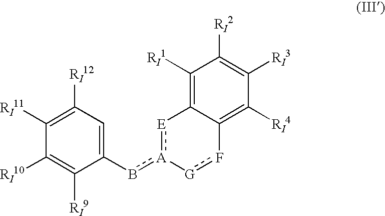

When B is absent in formula (I), member D is present and both G and F are present. Compounds according to this definition are compounds of formula (IV):

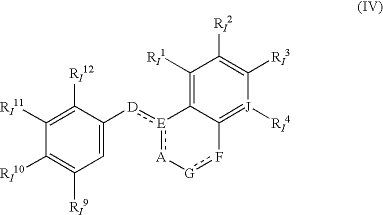

wherein R_{I}¹, R_{I}², R_{I}³, R_{I}⁴, R_{I}⁹, R_{I}¹⁰, R_{I}¹¹, R_{I}¹², A, B, E, F, G and J are as defined above.

Compounds of formula (IV′) are compounds of formula (IV) wherein J represents C:

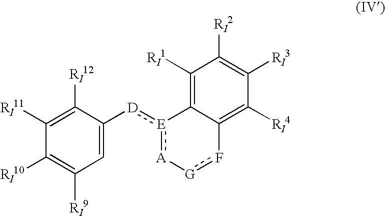

In the structures depicted above, a dotted line denotes the presence or not of a double bond at the indicated position.

When B represents C-Benzyl in formula (I), (IV) or (IV′), the carbone atom is linked to the methyl group of the benzyl moiety, as illustrated in the following structure:

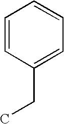

Similarly, when A represents N⁺-benzyl in formula (I), (II), (III), (III′), (IV) or (IV′), the nitrogen atom is linked to the methyl group of the benzyl moiety, as illustrated in the following structure:

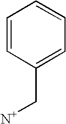

In the present application alkyl, alkenyl and alkynyl groups may be substituted or not by at least one substituent such as halo, amino, cyano, hydroxy, alkoxy, alkylthio, ˜NH(alkyl), —NH (cycloalkyl), —N(alkyl)₂, —C(═O)H, —CO₂H, —CO₂-alkyl, cycloalkyl, substituted cycloalkyl, aryl, heteroaryl, or heterocycle.

Within the context of the present application, the term alkyl denotes linear or branched saturated groups containing from 1 to 6 carbon atoms. Examples of alkyl groups having from 1 to 6 carbon atoms inclusive are methyl, ethyl, propyl, isopropyl, t-butyl, n-butyl, pentyl, hexyl, 2-methylbutyl, 2-methylpentyl and the other isomeric forms thereof. Preferably, the alkyl groups have from 1 to 3 carbon atoms.

The cycloalkyl group is more specifically an alkyl group forming at least one cycle. Examples of cycloalkyl groups having from 3 to 8 carbon atoms inclusive are cyclopropyl, cyclobutyl, cyclopentyl and cyclohexyl. The cycloalkyl group may be optionally substituted.

The term heterocycle is understood to refer to hydrocarbon cyclic group having from 1 to 20 carbon atoms, optionally interrupted with one or more heteroatoms selected in the group consisting of N, O, S and P. Among such mono- or poly-cyclic hydrocarbon groups, cyclopentyl, cyclohexyl, cycloheptyl, 1- or 2-adamantyl groups, pyran, piperidine, pyrrolidine, morpholine, dioxan, tetrahydrothiophene, and tetrahydrofuran can be cited.

The term alkenyl denotes linear or branched hydrocarbon groups containing from 2 to 6 carbon atoms and containing at least one double bond. Examples of alkenyl containing from 3 to 6 carbon atoms are 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl and the isomeric forms thereof.

The term alkynyl denotes linear or branched hydrocarbon groups containing from 2 to 6 carbon atoms and containing at least one triple bond. Examples of alkynyl containing from 3 to 6 carbon atoms are 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl and the isomeric forms thereof.

The term aryl includes any aromatic group comprising preferably from 5 to 14 carbon atoms, preferably from 6 to 14 carbon atoms, optionally interrupted by one or several heteroatoms selected from N, O, S or P (termed, more specifically, heteroaryl). Most preferred aryl groups are mono- or bi-cyclic and comprises from 6 to 14 carbon atoms, such as phenyl, α-naphtyl, β-naphtyl, antracenyl, or fluorenyl group.

An alkoxy group denotes an —O-alkyl group and an alkylthio group denotes an —S—alkyl group.

The term “halo” refers to fluorine, chlorine, bromine and iodine.

The in vitro method of the invention may be useful for different purposes. For example, a compound of formula (I) or (II) may be used to modulate, preferably to inhibit, the Rho GTPases, preferably Rac GTPases, in a cell culture for study of the signal pathways involving said GTPases and understanding their biochemical functions or those of their effectors.

In another example, one can use a compound of formula (I) or (II) for modulating, preferably inhibiting, in vitro a Rho GTPase, preferably a Rac GTPase, in a screening assay. More specifically, the invention relates to a method for identifying, selecting or characterizing compounds modulating in vitro a Rho GTPase, preferably a Rac GTPase, comprising contacting said GTPase with at least one compound of formula (I) or (II) as defined above and with a test compound, and measuring the activity of said GTPase. In a particular embodiment, the method for identifying, selecting or characterizing compounds modulating in vitro a Rho GTPase, preferably a Rac GTPase, further comprises comparing the activity of said GTPase in presence of the test compound to the activity of said GTPase in the absence of the test compound. More particularly, the activity of said GTPase can be measured as described below.

In a particular embodiment of the method of the invention, the member of the Rho GTPase family is contacted with a compound of formula (I′), corresponding to formula (I) in which J represents C:

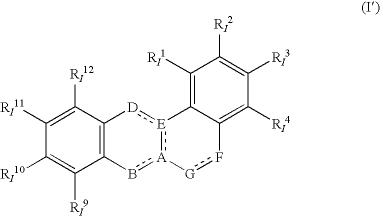

and in which
R_{I}¹, R_{I}⁴ and R_{I}¹² independently represent H, a (C₁-C₆)alkyl group, a (C₂-C₆)alkenyl group or a (C₂-C₆)alkynyl group;
R_{I}², R_{I}³, R_{I}⁹, R_{I}¹⁰ and R_{I}¹¹ independently represent H, —OH or an —O—(C₁-C₆)alkyl group;
or alternatively R_{I}² and R_{I}³ and/or R_{I}⁹ and R_{I}¹⁰ and/or R_{I}¹⁰ and R_{I}¹¹ are fused together so as to form an —O—(CH₂)ₙ—O— group linked to the adjacent cycle, wherein n is an integer comprised between 1 and 6;
A represents N, N⁺, N⁺-(C₁-C₆)alkyl or N⁺-benzyl;
B, absent or present, B representing CH, CH₂, C-methyl, C-Benzyl or C-Phenyl when present;
D, absent or present, D representing CH or CH₂ when present;
with the proviso that at least one of B and D is present;
E represents C, CH or CH₂; and
F and G both represent either CH or CH₂;
its tautomers, optical and geometrical isomers, racemates, salts, hydrates and mixtures thereof.

In a particular embodiment of the method of the invention, the member of the Rho GTPase family is contacted with a compound of formula (I) or (I′) in which R_{I}² and/or R_{I}³ represent —OH.

In another particular embodiment of the method of the invention, the member of the Rho GTPase family is contacted with a compound of formula (I) or (I′) wherein R_{I}² and R_{I}³ and/or R_{I}⁹ and R_{I}¹⁰ and/or R_{I}¹⁰ and R_{I}¹¹ are fused together so as to form an —O—(CH₂)—O— group linked to the adjacent cycle and preferably wherein n is 1.

In a further particular embodiment of the method of the invention, the member of the Rho GTPase family is contacted with a compound of formula (II), wherein R_{II}¹ and R_{II}² and/or R_{II}⁴ and R_{II}⁵ are fused together so as to form an —O—(CH₂)ₙ—O— group linked to the adjacent cycle and preferably wherein n is 1.

In a particular embodiment of the method of the invention, the member of the Rho GTPase family is contacted with a compound of formula (I) or (I′) wherein at least one of R_{I}², R_{I}³, R_{I}⁹, R_{I}¹⁰ and R_{I}¹¹ groups represents an —O—(C₁-C₆)alkyl group, preferably an —O-methyl group.

In another particular embodiment of the method of the invention, the member of the Rho GTPase family is contacted with a compound of formula (II) wherein at least one of R_{II}¹, R_{II}², R_{II}⁴ and R_{II}⁵ groups represents an —O—(C₁-C₆)alkyl group, preferably an —O-methyl group.

In a further embodiment of the method of the invention, the member of the Rho GTPase family is contacted with a compound of formula (I) or (I′) wherein R_{I}¹, R_{I}⁴ and/or R_{I}¹² represent H, preferably wherein R_{I}¹ and R_{I}⁴ represent H and/or R_{I}¹² represents H and

In another particular embodiment of the method of the invention, the member of the Rho GTPase family is contacted with a compound of formula (I) or (I′) wherein R_{I}¹⁰ represent an —O-methyl or —OH group and optionally R_{I}⁹, or alternatively R_{I}¹¹, represents an —O-methyl or —OH group. In a further embodiment, R_{I}¹⁰ and R_{I}⁹, or alternatively R_{I}¹⁰ and R_{I}¹¹, are the same and preferably represent either an —O-methyl or —OH group.

In another particular embodiment of the method of the invention, the member of the Rho GTPase family is contacted with a compound of formula (I) or (I′) in which R_{I}², R_{I}³, R_{I}⁹ and/or R_{I}¹⁰ represent —OH.

In a further embodiment of the method of the invention, the member of the Rho GTPase family is contacted with a compound of formula (I) or (I′) in which R_{I}² and R_{I}³ are —OH and/or R_{I}⁹ and R_{I}¹⁰ are —OH.

In another particular embodiment of the method of the invention, the member of the Rho GTPase family is contacted with a compound of formula (I) or (I′) in which R_{I}², R_{I}³, R_{I}⁹ and R_{I}¹⁰ represent —OH.

In a particular embodiment of the method of the invention, the member of the Rho GTPase family is contacted with compounds of formula (I) or (I′) in which R_{I}² and R_{I}³ represent —OH, A is N⁺, B and D represent CH, E represents C and F and G simultaneously represent CH₂.

In a particular embodiment of the invention, when A in the compound of formula (I), (I′) or (II) represents N⁺, N⁺-(C₁-C₆)alkyl or N⁺-benzyl, said compound of formula (I), (I′) or (II) is an ammonium salt in complex with any suitable counter ion. For example, such compound may be a halide salt such as bromide, chloride, fluoride or iodide salt or an acetate (CH₃—COO⁻) salt.

In a particular embodiment of the invention, the member of the Rho GTPase family is contacted with a compound of formula (I) wherein G and F are absent.

In a further particular embodiment of the invention, the member of the Rho GTPase family is contacted with a compound of formula (I) wherein G and F are absent and R_{I}¹, R_{I}², R_{I}³ and R_{I}⁴ represent H. In this embodiment, A may further represent N or N⁺-Methyl and/or R_{I}⁹, R_{I}¹⁰ and/or R_{I}¹¹, preferably R_{I}¹⁰ and R_{I}¹¹ represent an —OH group or an —O—(C₁-C₆)alkyl group, preferably an —O—CH₃ group.

In another particular embodiment of the invention, the member of the Rho GTPase family is contacted with a compound of formula (I) wherein G and F are absent and R_{I}¹⁰ and R_{I}¹¹, which are the same or different, represent an —OH group or an —O—(C₁-C₆)alkyl group.

In a particular embodiment of the method of the invention, the member of the Rho GTPase family is contacted with a compound of formula (V), corresponding to formula (I) in which G and F are absent:

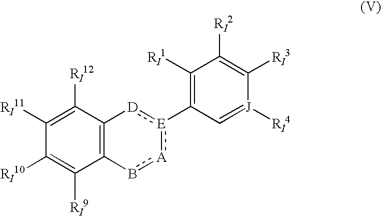

and in which
J represents C or N;
R_{I}¹ represents H, a halogen atom, a (C₁-C₆)alkyl group, an —O—(C₁-C₆)alkyl group, a (C₂-C₆)alkenyl group, a (C₂-C₆)alkynyl group, a —NO₂ group, a —NH₂ group, a —CO—(C₁-C₆)alkyl group preferably a —COCH₃ group, a —NH—SO₂—CH₃ group, a —N(SO₂CH₃)₂ group, a —NH—CO—CH₃ group, a —NH—CO—N(CH₃)₂ group, a —COOH group, a —COO(C₁-C₆)alkyl group preferably a —CO—O—CH(CH₃)₂ group, or a —CONH(C₁-C₆)alkyl group preferably a —CONHCH₃ group;
R_{I}², R_{I}³ and R_{I}⁴ independently represent H, a halogen atom, a (C₁-C₆)alkyl group, an —OH group, an —O—(C₁-C₆)alkyl group, a (C₂-C₆)alkenyl group, a (C₂-C₆)alkynyl group, a —NO₂ group, a —NH₂ group, a —CO—(C₁-C₆)alkyl group preferably a —COCH₃ group, a —NH—SO₂—CH₃ group, a —N(SO₂CH₃)₂ group, a —NH—CO—CH₃ group, a NH—CO—N(CH₃)₂ group, a —COOH group, a —COO(C₁-C₆)alkyl group preferably a —CO—O—CH(CH₃)₂ group, a —CONH(C₁-C₆)alkyl group preferably a —CONHCH₃ group;
R_{I}⁴ being absent when J represents N and R_{I}⁴ being present when J represents C;
R_{I}⁹, R_{I}¹⁰ and R_{I}¹¹ independently represent H or an —O—(C₁-C₆)alkyl group;
or alternatively R_{I}² and R_{I}³ or R_{I}³ and R_{I}⁴ are fused together so as to form a naphthalene group or a quinolyl group with the adjacent cycle, and/or R_{I}⁹ and R_{I}¹⁰ and/or R_{I}¹⁰ and R_{I}¹¹ are fused together so as to form an —O—(CH₂)ₙ—O— group linked to the adjacent cycle, wherein n is an integer comprised between 1 and 6;
R_{I}¹² represents H, a (C₁-C₆)alkyl group, a (C₂-C₆)alkenyl group or a (C₂-C₆)alkynyl group
A represents N, N⁺, NH, N⁺H, N—(C₁-C₆)alkyl, N⁺—(C₁-C₆)alkyl, N-arylalkyl preferably N-benzyl or N⁺-arylalkyl preferably N⁺-benzyl;
B represents CH, CH₂, C-Methyl, C-Benzyl or C-Phenyl;
D represents CH or CH₂;
E represents C or CH;
at least one of R_{I}¹, R_{I}², R_{I}³ and R_{I}⁴ being different from a hydrogen atom when J represents C;
with the proviso that if one of R_{I}², R_{I}³ and R_{I}⁴ represents a —O(C₁-C₆)alkyl group, the other ones of R_{I}², R_{I}³ and R_{I}⁴ do not represent a —O(C₁-C₆)alkyl group;
its tautomers, optical and geometrical isomers, racemates, salts, hydrates and mixtures thereof.

Particular embodiments of the method of the invention comprise the following embodiments (i)-(xvi). Each embodiments (i)-(xvi) and any combination of embodiments (i)-(xvi) is intended to be part of the disclosure of the present invention. Accordingly, for example, the present disclosure comprises combination of embodiments (i) and (ii), (i) and (ii) and (iii), (iii) and (iv), etc.

In embodiment (i), the method of the invention comprises contacting the member of the Rho GTPase family with a compound of formula (V) wherein J represents C.

In embodiment (ii), the method of the invention comprises contacting the member of the Rho GTPase family with a compound of formula (V) wherein two of R_{I}⁹, R_{I}¹⁰ and R_{I}¹¹ represent a —O(C₁-C₆)alkyl group, preferably a —O—CH₃ group and the other one represents H.

Preferably, in this embodiment, R_{I}¹⁰ and R_{I}¹¹ both represent a —O(C₁-C₆)alkyl group, preferably a —O—CH₃ group, and R_{I}⁹ represents H.

In embodiment (iii), the method of the invention comprises contacting the member of the Rho GTPase family with a compound of formula (V) wherein at least one of R_{I}¹, R_{I}², R_{I}³ and R_{I}⁴ represents a —NO₂ group, a —NH₂ group, a —NH—SO₂—CH₃ group, a —N(SO₂CH₃)₂ group, a —NH—CO—CH₃ group or a NH—CO—N(CH₃)₂ group.

In embodiment (iv), the method of the invention comprises contacting the member of the Rho GTPase family with a compound of formula (V) wherein at least one of R_{I}¹, R_{I}², R_{I}³ and R_{I}⁴ represents a —CO—(C₁-C₆)alkyl group, preferably a —COCH₃, a —COOH group, a —COO(C₁-C₆)alkyl group, preferably a —COOCH(CH₃)₂ group, or a —CONH(C₁-C₆)alkyl group, preferably a —CONHCH₃ group.

In embodiment (v), the method of the invention comprises contacting the member of the Rho GTPase family with a compound of formula (V) wherein at least one of R_{I}¹, R_{I}², R_{I}³ and R_{I}⁴ represents an —OH group, an —O—(C₁-C₆)alkyl group or a (C₁-C₆)alkyl group.

In embodiment (vi), the method of the invention comprises contacting the member of the Rho GTPase family with a compound of formula (V) wherein J represents C, R_{I}⁴ represents a hydrogen atom and R_{I}² and R_{I}³ are fused together so as to form a naphthalene group.

In embodiment (vii), the method of the invention comprises contacting the member of the Rho GTPase family with a compound of formula (V) wherein at least one of R_{I}¹, R_{I}², R_{I}³ and R_{I}⁴ represents a halogen atom.

In embodiment (viii), the method of the invention comprises contacting the member of the Rho GTPase family with a compound of formula (V) wherein one of R_{I}⁹, R_{I}¹⁰ and R_{I}¹¹ represents a hydrogen atom and the others, which are the same or different, preferably the same, represent —O(C₁-C₆)alkyl, preferably —OCH₃.

In embodiment (ix), the method of the invention comprises contacting the member of the Rho GTPase family with a compound of formula (V) wherein R_{I}¹ represents a hydrogen atom.

In embodiment (x), the method of the invention comprises contacting the member of the Rho GTPase family with a compound of formula (V) wherein R_{I}⁹ represents a hydrogen atom.

In embodiment (xi), the method of the invention comprises contacting the member of the Rho GTPase family with a compound of formula (V) wherein R_{I}¹² represents a hydrogen atom.

In embodiment (xii), the method of the invention comprises contacting the member of the Rho GTPase family with a compound of formula (V) wherein B represents C-Methyl or CH, preferably C-Methyl. Preferably, in this embodiment, R_{I}⁹ represents H and R_{I}¹⁰ and R_{I}¹¹ both represent an —O(C₁-C₆)alkyl group, preferably an —OCH₃ group.

In embodiment (xiii), the method of the invention comprises contacting the member of the Rho GTPase family with a compound of formula (V) wherein R_{I}¹, R_{I}⁹ and R_{I}¹² represent H.

In embodiment (xiv), the method of the invention comprises contacting the member of the Rho GTPase family with a compound of formula (V) wherein only one from R_{I}¹, R_{I}², R_{I}³ and R_{I}⁴ is substituted with a group or atom different from H.

In embodiment (xv), the method of the invention comprises contacting the member of the Rho GTPase family with a compound of formula (V) wherein only two from R_{I}¹, R_{I}², R_{I}³ and R_{I}⁴ are substituted with a group or atom different from H. Preferably, the substituants are selected in the group consisting of a halogen atom, preferably a chlorine atom, an —OH group and an —O(C₁-C₆)alkyl group. Preferably, both substituants are the same.

In embodiment (xvi), the method of the invention comprises contacting the member of the Rho GTPase family with a compound of formula (V) wherein R_{I}¹, R_{I}⁴, R_{I}⁹ and R_{I}¹² represent H, B represents C-methyl, A represents N or N⁺-methyl, RI¹⁰ and RI¹¹ both represent an —O(C₁-C₆)alkyl group, preferably an —O—CH₃ group, and J represents C.

In a particular embodiment of the method of the invention, the member of the Rho GTPase family, preferably a member of the Rac GTPase family, is contacted with a compound of formula (I) or (II), including a compound of any particular embodiment disclosed above, which inhibits the activity of said member by at least 10%, preferably at least 20%, preferably at least 30%, preferably at least 50%, preferably at least 60%, preferably at least 80%, preferably at least 90% and more preferably at least 95% at a concentration of the compound of 50 μM, as determined in the biological assays disclosed below. More specifically, the activity of a member of the Rho GTPase family, preferably a member of the Rac GTPase family, and the effect of a compound of formula (I) or (II), including a compound of any particular embodiment disclosed above, on said GTPase may be determined using an analog of GTP, BODIPY-GTP. The fluorescence of BODIPY-GTP increases when it binds to small G proteins. This property may be used to assess the ability of a compound to modulate the nucleotide binding activity of a GTPase, in particular in a biochemical exchange assay. More particularly, the effect of a compound of formula (I) or (II), including a compound of any particular embodiment disclosed above, may be assessed by determining the binding of BODIPY-GTP to Rac1 activated by the DH/PH domain of Tiam1, to Rac1b or to Cdc42.

Specific examples of compounds of formula (I) or (II) which may be used in the above in vitro method include the following compounds:

### Protoberberine Class of Isoquinoline Alkaloids Formula (I)

Berberine or 1,2-dimethoxy-N-methyl-[1,3]benzodioxolo[5,6-c]phenanthridinium chloride 1,
palmatine chloride, hydrate 2,
(±)-canadine or (±)-tetrahydroberberine hydrochloride 3,
demethyleneberberine or 9,10-dimethoxy-5,6-dihydro-isoquino[3,2-a]isoquinolinylium-2,3-diol chloride 4,
(±)-N-benzyl canadinium or (±)-7-benzyl-9,10-dimethoxy-5,8,13,13a-tetrahydro-6H-[1,3]dioxolo[4,5-g]isoquino[3,2-a]isoquinolinylium bromide 5,
2,3,9,10-tetrahydroxyberberine or 5,6-dihydro-isoquino[3,2-a]isoquinolinylium-2,3,9,10-tetraol chloride 6,
2-(2,3-dimethoxybenzyl)-6,7-dimethoxy-1,2,3,4-tetrahydroisoquinoline hydrochloride 7,
coralyne or 8-methyl-2,3,10,11-tetramethoxydibenzo[a,g]quinolizinium chloride, hydrate 8,
papaverine or 1-(3,4-dimethoxybenzyl)-6,7-dimethoxyisoquinoline hydrochloride 9,
9,10-dimethoxy-8-phenyl-5,8-dihydro-2H-6H-[1,3]dioxolo[4,5-g]isoquino[3,2-a]isoquinoline 10,
8-benzyl-9,10-dimethoxy-5,8-dihydro-2H-6H-[1,3]dioxolo[4,5-g]isoquino[3,2-a]isoquinoline 11,
(±)-8-benzyl-9,10-dimethoxy-5,8,13,13a-tetrahydro-6H-[1,3]dioxolo[4,5-g]isoquino[3,2-a]isoquinoline hydrochloride 12,
8-methyl-isoquino[3,2-a]isoquinolinylium-2,3,10,11-tetraol chloride 15,
(±)-tetrahydroxytetrahydroberberine or (±)-5,8,13,13a-tetrahydro-6H-isoquino[3,2-a]isoquinolinylium-2,3,9,10-tetraol hydrochloride 16.
(±)-9,10-Dimethoxy-5,8,13,13a-tetrahydro-6H-isoquino[3,2-a]isoquinoline-2,3-diol hydrochloride 17,
2-(2,3-Dihydroxybenzyl)-6,7-dimethoxy-1,2,3,4-tetrahydroisoquinolinium chloride 18,
2-(2,3-Dihydroxybenzyl)-6,7-dihydroxy-1,2,3,4-tetrahydroisoquinolinium chloride 19,
(±)-3-(6-Ethylbenzo[d][1,3]dioxol-5-yl)-7,8-dimethoxy-2-methyl-1,2,3,4-tetrahydroisoquinolinylium chloride 20.

### 3-Arylisoquinolines (Ring-Opened Analogs Lacking C₅-C₆ Moiety of Coralyne) Formula (V)

3-(Benzo[d][1,3]dioxol-5-yl)-6,7-dimethoxy-1-methylisoquinoline hydrochloride 21,
3-(Benzo[d][1,3]dioxol-5-yl)-6,7-dimethoxy-1,2-dimethylisoquinolinium chloride 22,
6,7-Dimethoxy-1-methyl-3-(3-nitrophenyl)isoquinolinium chloride 23,
1-(3-(6,7-Dimethoxy-1-methylisoquinolin-3-yl)phenyl)ethanone hydrochloride 24,
3-(3-Acetylphenyl)-6,7-dimethoxy-1,2-dimethylisoquinolinium chloride 25,
4-(6,7-Dimethoxy-1-methylisoquinolin-3-yl)benzene-1,2-diol hydrochloride 26,
3-(3,4-Dihydroxyphenyl)-6,7-dimethoxy-1,2-dimethylisoquinolinium chloride 27,
3-(6,7-Dimethoxy-1-methylisoquinolin-3-yl)aniline dihydrochloride 28,
N-(3-(6,7-Dimethoxy-1-methylisoquinolin-3-yl)phenyl)-N-(methylsulfonyl)methanesulfonamide hydrochloride 29,
N-(3-(6,7-Dimethoxy-1-methylisoquinolin-3-yl)phenyl)methanesulfonamide hydrochloride 30,
N-(3-(6,7-Dimethoxy-1-methylisoquinolin-3-yl)phenyl)acetamide hydrochloride 31,
Isopropyl 4-(6,7-dimethoxy-1-methylisoquinolin-3-yl)benzoate hydrochloride 32,
4-(6,7-Dimethoxy-1-methylisoquinolin-3-yl)benzoic acid hydrochloride 33,
4-(6,7-Dimethoxy-1-methylisoquinolin-3-yl)-N-methylbenzamide 34,
6,7-Dimethoxy-3-(6-methoxypyridin-3-yl)-1-methylisoquinoline dimethanesulfonate 35,
6,7-Dimethoxy-1-methyl-3-(pyridin-3-yl)isoquinoline dihydrochloride 36,
2-(6,7-Dimethoxy-1-methylisoquinolin-3-yl)aniline dihydrochloride 37,
N-(2-(6,7-Dimethoxy-1-methylisoquinolin-3-yl)phenyl)acetamide 38,
3-(3,4-Dichlorophenyl)-6,7-dimethoxy-1-methylisoquinolinylium chloride 39,
6,7-Dimethoxy-3-(4-methoxyphenyl)-1-methylisoquinolinylium chloride 40,
6,7-Dimethoxy-1-methyl-3-(naphthalen-2-yl)isoquinolinylium chloride 41,
3-(4-Chlorophenyl)-6,7-dimethoxy-1-methylisoquinolinylium chloride 42,
6,7-Dimethoxy-1-methyl-3-p-tolylisoquinolinylium chloride 43,
6,7-Dimethoxy-1-methyl-3-phenylisoquinolinylium chloride 44,
3-(3,4-Dihydroxyphenyl)-6,7-dihydroxy-1,2-dimethylisoquinolinium chloride 45,
3-(2-(6,7-Dimethoxy-1-methylisoquinolin-3-yl)phenyl)-1,1-dimethylurea hydrochloride 46,
4-(6,7-Dimethoxy-1-methylisoquinolin-3-yl)-2-methoxyphenol hydrochloride 47,
4-(6,7-dimethoxy-1-methylisoquinolin-3-yl)benzene-1,2-diol hydrochloride 48,
6,7-Dimethoxy-3-phenylisoquinolinium chloride 49,
6,7-Dimethoxy-2-methyl-3-phenylisoquinolinium chloride 50.

### Benzo[c]Phenanthridine Alkaloids Formula (II)

Sanguinarine or 13-methyl-[1,3]benzodioxolo[5,6-c]-1,3-dioxolo[4,5-i]phenanthridinium chloride hydrate 13,
chelerythrine or 1,2-dimethoxy-N-methyl[1,3]benzodioxolo[5,6-c]phenanthridinium chloride 14.
2,3-Dihydroxy-7,8-dimethoxy-5-methylbenzo[c]phenanthridinium chloride 51,
2,3,7,8-Tetrahydroxy-5-methylbenzo[c]phenanthridinium chloride 52.

### Protoberberine Class of Isoquinoline Alkaloids

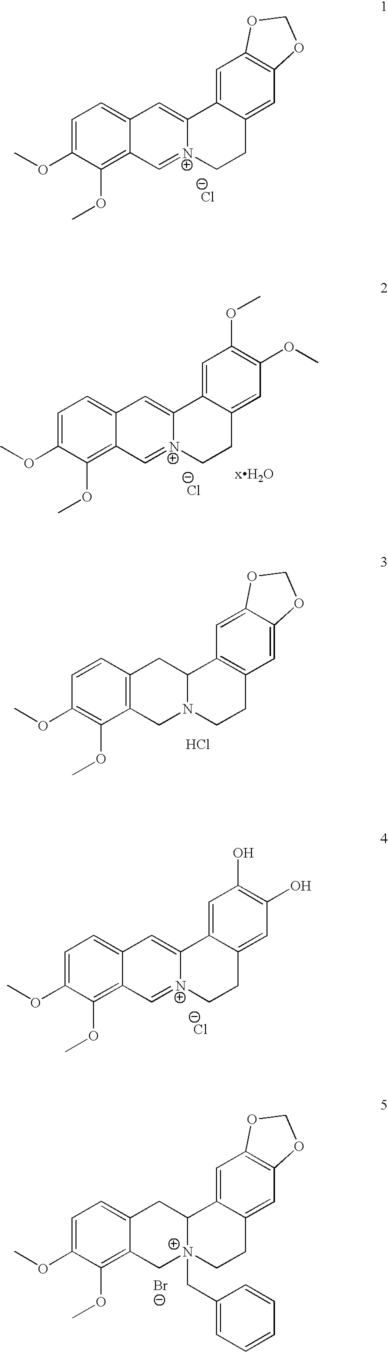

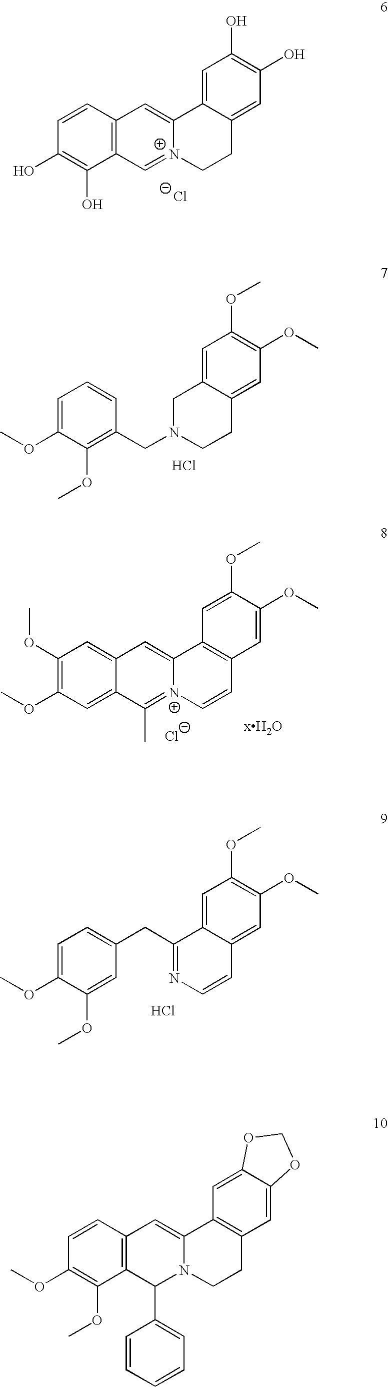

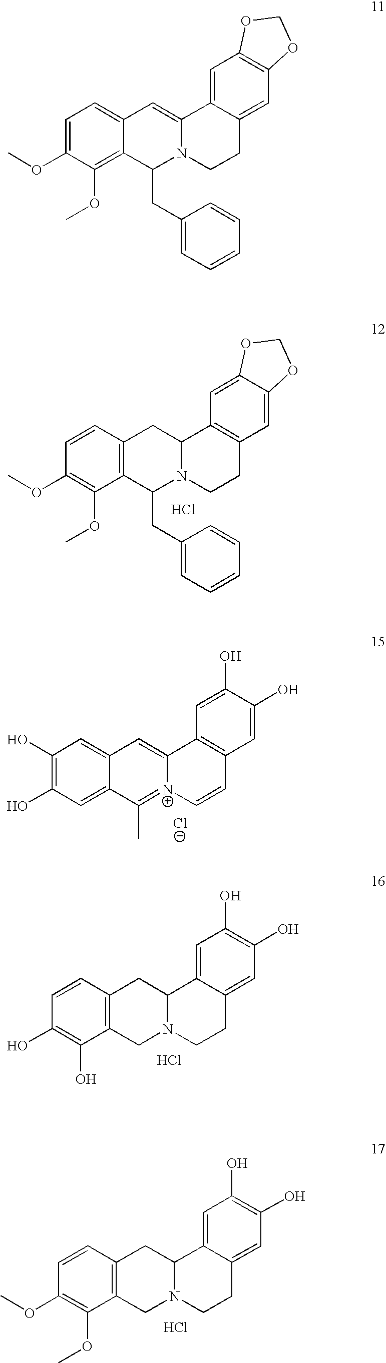

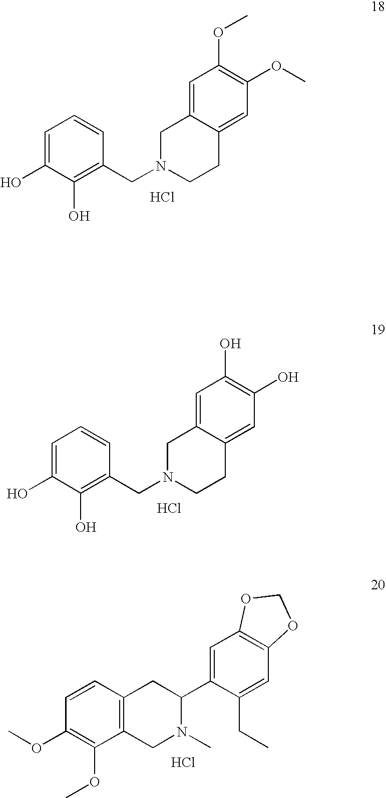

### 3-Arylisoquinoline Derivatives and Analogs

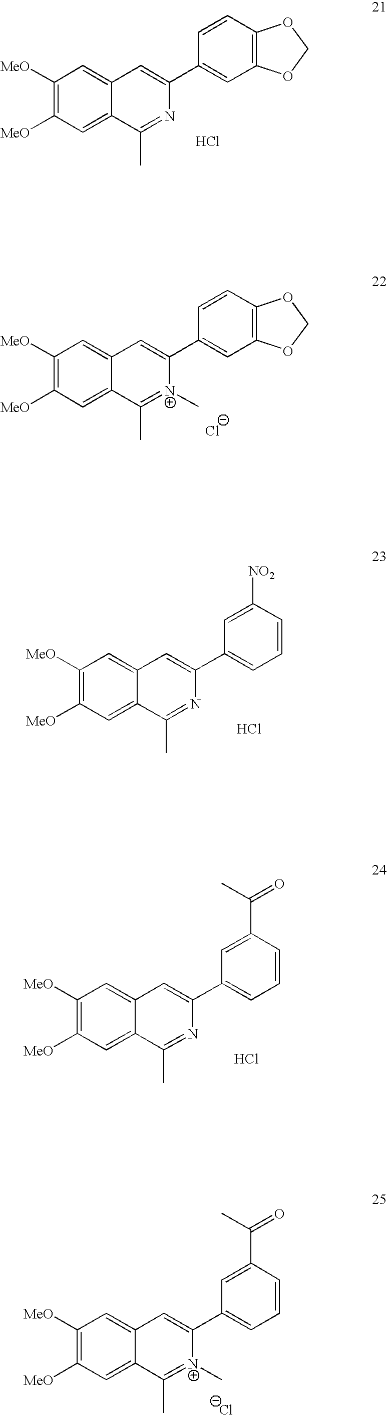

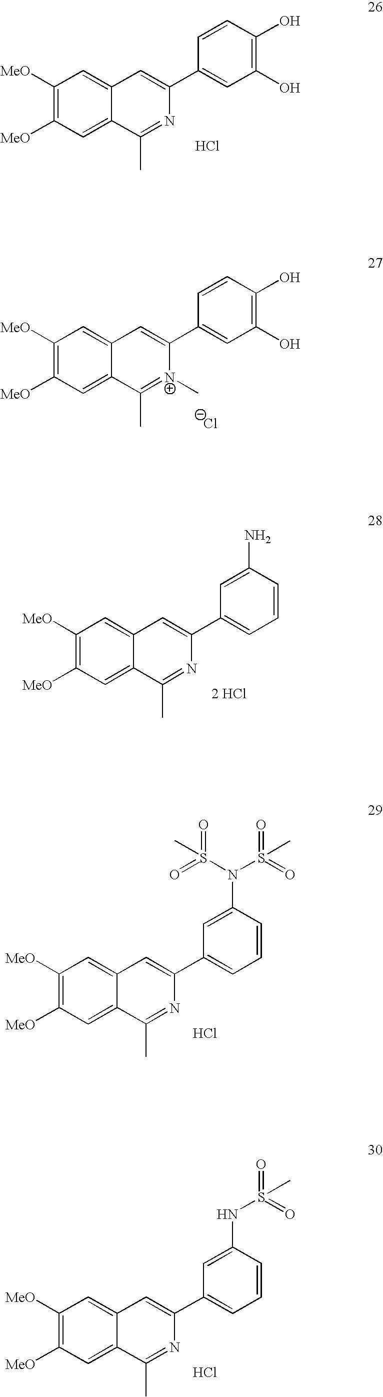

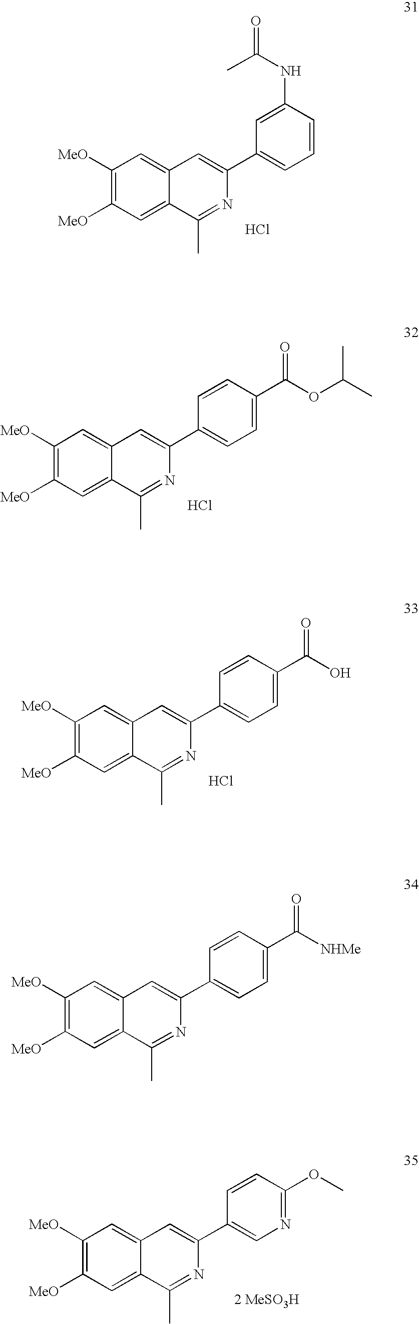

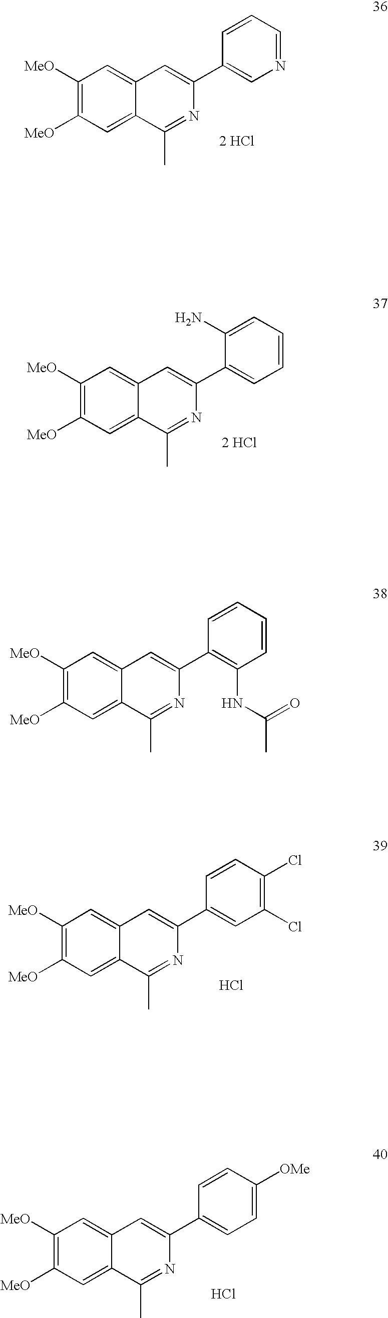

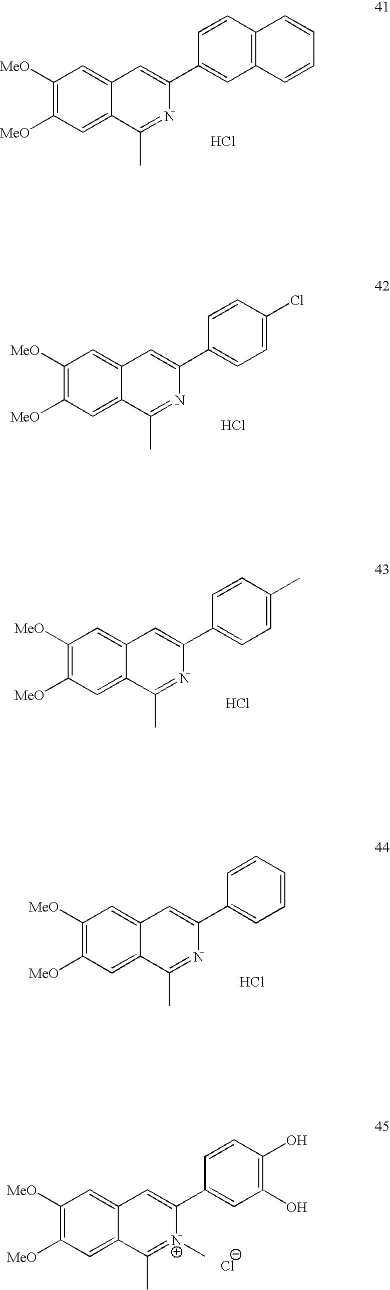

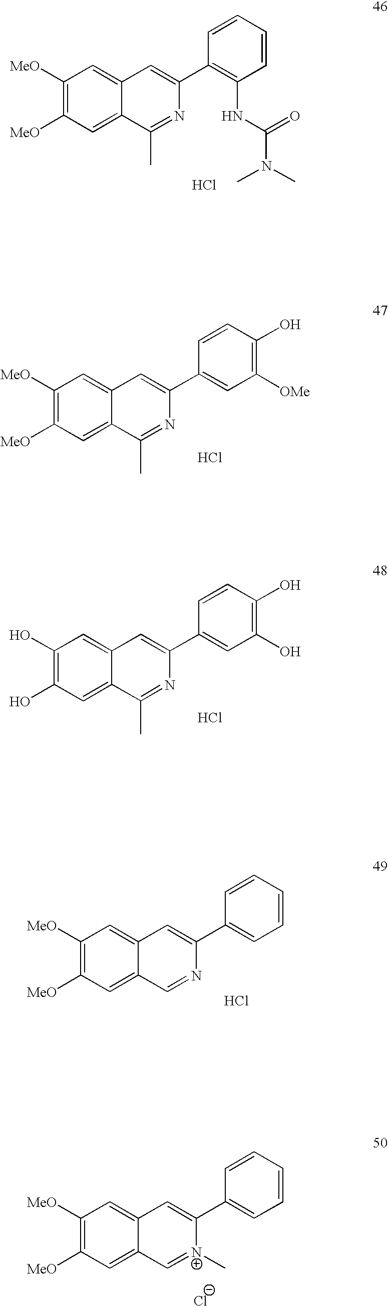

### Benzo[c]Phenanthridine Alkaloids

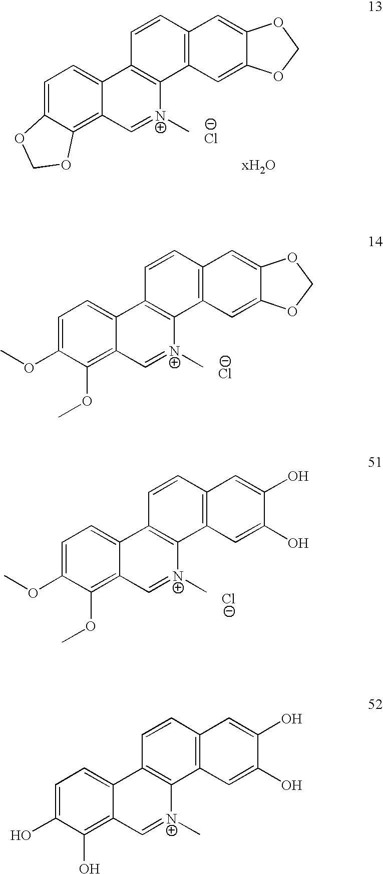

In a particular embodiment, the in vitro method of the present invention comprises contacting a member of the Rho GTPase family with a compound of formula (I) selected from the group consisting of demethyleneberberine 4, 2,3,9,10-tetrahydroxyberberine 6 and coralyne hydrochloride 8, 8-methyl-isoquino[3,2-a]isoquinolinylium-2,3,10,11-tetraol chloride 15, (±)-tetrahydroxytetrahydroberberine or (±)-5,8,13,13a-tetrahydro-6H-isoquino[3,2-a]isoquinolinylium-2,3,9,10-tetraol hydrochloride 16, 4-(6,7-dimethoxy-1-methylisoquinolin-3-yl)benzene-1,2-diol hydrochloride 26, isopropyl 4-(6,7-dimethoxy-1-methylisoquinolin-3-yl)benzoate hydrochloride 32, 3-(3,4-dichlorophenyl)-6,7-dimethoxy-1-methylisoquinolinylium chloride 39, 6,7-dimethoxy-1-methyl-3-(naphthalen-2-yl)isoquinolinylium chloride 41 and 3-(3,4-dihydroxyphenyl)-6,7-dihydroxy-1,2-dimethylisoquinolinium chloride 45, or with a compound of formula (II) selected from the group consisting of sanguinarine or 13-methyl-[1,3]benzodioxolo[5,6-c]-1,3-dioxolo[4,5-i]phenanthridinium chloride hydrate 13, chelerythrine or 1,2-dimethoxy-N-methyl[1,3]benzodioxolo[5,6-c]phenanthridinium chloride 14, 2,3-dihydroxy-7,8-dimethoxy-5-methylbenzo[c]phenanthridinium chloride 51 and 2,3,7,8-tetrahydroxy-5-methylbenzo[c]phenanthridinium chloride 52.

As indicated above, Rho family proteins constitute one of three major branches of the Ras superfamily. At least 14 mammalian Rho family proteins have been identified so far, including RhoA, RhoB, RhoC, RhoE/Rnd3, Rnd1/Rho6, Rnd2/Rho7, RhoG, Rac1, Rac1b, Rac2, Rac3, Cdc42, TC10, and TTF.

The present invention discloses experiments showing that compounds of formula (I) or (II) are effective inhibitors of members of the Rho GTPase family, in particular of the Rac GTPase subfamily, more particularly of Cdc42, Rac1 and/or Rac1b.

Accordingly, the invention relates more particularly to an in vitro method for modulating, preferably inhibiting, a Rho GTPase selected from the group consisting of RhoA, RhoB, RhoC, RhoE/Rnd3, Rnd1/Rho6, Rnd2/Rho7, RhoG, Rac1, Rac1b, Rac2, Rac3, Cdc42, TC10, and TTF. Particularly preferred GTPases are Cdc42, Rac1 and/or Rac1b wherein said GTPase is contacted with a compound of formula (I) or (II) as defined above, including a compound of any particular embodiment disclosed above.

According to another embodiment, the invention provides an in vitro method for modulating, preferably inhibiting, a Cdc42 or a Rac GTPase, preferably Rac1 and/or Rac1b.

In another particular embodiment, the in vitro method of the invention is implemented for modulating, preferably inhibiting, a Rac GTPase selected from the group consisting of Rac1, Rac 1b, Rac2, Rac3 and mixture thereof. In a further embodiment of the method of the invention, the Rac GTPase is selected from the group consisting of Rac1, Rac1b and mixture thereof.

In a further embodiment of the invention, the method of the invention comprises contacting a compound of formula (II), preferably sanguinarine 13, chelerythrine 14 or 2,3,7,8-tetrahydroxy-5-methylbenzo[c]phenanthridinium chloride 52, with Rac1b.

Another object of the invention provides a compound of formula (I) or (II) as defined above. In a particular embodiment, the invention relates to compound 8-methyl-isoquino[3,2-a]isoquinolinylium-2,3,10,11-tetraol chloride 15.

In particular, an object of the invention provides a compound of formula (V) as defined above, including the particular compounds described in embodiments (i)-(xvi) above. In a particular embodiment, the invention relates to compounds:
6,7-Dimethoxy-1-methyl-3-(3-nitrophenyl)isoquinolinium chloride 23,
1-(3-(6,7-Dimethoxy-1-methylisoquinolin-3-yl)phenyl)ethanone hydrochloride 24,
3-(3-Acetylphenyl)-6,7-dimethoxy-1,2-dimethylisoquinolinium chloride 25,
4-(6,7-Dimethoxy-1-methylisoquinolin-3-yl)benzene-1,2-diol hydrochloride 26,
3-(3,4-Dihydroxyphenyl)-6,7-dimethoxy-1,2-dimethylisoquinolinium chloride 27,
3-(6,7-Dimethoxy-1-methylisoquinolin-3-yl)aniline dihydrochloride 28,
N-(3-(6,7-Dimethoxy-1-methylisoquinolin-3-yl)phenyl)-N-(methylsulfonyl)methanesulfonamide hydrochloride 29,
N-(3-(6,7-Dimethoxy-1-methylisoquinolin-3-yl)phenyl)methanesulfonamide hydrochloride 30,
N-(3-(6,7-Dimethoxy-1-methylisoquinolin-3-yl)phenyl)acetamide hydrochloride 31,
Isopropyl 4-(6,7-dimethoxy-1-methylisoquinolin-3-yl)benzoate hydrochloride 32,
4-(6,7-Dimethoxy-1-methylisoquinolin-3-yl)benzoic acid hydrochloride 33,
4-(6,7-Dimethoxy-1-methylisoquinolin-3-yl)-N-methylbenzamide 34,
6,7-Dimethoxy-3-(6-methoxypyridin-3-yl)-1-methylisoquinoline dimethanesulfonate 35,
6,7-Dimethoxy-1-methyl-3-(pyridin-3-yl)isoquinoline dihydrochloride 36,
2-(6,7-Dimethoxy-1-methylisoquinolin-3-yl)aniline dihydrochloride 37,
N-(2-(6,7-Dimethoxy-1-methylisoquinolin-3-yl)phenyl)acetamide 38,
3-(3,4-Dichlorophenyl)-6,7-dimethoxy-1-methylisoquinolinylium chloride 39,
6,7-Dimethoxy-3-(4-methoxyphenyl)-1-methylisoquinolinylium chloride 40,
6,7-Dimethoxy-1-methyl-3-(naphthalen-2-yl)isoquinolinylium chloride 41,
3-(4-Chlorophenyl)-6,7-dimethoxy-1-methylisoquinolinylium chloride 42,
6,7-Dimethoxy-1-methyl-3-p-tolylisoquinolinylium chloride 43,
3-(2-(6,7-Dimethoxy-1-methylisoquinolin-3-yl)phenyl)-1,1-dimethylurea hydrochloride 46,
4-(6,7-Dimethoxy-1-methylisoquinolin-3-yl)-2-methoxyphenol hydrochloride 47,
4-(6,7-dimethoxy-1-methylisoquinolin-3-yl)benzene-1,2-diol hydrochloride 48.

In a preferred embodiment, the invention relates to the following compounds of formula (V):
4-(6,7-Dimethoxy-1-methylisoquinolin-3-yl)benzene-1,2-diol hydrochloride 26,
Isopropyl 4-(6,7-dimethoxy-1-methylisoquinolin-3-yl)benzoate hydrochloride 32,
3-(3,4-Dichlorophenyl)-6,7-dimethoxy-1-methylisoquinolinylium chloride 39,
6,7-Dimethoxy-1-methyl-3-(naphthalen-2-yl)isoquinolinylium chloride 41.

Another object of the invention relates to a compound of formula (II), in particular to compounds:
2,3-Dihydroxy-7,8-dimethoxy-5-methylbenzo[c]phenanthridinium chloride 51, and
2,3,7,8-Tetrahydroxy-5-methylbenzo[c]phenanthridinium chloride 52.

Another object of the invention relates to a compound of formula (I), in particular of formula (V), or a compound of formula (II), as a medicament. In particular, the invention relates to an 8-methyl-isoquino[3,2-a]isoquinolinylium-2,3,10,11-tetraol salt, and preferably an halide salt such as 8-methyl-isoquino[3,2-a]isoquinolinylium-2,3,10,11-tetraol chloride 15, as a medicament. The invention also relates to a compound of formula (I) selected from the group consisting of demethyleneberberine 4, 2,3,9,10-tetrahydroxyberberine 6 and coralyne hydrochloride 8, 8-methyl-isoquino[3,2-a]isoquinolinylium-2,3,10,11-tetraol chloride 15, (±)-tetrahydroxytetrahydroberberine or (±)-5,8,13,13a-tetrahydro-6H-isoquino[3,2-a]isoquinolinylium-2,3,9,10-tetraol hydrochloride 16, 4-(6,7-dimethoxy-1-methylisoquinolin-3-yl)benzene-1,2-diol hydrochloride 26, isopropyl 4-(6,7-dimethoxy-1-methylisoquinolin-3-yl)benzoate hydrochloride 32, 3-(3,4-Dichlorophenyl)-6,7-dimethoxy-1-methylisoquinolinylium chloride 39, 6,7-dimethoxy-1-methyl-3-(naphthalen-2-yl)isoquinolinylium chloride 41 and 3-(3,4-dihydroxyphenyl)-6,7-dihydroxy-1,2-dimethylisoquinolinium chloride 45, or with a compound of formula (II) selected from the group consisting of sanguinarine or 13-methyl-[1,3]benzodioxolo[5,6-c]-1,3-dioxolo[4,5-i]phenanthridinium chloride hydrate 13, chelerythrine or 1,2-dimethoxy-N-methyl[1,3]benzodioxolo[5,6-c]phenanthridinium chloride 14, 2,3-dihydroxy-7,8-dimethoxy-5-methylbenzo[c]phenanthridinium chloride 51 and 2,3,7,8-tetrahydroxy-5-methylbenzo[c]phenanthridinium chloride 52, as a medicament.

In a particularly preferred embodiment, the invention relates to a compound selected in the group consisting of 4-(6,7-dimethoxy-1-methylisoquinolin-3-yl)benzene-1,2-diol hydrochloride 26, isopropyl 4-(6,7-dimethoxy-1-methylisoquinolin-3-yl)benzoate hydrochloride 32, 3-(3,4-dichlorophenyl)-6,7-dimethoxy-1-methylisoquinolinylium chloride 39, 6,7-dimethoxy-1-methyl-3-(naphthalen-2-yl)isoquinolinylium chloride 41, 2,3-dihydroxy-7,8-dimethoxy-5-methylbenzo[c]phenanthridinium chloride 51, and 2,3,7,8-tetrahydroxy-5-methylbenzo[c]phenanthridinium chloride 52, as a medicament.

A further object of the invention relates to a pharmaceutical composition comprising at least one compound of formula (I), in particular a compound of formula (V), or formula (II), as defined above, and a pharmaceutically acceptable vehicle or support.

In a preferred embodiment of the invention, the pharmaceutical composition of the invention comprises compound 8-methyl-isoquino[3,2-a]isoquinolinylium-2,3,10,11-tetraol salt, and preferably an halide salt, such as 8-methyl-isoquino[3,2-a]isoquinolinylium-2,3,10,11-tetraol chloride 15 and a pharmaceutically acceptable vehicle or support.

In another preferred embodiment of the invention, the pharmaceutical composition of the invention comprises a pharmaceutically acceptable vehicle or support in mixture with at least one compound selected in the group consisting of 4-(6,7-dimethoxy-1-methylisoquinolin-3-yl)benzene-1,2-diol hydrochloride 26, isopropyl 4-(6,7-dimethoxy-1-methylisoquinolin-3-yl)benzoate hydrochloride 32, 3-(3,4-dichlorophenyl)-6,7-dimethoxy-1-methylisoquinolinylium chloride 39, 6,7-dimethoxy-1-methyl-3-(naphthalen-2-yl)isoquinolinylium chloride 41, 2,3-dihydroxy-7,8-dimethoxy-5-methylbenzo[c]phenanthridinium chloride 51, and 2,3,7,8-tetrahydroxy-5-methylbenzo[c]phenanthridinium chloride 52.

The compounds may be formulated in various forms, including solid and liquid forms, such as tablets, gel, syrup, powder, aerosol, etc.

The compositions of this invention may contain physiologically acceptable diluents, fillers, lubricants, excipients, solvents, binders, stabilizers, and the like. Diluents that may be used in the compositions include but are not limited to dicalcium phosphate, calcium sulphate, lactose, cellulose, kaolin, mannitol, sodium chloride, dry starch, powdered sugar and for prolonged release tablet-hydroxy propyl methyl cellulose (HPMC). The binders that may be used in the compositions include but are not limited to starch, gelatin and fillers such as sucrose, glucose, dextrose and lactose.

Natural and synthetic gums that may be used in the compositions include but are not limited to sodium alginate, ghatti gum, carboxymethyl cellulose, methyl cellulose, polyvinyl pyrrolidone and veegum. Excipients that may be used in the compositions include but are not limited to microcrystalline cellulose, calcium sulfate, dicalcium phosphate, starch, magnesium stearate, lactose, and sucrose. Stabilizers that may be used include but are not limited to polysaccharides such as acacia, agar, alginic acid, guar gum and tragacanth, amphotsics such as gelatin and synthetic and semi-synthetic polymers such as carbomer resins, cellulose ethers and carboxymethyl chitin.

Solvents that may be used include but are not limited to Ringers solution, water, distilled water, dimethyl sulfoxide to 50% in water, propylene glycol (neat or in water), phosphate buffered saline, balanced salt solution, glycol and other conventional fluids.

The dosages and dosage regimen in which the compounds of formula (I) or (II) are administered will vary according to the dosage form, mode of administration, the condition being treated and particulars of the patient being treated. Accordingly, optimal therapeutic concentrations will be best determined at the time and place through routine experimentation.

The compounds of formula (I) or (II) can also be used enterally. Orally, the compounds according to the invention are suitable administered at the rate of 100 μg to 100 mg per day per kg of body weight. The required dose can be administered in one or more portions. For oral administration, suitable forms are, for example, tablets, gel, aerosols, pills, dragees, syrups, suspensions, emulsions, solutions, powders and granules; a preferred method of administration consists in using a suitable form containing from 1 mg to about 500 mg of active substance.

The compounds according to the invention can also be administered parenterally in the form of solutions or suspensions for intravenous or intramuscular perfusions or injections. In that case, the compounds according to the invention are generally administered at the rate of about 10 μg to 10 mg per day per kg of body weight; a preferred method of administration consists of using solutions or suspensions containing approximately from 0.01 mg to 1 mg of active substance per ml.

Another object of the invention relates to a compound of formula (I) or (II), including a compound of any particular embodiment disclosed above, for the manufacture of a pharmaceutical composition for treating a pathology involving a member of the Rho GTPase family.

In a particular embodiment of the invention, the pathology involving a member of the Rho GTPase family is selected from the group consisting of platelet hyperreactivity, hypertension, atherosclerosis, restenosis, cerebral ischemia, cerebral vasospasm, neurodegenerative pathologies, spinal cord injury, cancer of the breast, colon, prostate, ovaries, brain or lung, thrombotic disorders, asthma, glaucoma, osteoporosis and erectile dysfunction.

In a particular embodiment, the disease associated with Rho GTPase activity, preferably Rac GTPases activity, is selected from cancer and neurodegenerative pathologies, in particular Alzheimer Disease.

Preferred compounds for use according to the invention include any sub-group as defined above and any specific compounds as identified above.

Another object of the invention is a compound of formula (I) or (II) as defined above, including a compound of any particular embodiment disclosed above, for the treatment of a pathology involving a member of the Rho GTPase family as defined above.

In a particular embodiment, the invention relates to a compound of formula (II) for the treatment of cancer, in particular of cancer of the breast, colon, prostate, ovaries, brain or lung. Preferably, the compound of formula (II) is selected in the group consisting of sanguinarine or 13-methyl-[1,3]benzodioxolo[5,6-c]-1,3-dioxolo[4,5-i]phenanthridinium chloride hydrate 13, chelerythrine or 1,2-dimethoxy-N-methyl[1,3]benzodioxolo[5,6-c]phenanthridinium chloride 14, 2,3-dihydroxy-7,8-dimethoxy-5-methylbenzo[c]phenanthridinium chloride 51 and 2,3,7,8-tetrahydroxy-5-methylbenzo[c]phenanthridinium chloride 52. Particularly preferred is a compound selected in the group consisting of 2,3-dihydroxy-7,8-dimethoxy-5-methylbenzo[c]phenanthridinium chloride 51 and 2,3,7,8-tetrahydroxy-5-methylbenzo[c]phenanthridinium chloride 52 for the treatment of cancer, in particular of cancer of the breast, colon, prostate, ovaries, brain or lung.

A further object of the invention is a method for the treatment of a pathology involving a member of the Rho GTPase family, comprising administering to a patient in need of such treatment an effective amount of at least one compound of general formula (I) or (II) as described above, including a compound of any particular embodiment disclosed above.

“Treatment” or “treating” includes both therapeutic and prophylactic treatments. Accordingly, the compounds may be used at very early stages of a disease, or before early onset, or after significant progression, including metastasis. The term “treatment” or “treating” designates in particular a reduction of the burden in a patient, such as a reduction in cell proliferation rate, a destruction of diseased proliferative cells, a reduction of tumor mass or tumor size, a delaying of tumor progression, as well as a complete tumor suppression.

The compounds may be administered according to various routes, typically by injection, such as local or systemic injection(s). Intratumoral injections are preferred for treating existing cancers. However, other administration routes may be used as well, such as intramuscular, intravenous, intradermic, subcutaneous, etc. Furthermore, repeated injections may be performed, if needed, although it is believed that limited injections will be needed in view of the efficacy of the compounds.

Further aspects and advantages of this invention will be disclosed in the following examples, which should be regarded as illustrative and not limiting the scope of this application.

### BRIEF DESCRIPTION OF THE DRAWINGS

FIG. 1: IC₅₀s for protoberberine derivatives 4 and 6

FIG. 2: IC₅₀s for protoberberine derivatives 15 and 16

FIG. 3: IC₅₀s for benzo[c]phenanthridine alkaloids 13 and 14

FIG. 4: IC₅₀s for benzo[c]phenanthridine alkaloids 51 and 52

FIG. 5: IC₅₀s for 3-aryl-isoquinolines 26 and 41

FIG. 6: Dose-response study for control compound NSC 23766

### EXAMPLES

Berberine chloride 1, palmatine chloride hydrate 2, papaverine or 1-(3,4-dimethoxybenzyl)-6,7-dimethoxyisoquinoline hydrochloride 9, 9,10-dimethoxy-8-phenyl-5,8-dihydro-2H-6H-[1,3]dioxolo[4,5-g]isoquino[3,2-a]isoquinoline 10, 8-benzyl-9,10-dimethoxy-5,8-dihydro-2H-6H-[1,3]dioxolo[4,5-g]isoquino[3,2-a]isoquinoline 11, sanguinarine chloride hydrate 13 and chelerythrine chloride 14 were obtained from Sigma-Aldrich (St. Louis, Mo., USA). Coralyne chloride hydrate 8 was obtained from Acros Organics (New Jersey, USA).

Compounds 3 to 6 and 16-17 were prepared from berberine chloride 1 by the following synthetic routes summarized below:

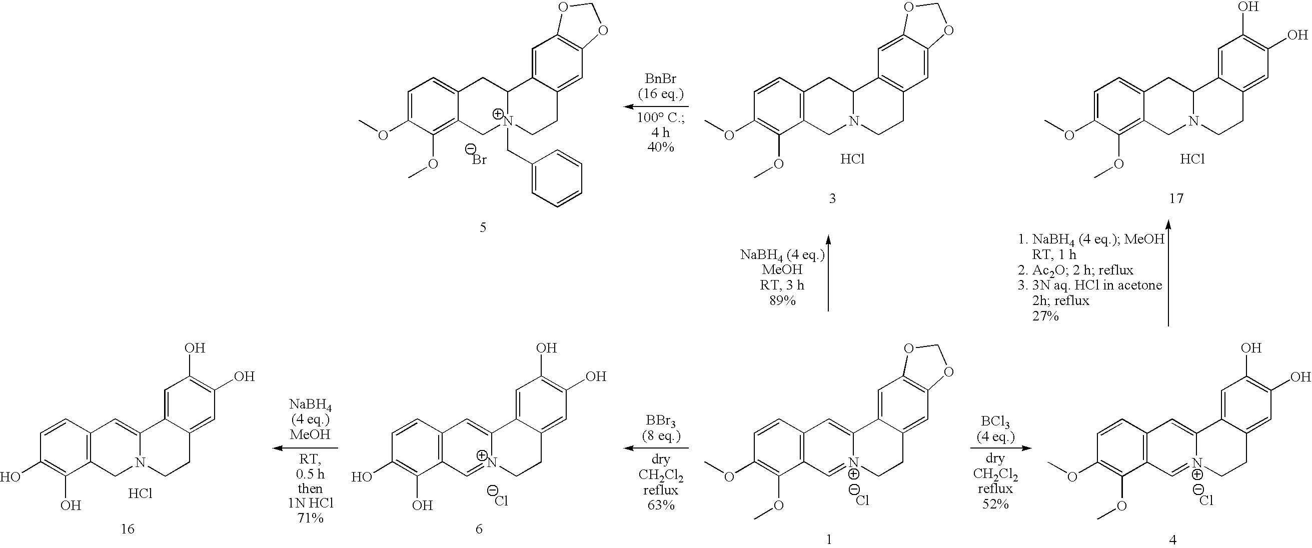

(±)-Canadine hydrochloride 3 was prepared by sodium borohydride reduction of berberine chloride 1 in solution in MeOH (adapted from Ito K., Yagugaku Zasshi, 1960, 80, 705) and followed by treatment with an ethanolic HCl solution. (±)-N-benzyl canadinium bromide 5 was obtained by reaction of (±)-canadine with an excess of benzyl bromide at reflux for 4 h (adapted from Kametani T., Taguchi E., Yamaki K., Kozuka A., and Terui T., Chem. Pharm. Bull., 1973, 21(5), 1124-1126).

Demethylene berberine chloride 4 was prepared by demethylation of berberine chloride 1 using 4 equivalents of boron trichloride in dichloromethane at reflux (adapted from Hanaoka, M., Nagami, K., Hirai, Y., Sakurai, S.-H., and Yasuda, S., Chem. Pharm. Bull., 1985, 33(6), 2273-2280.). Berberine chloride 1 treated by an excess of boron tribromide in dry dichloromethane at reflux afforded 2,3,9,10-tetrahydroxyberberine chloride 6 (adapted from Colombo M. L., Bugatti, C., Mossa A., Pescalli N., Piazzoni L., Pezzoni G., Menta E., Spinelli S., Johnson F., Gupta R. C., and Dasaradhi L., Il Farmaco, 2001, 56, 403-409.).

(±)-Tetrahydroxytetrahydroberberine hydrochloride 16 was prepared by sodium borohydride reduction of 2,3,9,10-tetrahydroxyberberine chloride 6 in solution in MeOH followed by treatment with 1 N aqueous HCl solution. Compound 16 has also been prepared in the literature by an alternative method (Colombo M. L., Bugatti, C., Mossa A., Pescalli N., Piazzoni L., Pezzoni G., Menta E., Spinelli S., Johnson F., Gupta R. C., and Dasaradhi L., Il Farmaco, 2001, 56, 403-409.).

(±)-9,10-Dimethoxy-5,8,13,13a-tetrahydro-6H-isoquino[3,2-a] isoquinoline-2,3-diol hydrochloride 17 was prepared by sodium borohydride reduction of demethylene berberine chloride 4 in solution in MeOH. The obtained crude product was acetylated for 2 hours at reflux using acetic anhydride then subsequently hydrolyzed with a 3N aqueous HCl solution in acetone 2 hours at reflux to finally afford compound 17 as a hydrochloride salt.

Compound 7 was prepared by reductive amination using sodium triacetoxyborohydride, 6,7-dimethoxy-1,2,3,4-tetrahydro isoquinoline hydrochloride and 2,3-dimethoxybenzaldehyde. Compound 7 has also been prepared by an alternative method in the literature (Kiparissides, Zinovia et al., Can. J. Chem., 1958, 58, 2770-2779).

2-(2,3-Dihydroxybenzyl)-6,7-dihydroxy-1,2,3,4-tetrahydroisoquinolinium chloride 19 was obtained from compound 7 by treatment for 15 hours with a 1M BBr₃ solution in dichloromethane followed by methanolic HCl treatment.

Compound 18 was prepared by reductive amination using sodium triacetoxyborohydride, 6,7-dimethoxy-1,2,3,4-tetrahydro isoquinoline hydrochloride and 2,3-dihydroxybenzaldehyde.

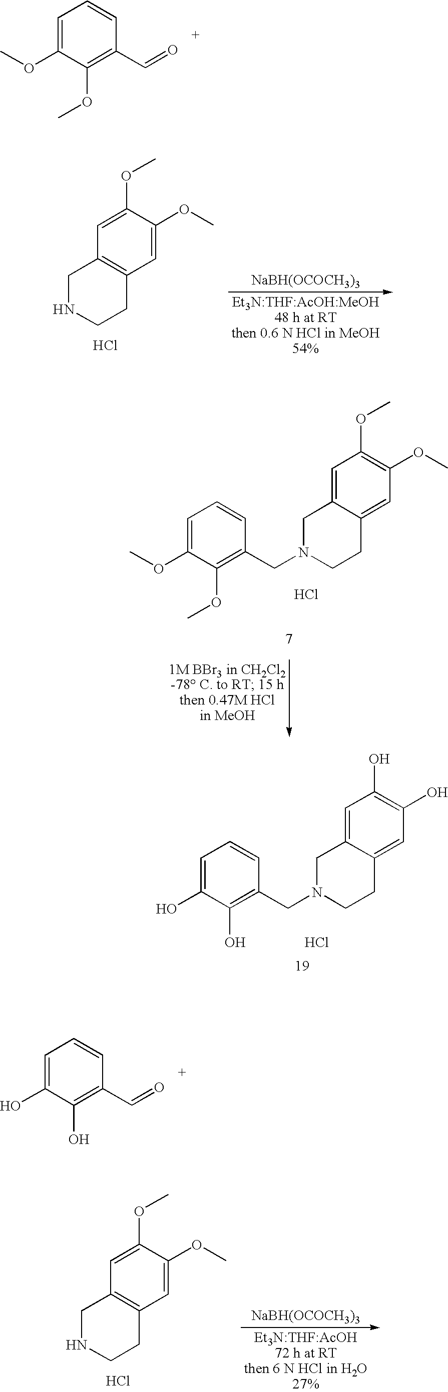

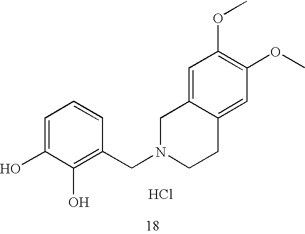

Compound 12 was prepared from (±)-8-benzyl-9,10-dimethoxy-5,8,13,13a-tetrahydro-6H-[1,3]dioxolo[4,5-g]isoquino[3,2-a]isoquinoline obtained from Sigma-Aldrich (St. Louis, Mo., USA). Its hydrochloride salt was purified by preparative HPLC and regenerated as an hydrochloride salt with a methanolic HCl solution.

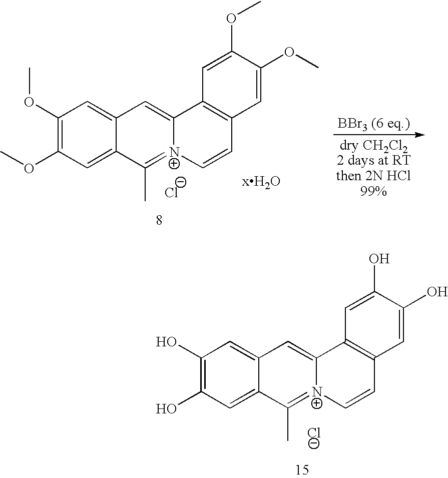

Coralyne chloride hydrate 8 treated by an excess of boron tribromide in dry dichloromethane for 2 days at RT, followed by 2 N aqueous HCl treatment, afforded to 8-methyl-isoquino[3,2-a]isoquinolinylium-2,3,10,11-tetraol chloride 15. An alternative synthetic preparation of compound 15 has been described in Japanese Patent JP 51034200.

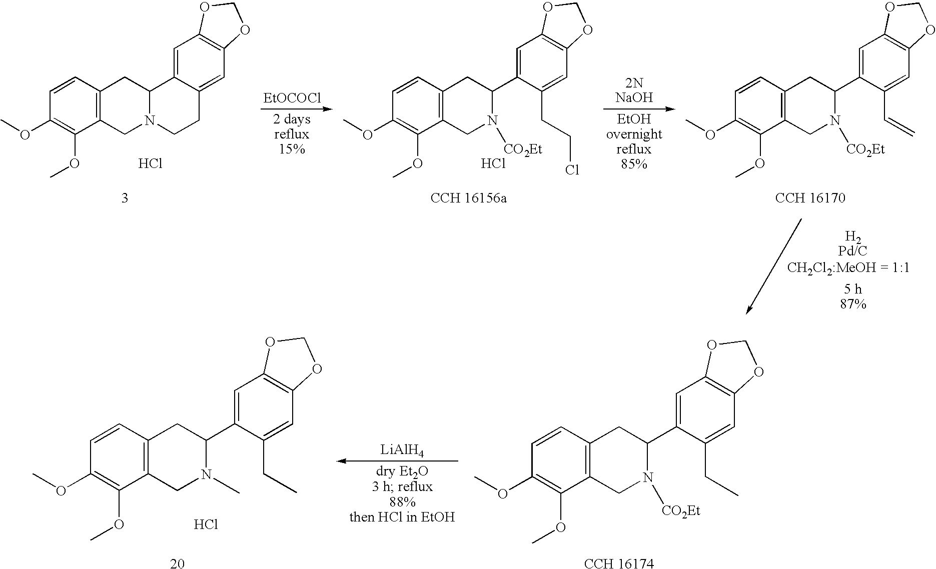

Compound 20 was prepared in four steps from (±)-tetrahydroberberine hydrochloride 3. Reaction of 3 with ethyl chloroformate for 2 days under reflux afforded the chloro intermediate CCH 16156a. Preparation of CCH 16156a was described by M. Hanaoka et al., Chem. Pharm. Bull., 1983, 31, 2685-2690. CCH 16156a was deshydrochlorinated by an overnight treatment with a 2N aqueous NaOH solution at reflux in ethanol to give the vinylic derivative CCH 16170 that was subsequently reduced by hydrogenation over 10% Pd/C for 5 hours in a 1:1 mixture of dichloromethane and methanol to obtain after work-up the intermediate CCH 16174. Final reaction of CCH 16174 with lithium borohydride for 3 hours in dry diethyl ether followed by hydrochloride treatment in ethanol afforded (±)-3-(6-ethylbenzo[d][1,3]dioxol-5-yl)-7,8-dimethoxy-2-methyl-1,2,3,4-tetrahydroisoquinolinylium chloride 20.

The synthetic methods employed for the preparation of the 3-aryl-isoquinolines are outlined in the Schemes below.

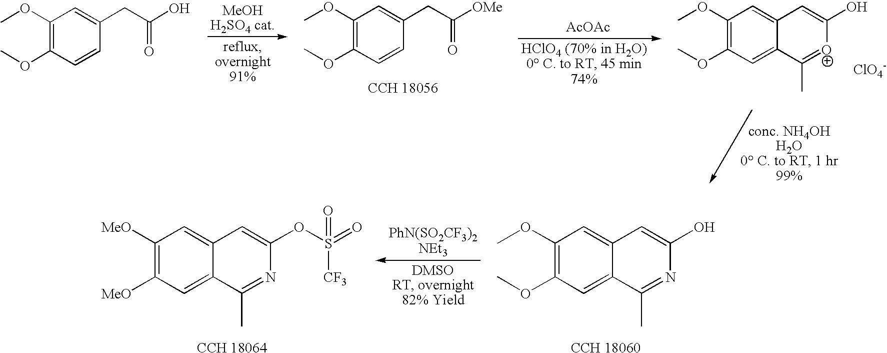

3,4-Dimethoxyphenylacetic acid was esterified in its corresponding methyl ester CCH 18056. This methyl ester reacted with acetic anhydride in presence of perchloric acid to obtain 3-hydroxy-6,7-dimethoxy-1-methylisochromenylium perchlorate that was immediately treated with a concentrated ammonium hydroxide solution to provide 6,7-dimethoxy-1-methylisoquinolin-3-ol CCH 18060. Preparation of CCH 18060 was described by R. M. Kanojia et al., J. Med. Chem., 1988, 31, 1363-1368. Compound CCH 18060 was finally treated with N-phenyl-bis(trifluoromethanesulfonimide) in presence of triethylamine at RT to afford the triflate intermediate CCH 18064.

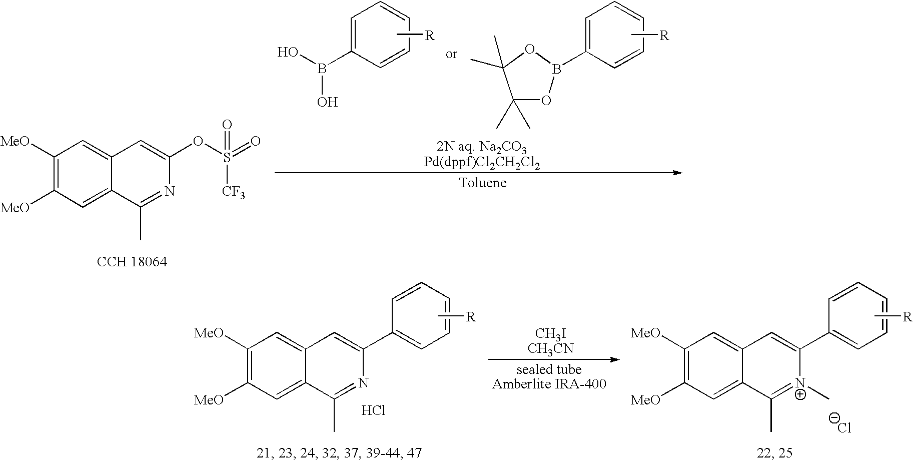

| Reagent | Condition | Compounds | Yield | Condition | Compounds | Yield |
|---|---|---|---|---|---|---|
| 3,4-(methylenedioxy)phenylboronic | overnight at 80° C. | 21 R = 3,4-methylenedioxy | 62% | 95° C. for 24 h | 22 R = 3,4- | 62% |
| acid | | | | | methylenedioxy | |
| 3-nitrophenylboronic acid | overnight at 80° C. | 23 R = 3-nitro | 60% | | | |
| 3-acetylphenylboronic acid | 4 h at 80° C. | 24 R = 3-acetyl | 41% | 3 days at 90° C. | 25 R = 3-acetyl | 64% |
| 4-isopropoxycarbonylphenylboronic | 4 h at 80° C. | 32 R = 4-isopropoxycarbonyl | 49% | | | |
| acid | | | | | | |
| 2-aminophenylboronic acid pinacol | overnight at 80° C. | 37 R = 2-amino | 52% | | | |
| ester | | | | | | |
| 3,4-dichlorophenylboronic acid | 1 h at 85° C. | 39 R = 3,4-dichloro | 42% | | | |
| 4-methoxyphenylboronic acid | 1 h at 85° C. | 40 R = 4-methoxy | 39% | | | |
| 2-naphthaleneboronic acid | 1 h at 85° C. | 41 R = 3,4-CH═CH—CH═CH— | 47% | | | |
| 4-chlorophenylboronic acid | 1 h at 85° C. | 42 R = 4-chloro | 55% | | | |
| 4-tolylboronic acid | 1 h at 85° C. | 43 R = 4-methyl | 52% | | | |
| phenylboronic acid | 1 h at 85° C. | 44 R = H | 94% | | | |
| 2-methoxy-4-(4,4,5,5-tetramethyl- | 1 h at 85° C. | 47 R = 4-OH, 3-OMe | 16% | | | |
| 1,3,2-dioxaborolan-2-yl)phenol | | | | | | |

Suzuki coupling between triflate CCH 18064 and substituted phenylboronic acids using [1,1′-bis(diphenylphosphino)ferrocene]dichloropalladium(II) complex as catalyst in toluene at 80-85° C. for 1 hour to overnight and in presence of 2N aqueous Na₂CO₃ afforded after work-up and treatment with HCl to isoquinolines 21, 23, 24, 32, 37, 39-44, and 47 in 16 to 94% yields.

Compounds 21 and 24 were converted to their corresponding 2-methylisoquinolinium 22 and 25, respectively, by treatment with methyl iodide.

Suzuki coupling between substituted triflate CCH 18064 and 2-methoxy-substituted or not pyridineboronic acids in conditions similar to above provided after work-up and treatment with methanesulfonic acid or HCl, respectively, to isoquinolines 35 and 36.

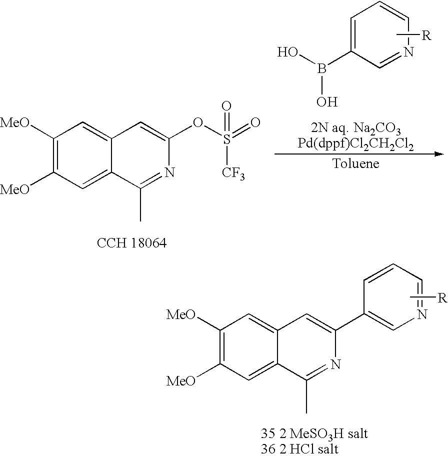

| | | | |
|---|---|---|---|
| 2-methoxy-5-pyridine- | 2.5 h at 80° C. | 35 R = 2-methoxy | 47% yield |
| boronic acid | | | |
| 3-pyridinylboronic | 4 h at 90° C. | 36 R = H | 9% yield |
| acid | | | |

Compounds 21 and 22 were already described: PCT/US97/01676 described the preparation of compounds 21 (as a free base) and 22 (as a methosulfate instead chloride) by an alternative method: a Friedel-Craft acylation of 1,2-(methylenedioxy)benzene with 3,4-dimethoxyphenyl-acetyl chloride provided a ketone intermediate that was cyclized by reaction with acetonitrile in P₂O₅ to afford compound 21 (as a free base). Compound 22 was obtained from 21 by treatment with dimethylsulfate.

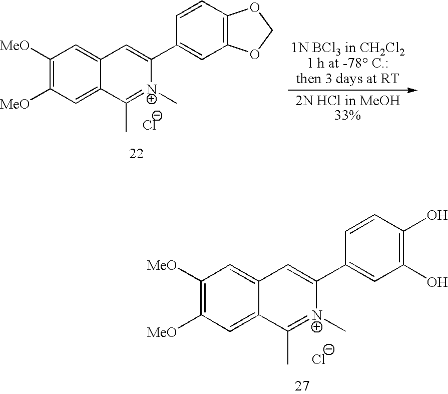

Compounds 26 and 27 were obtained from compounds 22 and CCH 18068 (21 free base), respectively, by treatment with a 1N boron trichloride solution.

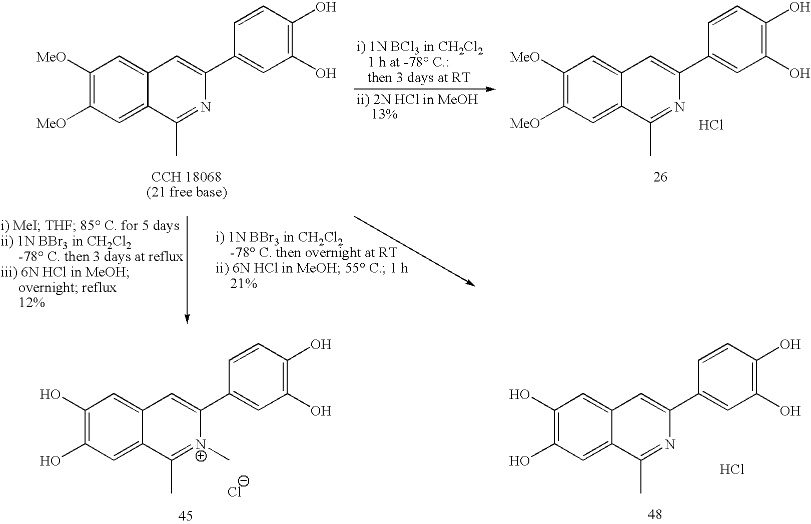

Compound 48 was obtained from compound CCH 18068 (21 free base) by treatment with a 1N boron trichloride solution followed by HCl treatment in MeOH.

Compound 45 was prepared from compound CCH 18068 (21 free base). Treatment of CCH 18068 by iodomethane in THF gave an N-methyl intermediate that was immediately O-demethylated by reaction with a 1N boron tribromide solution in dichloromethane to gave, after HCl treatment, the desired compound 45 in 12% overall yield.

Compounds 45 and 48 have been already described: PCT/US97/01676 described compound 48 (as a free base, obtained by treatment of 21 free base using borontribromide in chloroform) and 45 (methosulfate instead chloride) by treatment of compound 48 with dimethylsulfate.

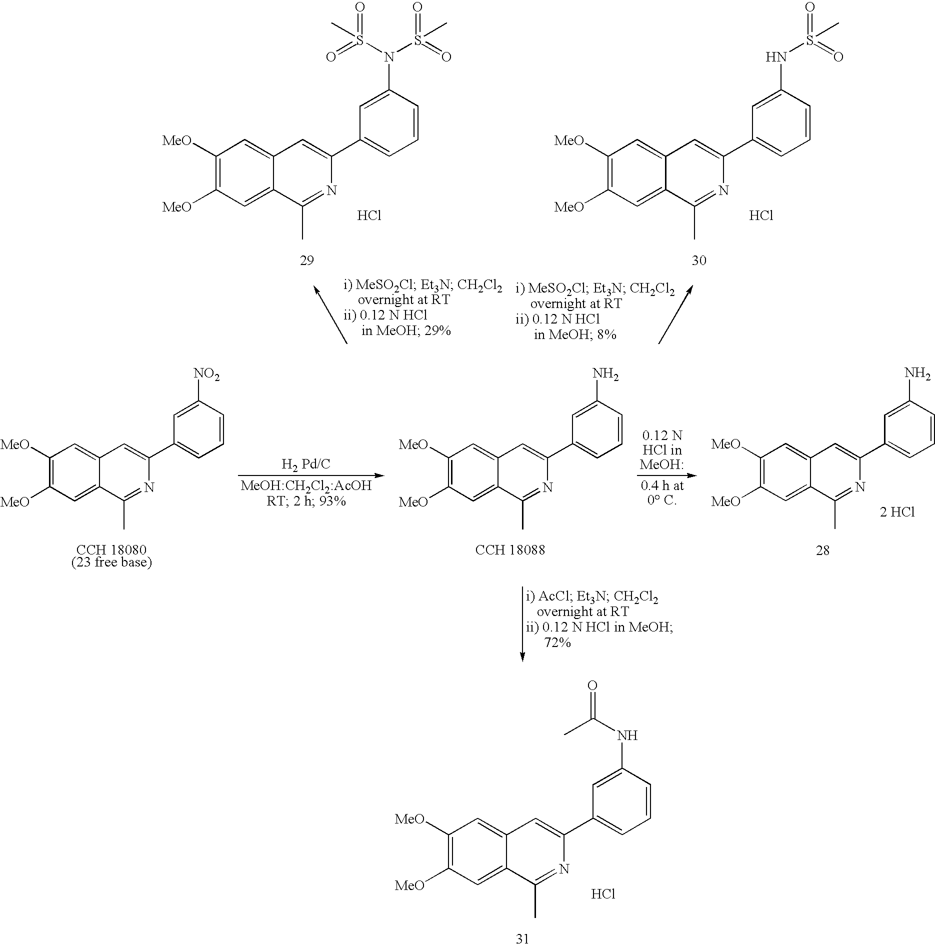

Hydrogenation of the nitro derivative CCH 18080 (23 free base) over 10% Pd/C for 2 hours in a mixture of dichloromethane, acetic acid and methanol gave aniline CCH 18088 in 93% yield. Aniline CCH 18088 was treated with a HCl solution in methanol to afford its corresponding bis HCl salt, compound 28.

Mono and bis methylsulfonamides 30 and 29 were prepared from aniline CCH 18088 by an overnight reaction at room temperature with methanesulfonyl chloride (respectively 1 or 2 equivalent) in dichloromethane in presence of triethylamine, followed by a final HCl treatment.

Aniline CCH 18080 was reacted overnight at room temperature with acetyl chloride in dichloromethane in presence of triethylamine to give after HCl treatment its N-acetyl derivative compound 31.

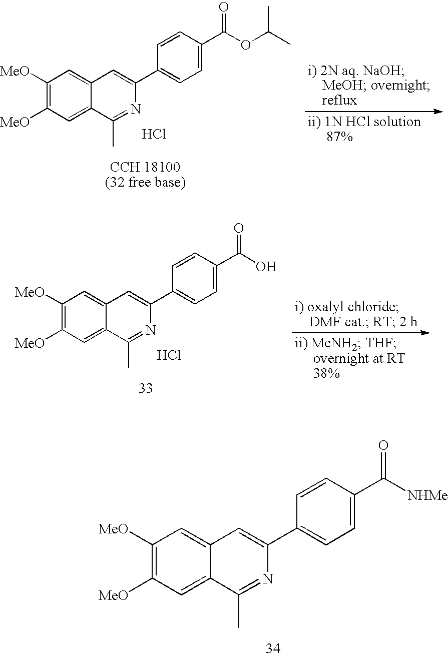

Carboxylic acid 33 was prepared from ester CCH 18100 (32 free base) by saponification, overnight at reflux, using a 2N solution of sodium hydroxyde in methanol, followed by work-up and final HCl treatment. Treatment for 2 hours at room temperature of acid 33 with oxalyl chloride in presence of catalytic amount of dimethylformamide afforded its corresponding acid chloride that was immediately treated overnight at room temperature with a solution of methylamine in water, in presence of THF, to give methylamine 34 in 38% yield.

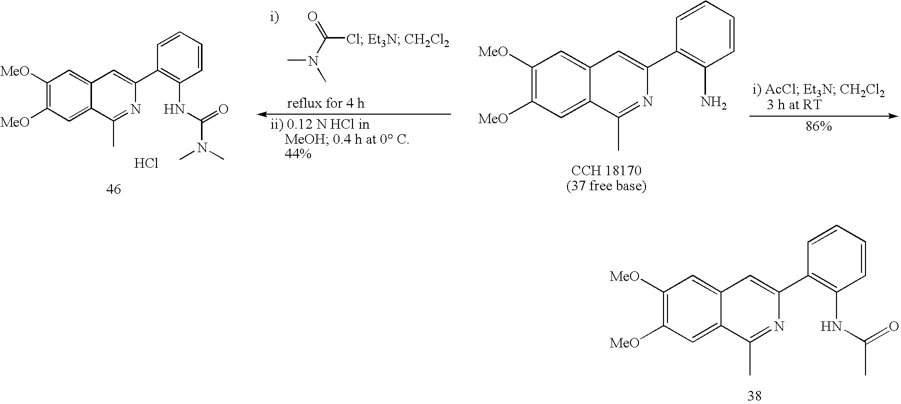

N-acetyl derivative 38 was obtained in 86% yield from aniline CCH 18170 (37 free base) by treatment with acetyl chloride in dichloromethane for 3 hours at room temperature, in presence of triethylamine.

Aniline CCH 18170 was treated with dimethylcarbamoyl chloride in dichloromethane in presence of triethylamine followed by a final HCl treatment to obtain 1,1-dimethylurea 46 in 44% yield.

Compound 49 was prepared in three synthetic steps. Phenylisocyanide in solution in THF at −78° C. was treated with a solution of 1.6M n-butyllithium in hexanes and quenched with a 3,4-dimethoxybenzaldehyde solution in THF to obtain (±)-trans-5-(3,4-dimethoxyphenyl)-4-phenyl-4,5-dihydrooxazole EBE 10166. Dihydrooxazole EBE 10166 was treated with phosphorus chloride oxide in acetonitrile to obtain 3-phenylisoquinoline EBE 10168. Finally 6,7-dimethoxy-3-phenylisoquinolinium chloride 49 was obtained by HCl treatment of 3-phenylisoquinoline EBE 10168.

Finally compound 49 was converted to its corresponding 2-methylisoquinolinium 50, by treatment with methyl iodide.

Compounds 49 and 50 have already been described and prepared by an alternative method in the literature (D. N. Harcourt and R. D. Waigh, J. Chem. Soc. C, 1971, 967-969).

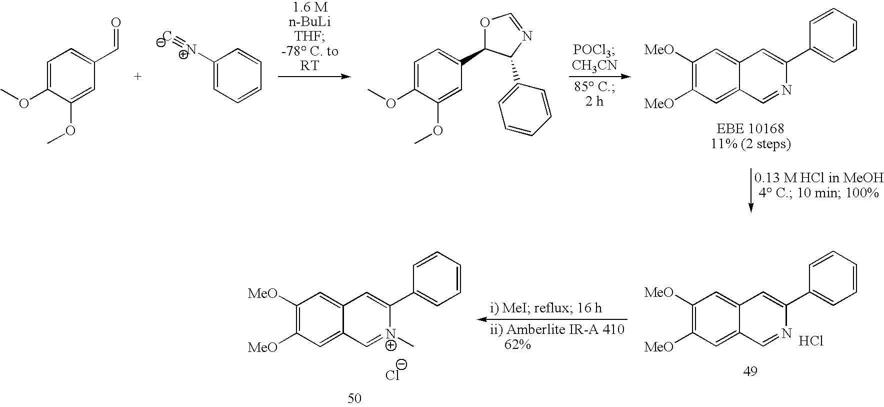

Compounds 51 and 52 were prepared as follows:

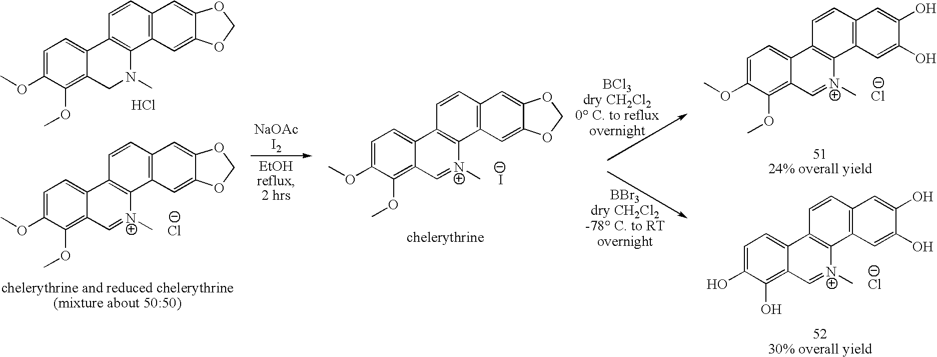

A mixture of chelerythrine with its reduced form (about a 50:50 mixture) in solution in ethanol was treated at reflux for 2 hours with iodine in presence of sodium acetate to give chelerythrine that was reacted with boron trichloride or boron tribromide, respectively, to afford compounds 51 and 52.

Herein below are presented the origin, synthesis and physico-chemical properties of compounds 1 to 52 according to formula (I) or (II).

### Compounds 1-2, 8-11, 13-14:

Berberine chloride 1, palmatine chloride hydrate 2, papaverine or 1-(3,4-dimethoxybenzyl)-6,7-dimethoxyisoquinoline hydrochloride 9, 9,10-dimethoxy-8-phenyl-5,8-dihydro-2H-6H-[1,3]dioxolo[4,5-g]isoquino[3,2-a]isoquinoline 10, 8-benzyl-9,10-dimethoxy-5,8-dihydro-2H-6H-[1,3]dioxolo[4,5-g]isoquino[3,2-a]isoquinoline 11, sanguinarine chloride hydrate 13 and chelerytrine chloride 14 were obtained from Sigma-Aldrich (St. Louis, Mo., USA). Coralyne chloride hydrate 8 was obtained from Acros Organics (New Jersey, USA).

### Protoberberine Class of Isoquinoline Alkaloids

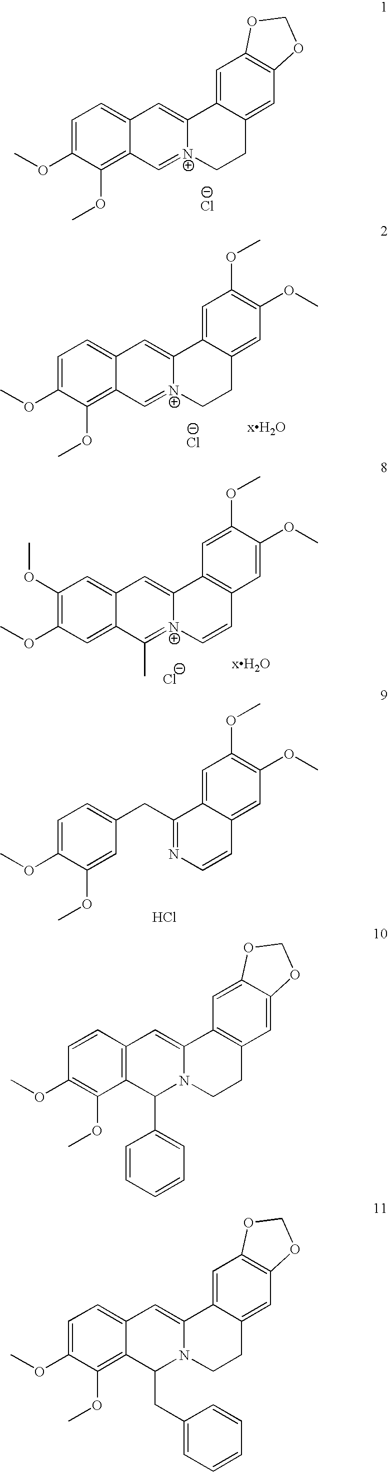

### Benzo[c]phenanthridine Alkaloids

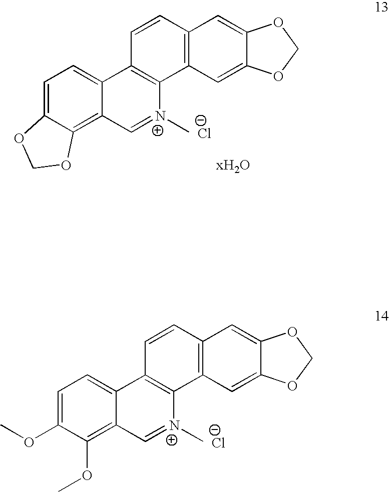

### Preparation of Compounds 3-7, 12 and 15-16

### (±)-Canadine or (±)-Tetrahydroberberine Hydrochloride 3

To a solution of berberine chloride (429 mg, 1.15 mmol) in MeOH (35 mL) was slowly added NaBH₄ (174 mg, 4.60 mmol) in a 100 mL round-bottomed flask equipped with a magnetic stirrer. The reaction mixture was stirred at RT for 3 h, after which MeOH was removed at 40° C. under vacuum. Water (10 mL) was added and the product was extracted with CH₂Cl₂ (30 mL), then with CH₂Cl₂:MeOH=5:1 (30 mL). The organic phases were combined, washed with brine (10 mL), dried (Na₂SO₄) and concentrated at 40° C. under vacuum, giving 348 mg of a yellow solid (89% yield). The solid was dissolved in MeOH (10 mL) in a 50 mL round-bottomed flask equipped with a magnetic stirrer and the solution was cooled to 0° C. in an ice bath before adding 3.3 mL of a 0.47 N ethanolic HCl solution. The solution was stirred for 15 min at 0° C. before concentration to dryness at RT under vacuum. For analysis purpose, a small batch of (±)-canadine hydrochloride 3 was recrystallized in MeOH and the product was isolated as a white solid.

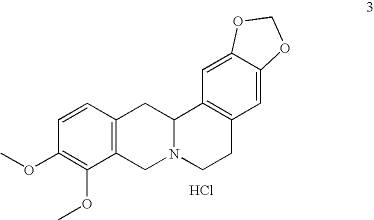

MW: 375.85; Yield: 89%; White Solid; Mp (° C.): 213.5.

R_{f}: 0.2 (cyclohexane:EtOAc=4:1, free base).

¹H-NMR (DMSO d₆, δ): 2.85-2.90 (m, 1H, CHH), 3.05 (dd, 1H, J=12.3 Hz & 16.5 Hz, CHH), 3.38-3.50 (m, 2H, CH₂), 3.72-3.82 (m, 2H, N—CH₂), 3.79 (s, 3H, O—CH₃), 3.82 (s, 3H, O—CH₃), 4.39 (d, 1H, J=15.3 Hz), 4.66-4.69 (m, 2H), 6.02-6.03 (m, 2H, OCH₂O), 6.83 (s, 1H, Ar—H), 7.01 (d, 1H, J=8.6 Hz, Ar—H), 7.08 (d, 1H, J=8.6 Hz, Ar—H), 7.09 (s, 1H, Ar—H).

¹³C-NMR (DMSO d₆, δ): 25.4, 32.1, 50.0, 51.0, 56.0, 58.9, 60.0, 101.3, 105.6, 108.3, 112.9, 122.5, 124.1, 124.8, 125.1, 125.5, 144.4, 146.7, 146.9, 150.5.

MS-ESI m/z (rel. int.): 340.0 ([MH]⁺, 100).

HPLC: Method A, detection UV 254 nm, RT=4.70 min, peak area 96.1%.

### Demethylene berberine or 9,10-dimethoxy-5,6-dihydro-isoquino[3,2-a]isoquinolinylium-2,3-diol chloride 4

To a suspension of berberine chloride (6.98 g, 18.8 mmol) in CH₂Cl₂ (155 mL) at 0° C. under nitrogen in a 500 mL round-bottomed flask equipped with a magnetic stirrer was added dropwise BCl₃ (1 N solution in CH₂Cl₂, 57.6 mL, 57.6 mmol) through a dropping funnel and the reaction mixture was stirred for 1 h at 0° C., then for 20 h at reflux. Another portion of BCl₃ (1 N solution in CH₂Cl₂, 19.0 mL, 19.0 mmol) was then added dropwise to the warm solution and the reaction mixture was stirred overnight at reflux. After cooling to RT, MeOH (100 mL) was carefully added and the volatiles were evaporated at 40° C. under vacuum. The solid was then purified by column chromatography (SiO₂; eluent CH₂Cl₂:MeOH=20:1 to 3:1). The fractions containing the pure product were combined and the solution was concentrated at 40° C. under vacuum to a volume of 50 mL. The solution was left to stand for 20 h, after which orange crystals were isolated and identified as demethylene berberine chloride 4 (2.52 g). The filtrate was concentrated to dryness at 40° C. under vacuum, affording another batch of 4 (0.97 g).

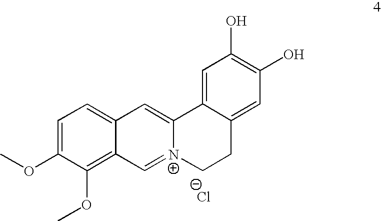

MW: 359.80; Yield: 52%; Orange Solid; Mp (° C.): 219.2.

R_{f}: 0.4 (CH₂Cl₂:MeOH=100:8).

¹H-NMR (CD₃OD, δ): 3.15-3.21 (m, 2H, CH₂), 4.10 (s, 3H, O—CH₃), 4.20 (s, 3H, O—CH₃), 4.87-4.92 (m, 2H, N—CH₂), 6.82 (s, 1H, Ar—H), 7.51 (s, 1H, Ar—H), 7.97 (d, 1H, J=8.4 Hz, Ar—H), 8.09 (d, 1H, J=8.4 Hz, Ar—H), 8.57 (s, 1H, Ar—H), 9.72 (s, 1H, Ar—H).

¹³C-NMR (CD₃OD, δ): 27.7, 57.6, 57.7, 62.5, 113.5, 115.8, 119.5, 120.6, 123.2, 124.4, 128.1, 128.8, 135.5, 140.5, 145.7, 146.2, 147.3, 150.9, 151.7.

MS-ESI m/z (rel. int.): 324.0 ([M]⁺, 100).

HPLC: Method A, detection UV 254 nm, RT=4.07 min, peak area 95.9%.

### (±)-N-benzyl canadinium or (±)-7-benzyl-9,10-dimethoxy-5,8,13,13a-tetrahydro-6H-[1,3]dioxolo[4,5-g]isoquino[3,2-a]isoquinolinylium bromide 5

A mixture of (±)-canadine (176 mg, 0.52 mmol) and benzyl bromide (1.0 mL, 8.41 mmol) was stirred for 4 h at 100° C. in a 10 mL round-bottomed flask equipped with a magnetic stirrer. After cooling, the solid was filtrated, washed several times with Et₂O (5×5 mL) and purified by column chromatography (SiO₂; eluent CH₂Cl₂:MeOH=100:6). (±)-N-Benzyl canadinium bromide 5 was isolated as a off-white solid solid (107 mg, 40% yield).

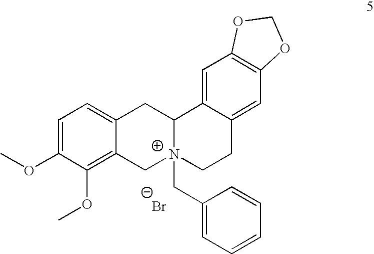

MW: 510.42; Yield: 40%; Off-white Solid; Mp (° C.): 211.5.

R_{f}: 0.3 (CH₂Cl₂:MeOH=100:6)

¹H-NMR (CDCl₃:CD₃OD=1:1, δ): 3.35-3.41 (m, 2H, CH₂), 3.57-3.70 (m, 2H, CH₂), 3.89 (s, 3H, O—CH₃), 3.97 (s, 3H, O—CH₃), 4.00-4.14 (m, 2H, N—CH₂), 4.19 (s, 2H, N—CH₂), 4.67 (d, 1H, J=16.0 Hz, N—CHH), 4.76 (d, 1H, J=16.0 Hz, N—CHH), 5.53 (dd, 1H, J=5.6 Hz & 12.1 Hz, N—CH), 6.04-6.05 (m, 2H, OCH₂O), 6.86 (s, 1H, Ar—H), 6.96 (s, 1H, Ar—H), 7.14 (d, 1H, J=8.7 Hz, Ar—H), 7.21-7.24 (m, 3H, Ar—H), 7.47-7.58 (m, 3H, Ar—H).

¹³C-NMR (CDCl₃:CD₃OD=1:1, δ): 27.1, 31.7, 54.5, 58.7, 59.2, 59.8, 63.7, 70.1, 104.7, 108.4, 111.4, 116.8, 122.6, 124.7, 125.0, 126.2, 127.5, 128.6, 132.5 (2×C), 134.0, 135.1 (2×C), 147.8, 151.0, 151.5, 154.3.

MS-ESI m/z (rel. int.): 430.1 ([MH]⁺, 100).

HPLC: Method A, detection UV 254 nm, RT=5.00 min, peak area 96.5%.

### 2,3,9,10-Tetrahydroxyberberine or 5,6-dihydro-isoquino[3,2-a]isoquinolinylium-2,3,9,10-tetraol chloride 6

To a suspension of berberine chloride (1.21 g, 3.25 mmol) in CH₂Cl₂ (55 mL) at −10° C. in a 250 mL round-bottomed flask equipped with a magnetic stirrer was added dropwise BBr₃ (1 N solution in CH₂Cl₂, 16.0 mL, 16.0 mmol) and the reaction mixture was stirred for 1 h at −10° C., then for 17 h at RT. A further portion of BBr₃ (1 N solution in CH₂Cl₂, 10.0 mL, 10.0 mmol) was then added at RT and the reaction mixture was refluxed for a further 6 h, after which it was cooled down to RT, quenched with MeOH (40 mL) and concentrated at 40° C. under vacuum. The solid was recrystallized in MeOH to give 2,3,9,10-tetrahydroxyberberine chloride 6 as a yellow solid (682 mg, 63% yield).

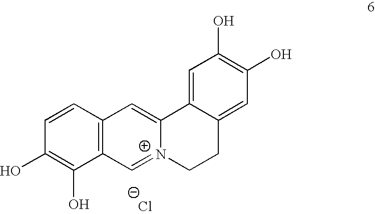

MW: 331.75; Yield: 63%; Yellow Solid; Mp (° C.): 338.8.

R_{f}: 0.3 (CH₂Cl₂:MeOH=100:17).

¹H-NMR (CD₃OD, δ): 3.15 (t, 2H, J=6.2 Hz, CH₂), 4.77-4.90 (m, 2H, N—CH₂), 6.80 (s, 1H, Ar—H), 7.44 (s, 1H, Ar—H), 7.57 (d, 1H, J=8.2 Hz, Ar—H), 7.74 (d, 1H, J=8.2 Hz, Ar—H), 8.38 (s, 1H, Ar—H), 9.63 (s, 1H, Ar—H).

¹³C-NMR (CD₃OD, δ): 26.6, 55.9, 111.7, 114.4, 117.8, 118.3, 118.4, 119.1, 126.8, 128.9, 132.8, 137.2, 142.0, 143.6, 143.8, 145.7, 148.9.

MS-ESI m/z (rel. int.): 296 ([MH]⁺, 100).

HPLC: Method A, detection UV 254 nm, RT=3.80 min, peak area 99.3%.

### 2-(2,3-Dimethoxybenzyl)-6,7-dimethoxy-1,2,3,4-tetrahydroisoquinoline hydrochloride 7

To a solution of 6,7-dimethoxy-1,2,3,4-tetrahydroisoquinoline hydrochloride (2.0 g, 8.70 mmol) and triethylamine (1.2 mL, 17.4 mmol) in THF (120 mL) was added 2,3-dimethoxybenzaldehyde (1.6 g, 7.9 mmol) in a 250 mL round-bottomed flask equipped with a magnetic stirrer. The reaction mixture was stirred for 2 h at 70° C., then at 20° C. MeOH (10 mL), AcOH (0.5 mL) and sodium triacetoxyborohydride (2.5 g, 11.8 mmol) were successively added and the reaction mixture was stirred at RT for 48 h. Water (5 mL) was added, solvents were evaporated and the crude product was partitioned between EtOAc (350 mL) and a 1 M K₂CO₃ solution (50 mL). The organic phase was washed by water (20 mL), brine (20 ml) and was evaporated to give 3.7 g of a crude pale yellow solid.

A portion of crude compound (1.0 g) was purified by column chromatography (SiO₂; eluent cyclohexane:EtOAc=92:8 to 68:32) to give after evaporation a pale yellow powder (450 mg, 56% yield). The hydrochloride salt was prepared from a portion of free base (220 mg, 0.64 mmol) using a 0.6 N HCl solution in MeOH (1.6 mL, 0.96 mmol) to give after evaporation and drying 2-(2,3-dimethoxybenzyl)-6,7-dimethoxy-1,2,3,4-tetrahydro isoquinoline hydrochloride 7 (235 mg, 54% yield) as a white solid.

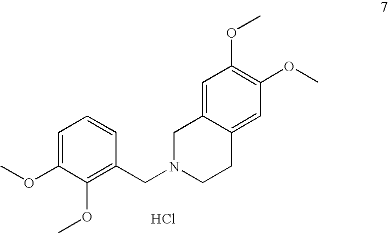

MW: 379.88; Yield: 54%; White Solid; Mp (° C.): 132.5.

R_{f}: 0.18 (cyclohexane:EtOAc=7:3, free base).

¹H-NMR (CD₃OD, δ): 3.00-3.23 (m, 2H, CH₂), 3.35-3.47 (m, 1H, N—CH₂), 3.65-3.77 (m, 1H, N—CH₂), 3.78 (s, 3H, O—CH₃), 3.81 (s, 3H, O—CH₃), 3.91 (s, 3H, O—CH₃), 3.94 (s, 3H, O—CH₃), 4.32 (s, 2H, N—CH₂), 4.47 (s, 2H, N—CH₂), 6.73 (s, 1H, Ar—H), 6.81 (s, 1H, Ar—H), 7.09 (dd, 1H, J=6.2 Hz, J=2.7 Hz, Ar—H), 7.14-7.25 (m, 2H, Ar—H).

¹³C-NMR (CD₃OD, δ): 25.8, 51.0, 53.8, 55.4, 56.4, 56.5, 56.6, 61.6, 110.9, 112.7, 116.3, 120.6, 123.8, 124.3, 124.8, 125.8, 149.8, 150.0, 150.8, 154.3.

MS-ESI m/z (rel. int.): 344.0 ([MH]⁺, 100), 150.9 (10).

HPLC: Method A, detection UV 282 nm, R_{T}=4.3 min, peak area 96.0%.

### Preparation of Compound 12

### (±)-8-Benzyl-9,10-dimethoxy-5,8,13,13a-tetrahydro-6H-[1,3]dioxolo[4,5-g]isoquino[3,2-a]isoquinoline hydrochloride 12

(±)-8-Benzyl-9,10-dimethoxy-5,8,13,13a-tetrahydro-6H-[1,3]dioxolo[4,5-g]isoquino[3,2-a]isoquinoline (Sigma-Aldrich (St. Louis, Mo., USA), 28.8 mg, 0.067 mmol). was dissolved in MeOH (5 mL) in a 10 mL round-bottomed flask equipped with a magnetic stirrer and the solution was cooled to 0° C. in an ice bath before adding 0.77 mL of a 0.13 N HCl solution in MeOH. The solution was stirred for 15 min at 0° C. before concentration to dryness at RT under vacuum. The solid obtained was dissolved in DMSO (1 mL) and purified using reversed phase HPLC on C18 Xterra Column 19×50 mm, 5 μm part 186001108 with a gradient of 0 to 30% CH₃CN (0.05% TFA) in H₂O (0.05% TFA) in 7 min. After 8 injections all the selected fraction were combined and evaporated under reduced pressure to give the desired product (15 mg) which was dissolved in MeOH (1 mL) in a 10 mL round-bottomed flask equipped with a magnetic stirrer and the solution was cooled to 0° C. in an ice bath before adding 0.422 mL of a 0.13 N HCl solution in MeOH. After evaporation and drying (±)-8-benzyl-9,10-dimethoxy-5,8,13,13a-tetrahydro-6H-[1,3]dioxolo[4,5-g]isoquino[3,2-a]isoquinoline (12 mg, 38% yield) was obtained as a pale brown solid.

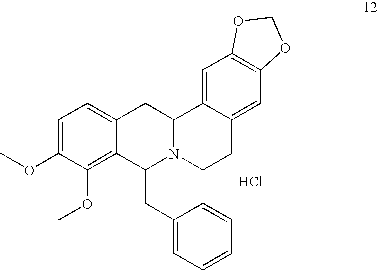

MW: 466.0; Pale Brown Solid; Yield: 38%; Mp (° C.)=196.9.

¹H-NMR (CD₃OD, δ): 3.05-3.50 (m, 6H, 3×CH₂), 3.60-3.75 (m, 2H, CH₂), 3.90 (s, 3H, OMe), 4.04 (s, 3H, OMe), 4.54 (d, 1H, J=10.9 Hz, OCH), 5.09 (d, 1H, J=9.25 Hz, OCH), 5.97 (s, 2H, CH₂), 6.68 (s, 1H, ArH), 7.04 (s, 1H, ArH), 7.12 (s, 2H, ArH), 7.33-7.61 (m, 5H, ArH).

¹³C-NMR (CDCl₃, δ): 27.6, 33.0, 43.0, 54.6, 56.6, 61.2, 64.1, 68.9, 103.1, 106.8, 109.3, 114.6, 125.1, 125.6, 126.3, 126.6, 127.5, 129.0, 129.8 (2×C), 130.6 (2×C), 138.8, 146.6, 149.1, 149.5, 153.1.

MS-ESI m/z (% rel. Int.): 430.1 ([MH]⁺, 100).

HPLC: Method A, detection at 280 nm, RT=6.13 min, peak area 93%.

### Preparation of Compound 15

### 8-Methyl-isoquino[3,2-a]isoquinolinylium-2,3,10,11-tetraol chloride 15

To a suspension of coralyne chloride hydrate 8 (723 mg, 1.73 mmol) in CH₂Cl₂ (20 mL) at −78° C. in a 250 mL round-bottomed flask equipped with a magnetic stirrer was added dropwise BBr₃ (1 N solution in CH₂Cl₂, 10.5 mL, 10.5 mmol) and the reaction mixture was stirred for 1 h at −78° C., then for 2 days at RT. The reaction medium was cooled down to 0° C. in an ice bath, quenched with MeOH (15 mL) and adjusted to pH=2 with a 2 N aqueous solution of HCl. The volatiles were removed under vacuum at 40° C. and the resulting solid was purified by column chromatography (SiO₂; eluent CH₂Cl₂:MeOH=100:17 to 100:35). 8-Methyl-isoquino[3,2-a]isoquinolinylium-2,3,10,11-tetraol chloride 15 was isolated as a yellow solid (0.59 g, 99% yield). For analysis purpose, a small batch of 15 was purified by preparative HPLC.

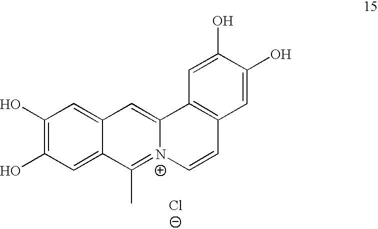

MW: 343.76; Yield: 99%; Yellow Solid; Mp (° C.)>270 (dec.).

R_{f}: 0.2 (CH₂Cl₂:MeOH=100:50).

¹H-NMR (DMSO d₆, δ): 3.21 (s, 3H, CH₃), 7.38 (s, 1H, Ar—H), 7.60 (s, 1H, Ar—H), 7.74-7.77 (m, 1H, Ar—H), 7.82 (s, 1H, Ar—H), 8.26 (s, 1H, Ar—H), 8.55-8.59 (m, 1H, Ar—H), 9.20 (s, 1H, Ar—H), 4 OH not seen.

¹³C-NMR (DMSO d₆, δ): 16.9, 107.2, 107.9, 109.3, 111.5, 114.5, 119.2, 120.2, 121.5, 122.3, 123.2, 133.2, 133.9, 143.9, 149.3, 150.3, 151.5, 155.7.

MS-ESI m/z (rel. int.): 308 ([M]⁺, 100).

HPLC: Method A, detection UV 254 nm, RT=3.78 min, peak area 99.9%.

### Preparation of Compound 16

### (±)-5,8,13,13a-Tetrahydro-6H-isoquino[3,2-a]isoquinolinylium-2,3,9,10-tetraol hydrochloride 16

To a suspension of 2,3,9,10-tetrahydroxyberberine 6 (350 mg, 1.06 mmol) in MeOH (30 mL) was slowly added NaBH₄ (160 mg, 4.23 mmol) in a 100 mL round-bottomed flask equipped with a magnetic stirrer. The reaction mixture was stirred at RT for 0.5 h, after which the pH was adjusted at 2 with a 1 N aqueous solution of HCl. The volatiles were then removed at 40° C. under vacuum. The resulting mixture was taken up in n-BuOH (30 mL) and the solution was washed with water (3×15 mL), brine (10 mL), dried (Na₂SO₄) and concentrated at 40° C. under vacuum. The solid was then dissolved in hot MeOH (80 mL), precipitated with Et₂O (300 mL) and filtered. The isolated powder was recrystallized from H₂O to afford (±)-5,8,13,13a-tetrahydro-6H-isoquino[3,2-a]isoquinolinylium-2,3,9,10-tetraol hydrochloride 16 as a brown solid (250 mg, 71% yield). For analysis purpose, a small batch 16 of was purified by preparative HPLC.

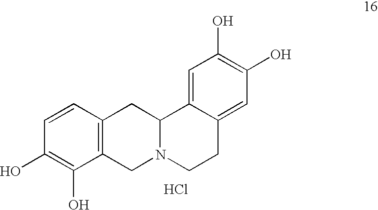

MW: 299.32; Yield: 71%; Brown Solid; Mp (° C.)>270 (dec.).

R_{f}: 0.2 (CH₂Cl₂:MeOH=100:10).

¹H-NMR (DMSO d₆, δ): 2.77 (d, 1H, J=5.0 Hz), 2.90-3.00 (m, 2H), 3.50 (dd, 1H, J=0.8 and 5.0 Hz), 3.77-3.79 (m, 1H), 4.11-4.21 (m, 2H), 4.53 (d, 1H, J=4.7 Hz, Ar—H), 4.52-4.64 (m, 1H), 6.59 (s, 1H, Ar—H), 6.60 (d, 1H, J=2.2 Hz, Ar—H), 6.78 (s, 1H, Ar—H), 6.79 (d, 1H, J=2.2 Hz, Ar—H), 8.99, 9.02, 9.26, 9.47 (4 s, 4H, 4×OH), 10.86 (br, s, 1H, NH).

¹³C-NMR (DMSO d₆, δ): 24.7, 32.4, 50.4, 51.3, 58.8, 112.3, 115.0, 115.1, 116.4, 118.9, 122.2, 122.5, 122.7, 141.2, 143.1, 144.6, 145.1.

MS-ESI m/z (rel. int.): 300 ([MH]⁺, 100).

HPLC: Method A, detection UV 283 nm, RT=3.33 min, peak area 99.3%.

### Preparation of Compounds 17 to 19

### (±)-9,10-Dimethoxy-5,8,13,13a-tetrahydro-6H-isoquino[3,2-a]isoquinoline-2,3-diol hydrochloride 17

To a suspension of demethylene berberine chloride 4 (351 mg, 0.98 mmol) in MeOH (30 mL) was slowly added NaBH₄ (148 mg, 3.91 mmol) in a 100 mL round-bottomed flask equipped with a magnetic stirrer. The reaction mixture was stirred at RT for 1 h, after which the pH was adjusted to pH=2 with 1N aqueous HCl solution and the mixture was concentrated to dryness at 40° C. under vacuum.

A solution of the above salt (51 mg, 0.14 mmol) in acetic anhydride (15 mL) was stirred under reflux for 2 h in a 100 mL round-bottomed flask equipped with a magnetic stirrer. Excess of acetic anhydride was then removed under vacuum and the remaining oil was taken up in CH₂Cl₂ (30 mL). Water (6 mL) was then added and the pH adjusted to pH=12 with 2N aqueous solution of NaOH. The organic phase was isolated and the aqueous phase further extracted with CH₂Cl₂ (2×10 mL). The organic phases were combined, washed with brine (10 mL), dried and concentrated under vacuum. Purification by column chromatography (SiO₂; eluent EtOAc) gave after evaporation a yellow solid (39 mg) that was immediately dissolved in acetone (6 mL) in a 50 mL round-bottomed flask equipped with a magnetic stirrer. 3N Aqueous HCl solution (4 mL) was then added dropwise and the mixture was stirred for 40 h under reflux. After cooling to RT, the mixture was concentrated to dryness to give a solid that was washed with EtOAc and recrystallized from MeOH/Et₂O. (±)-9,10-Dimethoxy-5,8,13,13a-tetrahydro-6H-isoquino[3,2-a]isoquinoline-2,3-diol hydrochloride 17 was obtained as a pale brown solid (14 mg, 27% yield).

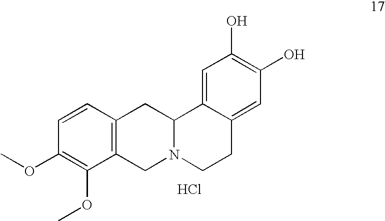

MW: 363.84; Yield: 27%; Pale Brown Solid; Mp (° C.)>251 (dec.).

R_{f}: 0.25 (CH₂Cl₂:MeOH=100:5).

¹H-NMR (CD₃OD d₄, δ): 2.86-3.26 (m, 3H), 3.47-3.57 (m, 1H), 3.66-3.72 (m, 1H), 3.83-3.97 (m, 1H), 3.88 (s, 3H, OCH₃), 3.90 (s, 3H, OCH₃), 4.43 (d, 1H, J=15.9 Hz), 4.67 (dd, 1H, J=4.2 and 12.0 Hz), 4.75 (d, 1H, J=15.9 Hz), 6.66 (s, 1H, Ar—H), 6.80 (s, 1H, Ar—H), 7.07 (s, 2H, Ar—H).

¹³C-NMR (DMSO d₆, δ): 26.5, 33.9, 50.1, 53.0, 56.5, 60.9, 61.1, 113.1, 114.5, 116.1, 122.6, 123.2, 123.8, 125.1, 125.3, 146.4 (2×C), 147.0, 152.4.

MS-ESI m/z (rel. int.): 328 ([M+H]⁺, 100).

HPLC: Method A, detection UV 254 nm, RT=5.15 min, peak area 98.4%.

### 2-(2,3-Dihydroxybenzyl)-6,7-dimethoxy-1,2,3,4-tetrahydroisoquinolinium chloride 18

To a stirred solution of 6,7-dimethoxy-1,2,3,4-tetrahydroisoquinoline hydrochloride (600 mg, 2.61 mmol) in THF (30 mL) was added Et₃N (730 uL, 5.22 mmol) and 2,3-dihydroxybenzaldehyde (410 mg, 2.90 mmol) under an nitrogen atmosphere. The mixture was stirred at RT for 0.5 h and AcOH (250 uL) and sodium triacetoxyborohydride (720 mg, 3.40 mmol) were added portionwise. The mixture was stirred at RT for 72 h. Water (1 mL) was added and the solvents were evaporated at 40° C. The crude product was partitioned between a 1M K₂CO₃ aqueous solution (50 mL) and EtOAc (100 mL) to give a precipitate of potassium salt (470 mg, 46%) which was filtered. This product was dissolved in MeOH (5 mL) with 0.47N HCl solution in MeOH (5.1 mL, 2.4 mmol), filtered and evaporated to give after drying under vacuum a crude hydrochlorid salt. This solid was dissolved in MeOH (20 mL) then a 7N ammonia solution in MeOH (400 uL, 2.8 mmol) was added and the precipitate was filtered, washed successively with MeOH and water, dried under vacuum to give 2-(2,3-dihydroxybenzyl)-6,7-dimethoxy-1,2,3,4-tetrahydroisoquinoline (237 mg, 29% yield).

Finally the above compound (237 mg 0.75 mmol) was stirred in a mixture of water (2.0 mL) and a 6 M HCl solution (125 uL, 0.75 mmol) for 5 min at RT to give after evaporation and drying under vacuum 2-(2,3-dihydroxybenzyl)-6,7-dimethoxy-1,2,3,4-tetrahydroisoquinolinium chloride 18 as a beige solid (245 mg, 27% yield).

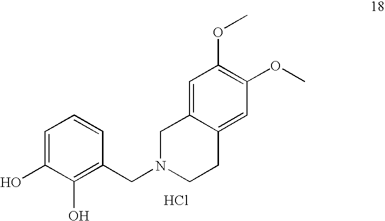

MW: 351.82; Yield: 27%; Beige solid; Mp (° C.): 237.2.

R_{f}: (free base): 0.70 (EtOAc).

¹H-NMR (CD₃OD, δ): 3.08-3.18 (m, 2H, Ar—CH₂), 3.33-3.45 (m, 1H, N—CH₂), 3.71-3.79 (m, 1H, N—CH₂), 3.79 (s, 3H, OMe), 3.81 (s, 3H, OMe), 4.31 (d, 1H, N—CH₂), 4.38 (d, 1H, N—CH₂), 4.45 (s, 2H, N—CH₂), 6.74-6.80 (m, 3H, Ar—H), 6.86-6.94 (m, 2H, Ar—H).

¹³C-NMR (CD₃OD, δ): 25.8, 50.7, 53.7, 55.7, 56.5, 56.5, 110.9, 112.7, 117.2, 118.1, 120.7, 121.1, 123.9, 124.4, 146.7, 146.8, 149.9, 150.7.

MS-ESI m/z (% rel. Int.): 194.1 (15), 316.1 ([MH]⁺, 100).

HPLC: Method A, detection UV 254 nm, RT=3.90 min, peak area 99%.

### 2-(2,3-Dihydroxybenzyl)-6,7-dihydroxy-1,2,3,4-tetrahydroisoquinolinium chloride 19

To a stirred solution of 2-(2,3-dimethoxybenzyl)-6,7-dimethoxy-1,2,3,4-tetrahydroisoquinoline 7 (200 mg, 0.58 mmol) in CH₂Cl₂ (35 mL) at −78° C. under nitrogen was added dropwise a 1M BBr₃ solution in CH₂Cl₂ (4.7 mL, 4.7 mmol). The mixture was stirred at −78° C. for 10 min then 15 h at RT. MeOH (1.5 mL) was slowly added at +4° C. and the solvents were evaporated at 30° C. The crude product was dried under vacuum for 1 h then was dissolved in water (5 mL). An aqueous solution of 20% ammonia (70 uL, 1.26 mmol) was added until to obtain a precipitate (pH=7). The solution was extracted with n-butanol (2×25 mL) and the organic layer was evaporated at 70° C. and dried under vacuum for 72 h to give 2-(2,3-dihydroxybenzyl)-1,2,3,4-tetrahydroisoquinoline-6,7-diol as a white solid (153 mg).

This compound was dissolved in MeOH (4.0 mL) and treated with a 0.47M HCl solution in MeOH (1.0 mL, 0.47 mmol) to give after evaporation at 20° C. and drying under vacuum a pale yellow solid. This solid was stirred in pentane (10 mL) overnight at 20° C., filtered under nitrogen atmosphere for 3 h to give 2-(2,3-dihydroxybenzyl)-6,7-dihydroxy-1,2,3,4-tetrahydroisoquinolinium chloride 19 as a white solid (102.3 mg, 54% yield).

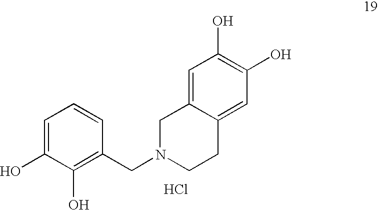

MW: 323.77; Yield: 54%; White solid; Mp (° C.): 178.8-178.8.

R_{f}: (free base): (CH₂Cl₂:MeOH=99:1).

¹H-NMR (CD₃OD, δ): 3.00-3.02 (t, 2H, J=7.8 Hz, Ar—CH₂), 3.35 (s, 2H, N—CH₂), 4.26 (s, 2H, N—CH₂), 4.42 (s, 2H, N—CH₂—), 6.54 (s, 1H, Ar—H), 6.62 (s, 1H, Ar—H), 6.77 (t, 1H, J=7.7 Hz, Ar—H), 6.85 (dd, 1H, J=7.7 Hz, J=1.5 Hz, Ar—H), 6.92 (dd, 1H, J=7.8 Hz, J=1.5 Hz, Ar—H).

¹³C-NMR (CD₃OD, δ): 25.6, 50.8, 53.9, 55.6, 114.0, 115.9, 117.2, 118.1, 119.4, 121.0, 123.0, 123.8, 146.0, 146.6, 146.8, 147.0.

MS-ESI m/z (% rel. Int.): 288.0 ([MH]⁺, 100).

HPLC: Method A, detection UV 254 nm, RT=0.86 min, peak area 96%.

### Preparation of Compound 20

### (±)-Ethyl 5-(2-(chloromethyl)-3,4-dimethoxybenzyl)-7,8-dihydro-[1,3]dioxolo[4,5-g]isoquinoline-6(5H)-carboxylate CCH 16156a

A solution of (±)-tetrahydroberberine hydrochloride 3 (1.20 g, 3.54 mmol) in ethyl chloroformate (100 mL) was stirred for 2 days under reflux in a 250 mL round-bottomed flask equipped with a magnetic stirrer. Excess of ethyl chloroformate was then removed under vacuum and the residue was taken up in CH₂Cl₂ (60 mL). The solution was washed with 1N aqueous K₂CO₃ (15 mL), brine (10 mL), dried over Na₂SO₄ and concentrated under vacuum. The crude oil was finally purified by column chromatography (SiO₂; eluent cyclohexane:EtOAc=5:1) to give, after evaporation and drying under high vacuum, ethyl (±)-5-(2-(chloromethyl)-3,4-dimethoxybenzyl)-7,8-dihydro-[1,3]dioxolo[4,5-g]isoquinoline-6(5H)-carboxylate CCH 16156a as a colorless oil (200 mg, 15% yield).

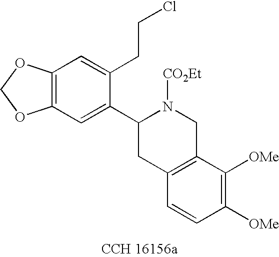

MW: 447.91; Yield: 15%; Colorless oil.

R_{f}: (free base): 0.3 (cyclohexane:EtOAc=3:1).

¹H-NMR (CDCl₃, δ): 1.15-1.26 (m, 3H, CH₃), 2.84 (dd, 1H, J=4.9 and 15.7 Hz), 3.09-3.17 (m, 2H), 3.23 (dd, 1H, J=6.2 and 15.7 Hz), 3.62-3.82 (m, 2H, CH₂Cl), 3.86 (s, 6H, 2×OCH₃), 4.13 (q, 2H, J=7.1 Hz, OCH₂CH₃), 4.20 (d, 1H, J=16.5 Hz), 5.09 (d, 1H, J=16.5 Hz), 5.33-5.46 (m, 1H), 5.84 and 5.85 (2d, 2H, J=1.4 Hz, OCH₂O), 6.49 (s, 1H, Ar—H), 6.66 (s, 1H, Ar—H), 6.80 (d, 1H, J=8.3 Hz, Ar—H), 6.86 (d, 1H, J=8.3 Hz, Ar—H).

¹³C-NMR (CDCl₃, δ): 14.7, 34.1, 35.8, 39.2, 44.3, 51.1, 55.9, 60.7, 61.7, 101.0, 106.8, 110.1, 111.3, 123.3, 126.8, 128.3, 129.0, 134.3, 145.0, 146.5 (2C), 151.1, 155.6.

MS-ESI m/z (rel. int.): 448 ([M+H]⁺, 53), 470 ([M+Na]⁺, 47).

HPLC: Method A, detection UV 254 nm, RT=7.15 min.

### (±)-Ethyl 7,8-dimethoxy-3-(6-vinylbenzo[d][1,3]dioxol-5-yl)-3,4-dihydro isoquinoline-2(1H)-carboxylate CCH 16190

To a solution of CCH 16156a (81 mg, 181 μmol) in EtOH (2 mL) in a 10 mL round-bottomed flask equipped with a magnetic stirrer was added 2N aqueous NaOH (2 mL) and the mixture was stirred overnight under reflux. After cooling to RT, EtOH was removed under vacuum and the solution was extracted with CH₂Cl₂ (2×5 mL). The organic phase was washed with brine (5 mL), dried over Na₂SO₄, filtered and concentrated under vacuum. The resulting oil was finally purified by column chromatography (SiO₂; eluent cyclohexane:EtOAc=5:1) to give, after evaporation and drying, (±)-ethyl 7,8-dimethoxy-3-(6-vinylbenzo[d][1,3]dioxol-5-yl)-3,4-dihydroisoquinoline-2(1H)-carboxylate CCH 16190 as a colorless oil (63 mg, 85% yield).

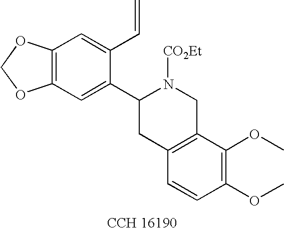

MW: 411.45; Yield: 85%; Colorless oil.

R_{f}: 0.3 (cyclohexane:EtOAc=5:1)

¹H-NMR (CDCl₃, δ): 1.19-1.29 (m, 3H, CH₃), 2.84 (dd, 1H, J=4.5 and 15.5 Hz), 3.18 (dd, 1H, J=5.7 and 15.5 Hz), 3.85 (s, 6H, 2×OCH₃), 4.08-4.24 (m, 3H), 5.04 (d, 1H, J=16.5 Hz), 5.24 (dd, 1H, J=1.3 and 10.9 Hz), 5.51 (dd, 1H, J=1.3 and 17.0 Hz), 5.30-5.57 (m, 1H), 5.86 (s, 2H, OCH₂O), 6.46 (s, 1H, Ar—H), 6.77 (d, 1H, J=8.3 Hz, Ar—H), 6.83 (d, 1H, J=8.3 Hz, Ar—H), 6.94 (s, 1H, Ar—H), 7.06 (dd, 1H, J=10.9 and 17.0 Hz).

MS-ESI m/z (rel. int.): 412 ([M+H]⁺, 33), 434 ([M+Na]⁺, 44), 845 ([2M+Na]⁺, 23).

HPLC: Method A, detection UV 254 nm, RT=7.49 min.

### (±)-Ethyl 3-(6-ethylbenzo[d][1,3]dioxol-5-yl)-7,8-dimethoxy-3,4-dihydroisoquinoline-2(1H)-carboxylate CCH 16192

To a solution of CCH 16190 (115 mg, 279 μmol) in CH₂Cl₂:MeOH=1:1 (5 mL) in a 50 mL round-bottomed flask equipped with a magnetic stirrer was added Pd/C (10 wt. %, 30 mg) and the mixture was stirred for 5 h under H₂ (1 atm.). The catalyst was then removed by filtration through celite and the filtrate was concentrated to dryness. The resulting oil was finally purified by column chromatography (SiO₂; eluent cyclohexane:EtOAc=4:1) to give, after evaporation and drying, (±)-ethyl 3-(6-ethylbenzo[d][1,3]dioxol-5-yl)-7,8-dimethoxy-3,4-dihydroisoquinoline-2(1H)-carboxylate CCH 16192 as a colorless oil (100 mg, 87% yield).

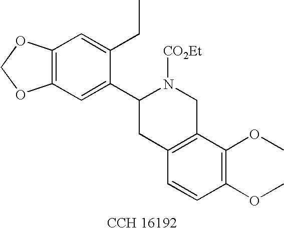

MW: 413.46; Yield: 87%; Colorless oil.

R_{f}: 0.3 (cyclohexane:EtOAc=4:1).

¹H-NMR (CDCl₃, δ): 1.18-1.30 (m, 6H, 2×CH₃), 2.68 (q, 2H, J=7.5 Hz, CH₂CH₃), 2.84 (dd, 1H, J=4.7 and 15.6 Hz), 3.22 (dd, 1H, J=6.1 and 15.6 Hz), 3.85 (s, 6H, 2×OCH₃), 4.09 (q, 2H, J=7.1 Hz, OCH₂CH₃), 4.20 (d, 1H, J=16.7 Hz), 5.09 (d, 1H, J=16.7 Hz), 5.36-5.52 (m, 1H, CH), 5.81 (s, 2H, OCH₂O), 6.46 (s, 1H, Ar—H), 6.65 (s, 1H, Ar—H), 6.79 (d, 1H, J=8.3 Hz, Ar—H), 6.85 (d, 1H, J=8.3 Hz, Ar—H).

¹³C-NMR (CDCl₃, δ): 14.6, 15.6, 25.3, 34.3, 39.2, 51.0, 55.8, 60.7, 61.4, 100.7, 106.4, 108.9, 111.2, 123.3, 127.2, 128.5, 133.1, 135.3, 145.0, 145.3, 146.4, 151.0, 155.7.

MS-ESI m/z (rel. int.): 414 ([M+H]⁺, 27), 426 ([M+Na]⁺, 53), 849 ([2M+Na]⁺, 20)

HPLC: Method A, detection UV 254 nm, RT=7.67 min.

### (±)-3-(6-Ethylbenzo[d][1,3]dioxol-5-yl)-7,8-dimethoxy-2-methyl-1,2,3,4-tetrahydroisoquinolinylium chloride 20

LiAlH₄ (47 mg, 1.24 mmol) was added to a solution of CCH 16192 (100 mg, 0.242 mmol) in anhydrous Et₂O (15 mL) in a 100 mL round-bottomed flask equipped with a magnetic stirrer at RT under N₂ and the mixture was stirred under reflux for 3 h after which it was cooled down to RT. The reaction was then quenched with 2N aqueous NaOH solution (10 mL). The organic phase was isolated and the aqueous phase further extracted with EtOAc. The organic phases were combined, washed with brine (8 mL), dried (Na₂SO₄) and concentrated under vacuum. Purification by column chromatography (SiO₂; eluent cyclohexane:EtOAc=5:1) gave a colorless oil (76 mg, 88% yield).

The oil was dissolved in MeOH (3 mL) in a 25 mL round-bottomed flask equipped with a magnetic stirrer and the solution was cooled to 0° C. in an ice bath before adding 0.7 mL of a 0.47 N HCl solution in EtOH. The solution was stirred for 0.4 h at 0° C. before concentration to dryness at RT under vacuum to obtain (±)-3-(6-ethylbenzo[d][1,3]dioxol-5-yl)-7,8-dimethoxy-2-methyl-1,2,3,4-tetrahydroisoquinolinylium chloride 20 as an off-white solid.

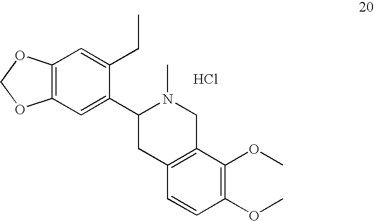

MW: 391.89; Yield: 88% (free base); Off-white Solid; Mp (° C.): 249 (dec.).

R_{f}: (free base): 0.25 (cyclohexane:EtOAc=5:1).

¹H-NMR (DMSO d₆, δ): 1.08 (t, 3H, J=7.5 Hz, CH₂CH₃), 2.53-2.64 (m, 1H), 2.57 (d, 3H, J=4.7 Hz, NHCH₃), 2.77-2.84 (m, 1H), 3.02 (dd, 1H, J=3.4 and 17.3 Hz), 3.40-3.46 (m, 1H), 3.80 (s, 3H, OCH₃), 3.82 (s, 3H, OCH₃), 4.57-4.71 (m, 3H), 6.05 (d, 1H, J=2.3 Hz, OCH₂O), 6.89 (s, 1H, Ar—H), 6.95 (d, 1H, J=8.5 Hz, Ar—H), 7.05 (d, 1H, J=8.5 Hz, Ar—H), 7.60 (s, 1H, Ar—H), 11.82 (br, s, 1H, NH).

¹³C-NMR (DMSO d₆, δ): 16.3, 25.1, 34.7, 39.6, 52.1, 55.9, 59.9, 60.2, 101.3, 106.8, 109.3, 112.6, 122.8, 123.4, 125.0, 125.7, 137.2, 144.3, 146.3, 147.7, 150.4.

MS-ESI m/z (rel. int.): 356 ([M+H]⁺, 100).

HPLC: Method A, detection UV 254 nm, RT=4.79 min, peak area 96.0%.

### Preparation of Compounds 21 to 50

### Methyl 2-(3,4-dimethoxyphenyl)acetate CCH 18056

To a solution of 3,4-dimethoxyphenylacetic acid (25.0 g, 127.4 mmol) in MeOH (100 mL) in a 500 mL round-bottomed flask equipped with a magnetic stirrer was added a catalytic amount of H₂SO₄ (a few drops) and the mixture was stirred under reflux overnight. MeOH was then removed under vacuum, then the product was taken up in CH₂Cl₂ (100 mL) and washed several times with water (4×25 mL), brine (25 mL), dried over Na₂SO₄, filtered and concentrated under vacuum to obtain methyl 2-(3,4-dimethoxyphenyl)acetate CCH 18056 as an orange oil (24.4 g, 91% yield).

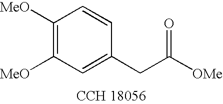

MW: 210.23; Yield: 91%; Orange oil.

R_{f}: 0.25 (cyclohexane:EtOAc=3:1).

¹H-NMR (CDCl₃, δ): 3.56 (s, 2H, CH₂), 3.70 (s, 3H, CH₃), 3.87 (s, 3H, OCH₃), 3.88 (s, 3H, OCH₃), 6.82-6.83 (m, 3H, 3×Ar—H).

¹³C-NMR (CDCl₃, δ): 40.6, 51.9, 55.8 (2×C), 111.2, 112.4, 121.4, 126.4, 148.2, 148.9, 172.2.

MS-ESI m/z (rel. int.): 233 ([M+Na]⁺, 100).

HPLC: Method A, detection UV 254 nm, RT=4.78 min.

### 6,7-Dimethoxy-1-methylisoquinolin-3-ol CCH 18060

To a solution of methyl 2-(3,4-dimethoxyphenyl)acetate CCH 18056 (23.82 g, 113.30 mmol) in acetic anhydride (57 mL) at 0° C. in a 1 L round-bottomed flask equipped with a magnetic stirrer under N₂ was added perchloric acid (70% solution in water, 11.3 mL) over a period of 30 min. The reaction mixture was then allowed to warm up to RT, stirred for a further 45 min and diluted with Et₂O (450 mL). The solid was then filtered and washed several times with Et₂O (6×15 mL) to give after drying under vacuum a dark yellow solid (27.97 g, 74% yield).

To a suspension of the above solid (11.09 g, 34.58 mmol) in H₂O (60 mL) in a 500 mL 3-neck round-bottomed flask equipped with a dropping funnel and a magnetic stirrer in an ice bath was added dropwise conc. NH₄OH (90 mL) and the reaction mixture was stirred at RT for 1 h, after which the solid was filtered and washed with cold water (4×15 mL). After drying under high vacuum, 6,7-dimethoxy-1-methylisoquinolin-3-ol CCH 18060 was isolated as a yellow solid (7.53 g, 99% yield).

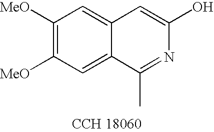

MW: 219.24; Yield: 74%; Yellow solid; Mp (° C.): 283 (dec.).

R_{f}: 0.2 (cyclohexane:EtOAc=2:1).

¹H-NMR (DMSO d₆, δ): 2.68 (s, 3H, CH₃), 3.85 (s, 3H, OCH₃), 3.86 (s, 3H, OCH₃), 6.51 (s, 1H, Ar—H), 6.97 (s, 1H, Ar—H), 7.12 (s, 1H, Ar—H), 10.69 (br, s, 1H, OH).

¹³C-NMR (DMSO d₆, δ): 20.3, 55.6, 55.7, 99.5, 103.6, 103.8, 116.2, 138.0, 147.1, 152.0, 153.3, 159.0.

MS-ESI m/z (rel. int.): 220 ([M+H]⁺, 100).

HPLC: Method A, detection UV 254 nm, RT=3.83 min.

### 6,7-Dimethoxy-1-methylisoquinolin-3-yl trifluoromethanesulfonate CCH 18064

A suspension of CCH 18060 (2.28 g, 10.40 mmol) in DMSO (100 mL) was heated in a 250 mL round-bottomed flask equipped with a magnetic stirrer until complete dissolution, then cooled down to RT before adding triethylamine (3.30 mL, 23.68 mmol) and N-phenyl-bis(trifluoromethanesulfonimide) (2.94 g, 8.23 mmol). The mixture was stirred overnight at RT, then diluted with Et₂O (100 mL) and washed with water (3×20 mL). The aqueous phase was further extracted with Et₂O (2×20 mL) and the organic phase combined, washed with brine (15 mL), dried over Na₂SO₄, filtered and concentrated under vacuum. This gave a yellow solid (3.00 g, 82% crude yield) that was used in the next step without any further purification.

For analysis purpose a small portion was purified by column chromatography (SiO₂; eluent cyclohexane:EtOAc=3:1) then recrystallized from diisopropyl ether to afford, after filtration and drying, 6,7-dimethoxy-1-methylisoquinolin-3-yl trifluoromethanesulfonate CCH 18064 as colorless needles.

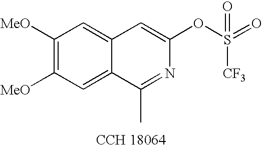

MW: 351.30; Crude Yield: 82%; Colorless needles; Mp (° C.): 153 (dec.).

R_{f}: 0.4 (cyclohexane:EtOAc=2:1).

¹H-NMR (CDCl₃, δ): 2.85 (s, 3H, CH₃), 4.03 (s, 3H, OCH₃), 4.04 (s, 3H, OCH₃), 7.06 (s, 1H, Ar—H), 7.23 (s, 1H, Ar—H), 7.25 (s, 1H, Ar—H).

¹³C-NMR (CDCl₃, δ): 22.0, 56.1, 56.2, 103.7, 105.3, 107.6, 118.8 (q, J=320.5 Hz), 123.3, 135.4, 150.6 (2C), 153.7, 156.5.

MS-ESI m/z (rel. int.): 352 ([M+H]⁺, 100).

HPLC: Method A, detection UV 254 nm, RT=6.30 min.

### 3-(Benzo[d][1,3]dioxol-5-yl)-6,7-dimethoxy-1-methylisoquinoline hydrochloride 21

To a mixture of 6,7-dimethoxy-1-methylisoquinolin-3-yl trifluoromethanesulfonate CCH 18064 (423 mg, 1.204 mmol) and 3,4-(methylenedioxy)phenylboronic acid (200 mg, 1.205 mmol) in toluene (15 mL) in a 30 mL sealed tube equipped with a magnetic stirrer was added 2N aqueous Na₂CO₃ (3.6 mL) and the reaction mixture was stirred at RT for 5 min.

[1,1′-bis(diphenylphosphino)ferrocene]dichloropalladium(II) complex (84 mg, 0.103 mmol) was then added and the mixture was stirred overnight at 80° C. The reaction mixture was cooled down to RT, diluted with EtOAc (15 mL) and the aqueous phase was removed. The organic phase was stirred in presence of charcoal (one spatula) and MgSO₄ for 30 min then filtered through celite, which gave after concentration under vacuum 0.46 g of a brown solid. Purification by column chromatography (SiO₂; eluent cyclohexane:EtOAc=3:1) afforded 3-(benzo[d][1,3]dioxol-5-yl)-6,7-dimethoxy-1-methylisoquinoline CCH 18068 as a white solid (0.24 g, 62% yield).

The solid CCH 18068 was then dissolved in MeOH (7 mL) in a 25 mL round-bottomed flask equipped with a magnetic stirrer and the solution was cooled to 0° C. in an ice bath before adding 2.4 mL of a 0.47 N HCl solution in EtOH. The solution was stirred for 0.4 h at 0° C. before concentration to dryness at RT under vacuum to obtain 3-(benzo[d][1,3]dioxol-5-yl)-6,7-dimethoxy-1-methylisoquinoline hydrochloride 21 as an off-white solid.

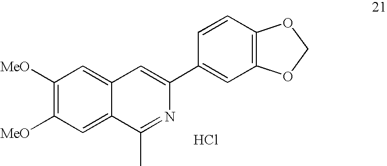

MW: 359.80; Yield: 62% (free base); Off-white solid; Mp (° C.): 217 (dec.).

R_{f}: (free base): 0.25 (cyclohexane:EtOAc=3:1).

¹H-NMR (CD₃OD, δ): 3.22 (s, 3H, CH₃), 4.13 (s, 3H, OCH₃), 4.15 (s, 3H, OCH₃), 6.11 (s, 2H, OCH₂O), 7.05 (d, 1H, J=7.9 Hz, Ar—H), 7.34-7.38 (m, 1H, Ar—H), 7.35 (s, 1H, Ar—H), 7.58 (s, 1H, Ar—H), 7.60 (s, 1H, Ar—H), 8.16 (s, 1H, Ar—H).

¹³C-NMR (CD₃OD, δ): 18.2, 57.5, 57.8, 103.5, 105.7, 107.4, 109.2, 110.3, 120.8, 123.1, 124.0, 127.1, 138.8, 142.9, 150.3, 151.5, 154.3, 155.0, 159.5.

MS-ESI m/z (rel. int.): 324 ([M+H]⁺, 100).

HPLC: Method A, detection UV 254 nm, RT=4.53 min, peak area 99.5%.

### 3-(Benzo[d][1,3]dioxol-5-yl)-6,7-dimethoxy-1,2-dimethylisoquinolinium chloride 22

To a solution of 3-(benzo[d][1,3]dioxol-5-yl)-6,7-dimethoxy-1-methylisoquinoline CCH 18068 (105 mg, 0.32 mmol) in CH₃CN (15 mL) in a 30 mL sealed tube equipped with a magnetic stirrer was added iodomethane (0.40 mL, 6.43 mmol) and the reaction mixture was stirred at 95° C. for 24 h after which it was cooled down and precipitated with Et₂O (15 mL). The solid was filtered and washed several times with Et₂O. Ion exchange on Amberlite IRA-400 (chloride form, 50 eq.) followed by recrystallization from MeOH gave 3-(benzo[d][1,3]dioxol-5-yl)-6,7-dimethoxy-1,2-dimethylisoquinolinium chloride 22 as an off-white solid (94 mg, 62% yield).

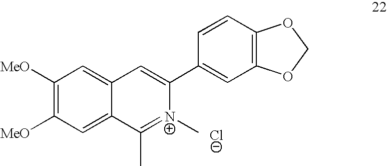

MW: 373.83; Yield: 62%; Off-white solid; Mp (° C.): 217 (dec.).

¹H-NMR (DMSO d₆, δ): 3.21 (s, 3H, CH₃), 4.03 (s, 6H, 2×CH₃), 4.08 (s, 3H, CH₃), 6.19 (s, 2H, OCH₂O), 7.10 (dd, 1H, J=1.7 and 8.0 Hz, Ar—H), 7.21 (d, 1H, J=8.0 Hz, Ar—H), 7.23 (d, 1H, J=1.7 Hz, Ar—H), 7.69 (s, 1H, Ar—H), 7.85 (s, 1H, Ar—H), 8.08 (s, 1H, Ar—H).

¹³C-NMR (CDCl₃:CD₃OD=1:1, δ): 17.2, 42.6, 56.1, 56.4, 102.1, 105.2, 105.7, 108.7, 109.4, 123.3, 123.5, 123.8, 127.1, 135.4, 145.4, 148.5, 149.6, 153.2, 156.1, 157.9.

MS-ESI m/z (rel. int.): 338 ([M]⁺, 100).

HPLC: Method A, detection UV 254 nm, RT=4.46 min, peak area 97.3%.

### 6,7-Dimethoxy-1-methyl-3-(3-nitrophenyl)isoquinolinium chloride 23

To a mixture of 6,7-dimethoxy-1-methylisoquinolin-3-yl trifluoromethanesulfonate CCH 18064 (634 mg, 1.805 mmol) and 3-nitrophenylboronic acid (301 mg, 1.803 mmol) in toluene (22 mL) in a 30 mL sealed tube equipped with a magnetic stirrer was added 2N aqueous Na₂CO₃ (5.4 mL) and the reaction mixture was stirred for 5 min. [1,1′-Bis(diphenylphosphino)ferrocene]dichloropalladium(II) complex (126 mg, 0.154 mmol) was then added and the mixture was stirred overnight at 80° C. After cooling to RT, the aqueous phase was removed and the organic phase was diluted with EtOAc (15 mL) and stirred in presence of charcoal (one spatula) for 10 minutes then filtered through celite, eluting with EtOAc, then with MeOH, then with CH₂Cl₂. The filtrate was dried over MgSO₄ and concentrated under vacuum. The crude product was purified by column chromatography (SiO₂; eluent cyclohexane:EtOAc=5:1 to 3:1, then with CH₂Cl₂) to afford after evaporation to 6,7-dimethoxy-1-methyl-3-(3-nitrophenyl)isoquinoline CCH 18080 as a yellow solid (350 mg, 60% yield).

The solid CCH 18080 was then dissolved in MeOH (11 mL) in a 50 mL round-bottomed flask equipped with a magnetic stirrer and the solution was cooled to 0° C. in an ice bath before adding 3.4 mL of a 0.47 N HCl solution in EtOH. The solution was stirred for 0.4 h at 0° C. before concentration to dryness at RT under vacuum to obtain 6,7-dimethoxy-1-methyl-3-(3-nitrophenyl)isoquinolinium chloride 23 as an off-white solid.

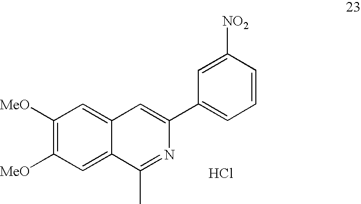

MW: 360.79; Yield: 60% (free base); Off-white solid; Mp (° C.): 217 (dec.).

R_{f}: (free base): 0.3 (cyclohexane:EtOAc=3:1).

¹H-NMR (DMSO d₆, exchange with CD₃OD, δ): 3.22 (s, 3H, CH₃), 4.08 (s, 3H, OCH₃), 4.09 (s, 3H, OCH₃), 7.70 (2s, 2H, Ar—H), 7.93 (dd, 1H, J=8.0 Hz, Ar—H), 8.39-8.45 (m, 2H, Ar—H), 8.48 (s, 1H, Ar—H), 8.86-8.86 (m, 1H, Ar—H).

¹³C-NMR (DMSO d₆, δ): 18.3, 56.5, 56.6, 105.4, 106.8, 120.0, 122.2, 122.9, 124.7, 130.8, 134.4, 134.7, 135.8, 139.2, 148.4, 152.4, 155.3, 156.9.

MS-ESI m/z (rel. int.): 325 ([M+H]⁺, 100).

HPLC: Method A, detection UV 254 nm, RT=4.46 min, peak area 98.5%.

### 1-(3-(6,7-Dimethoxy-1-methylisoquinolin-3-yl)phenyl)ethanone hydrochloride 24

To a mixture of 6,7-dimethoxy-1-methylisoquinolin-3-yl trifluoromethanesulfonate CCH 18064 (423 mg, 1.204 mmol) and 3-acetylphenylboronic acid (197 mg, 1.201 mmol) in toluene (15 mL) in a 30 mL sealed tube equipped with a magnetic stirrer was added 2N aqueous Na₂CO₃ (3.6 mL) and the reaction mixture was stirred for 5 min at RT. [1,1′-Bis(diphenylphosphino)ferrocene]dichloropalladium(II) complex (84 mg, 0.103 mmol) was then added and the mixture was stirred for 4 h at 80° C. After cooling to RT, the aqueous phase was removed and the organic phase was diluted with EtOAc (15 mL), stirred in presence of charcoal (one spatula) for 10 min, filtered through celite (eluting with EtOAc), dried over MgSO₄ and concentrated under vacuum. The crude product was purified by column chromatography (SiO₂; eluent cyclohexane:EtOAc=3:2) to obtain after evaporation to dryness 1-(3-(6,7-dimethoxy-1-methylisoquinolin-3-yl)phenyl)ethanone CCH 18078 as an off-white solid (133 mg, 41% yield).

The solid was then dissolved in MeOH (5 mL) in a 25 mL round-bottomed flask equipped with a magnetic stirrer and the solution was cooled to 0° C. in an ice bath before adding 5.0 mL of a 0.12 N HCl solution in MeOH. The solution was stirred for 0.4 h at 0° C. before concentration to dryness at RT under vacuum to obtain 1-(3-(6,7-dimethoxy-1-methylisoquinolin-3-yl)phenyl)ethanone hydrochloride 24 as an off-white solid.

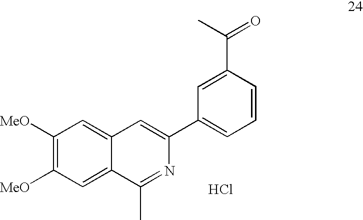

MW: 357.83; Yield: 41% (free base); Off-white solid; Mp (° C.): 182 (dec.).

R_{f}: (free base): 0.25 (cyclohexane:EtOAc=3:2).

¹H-NMR (CD₃OD d₄, δ): 2.75 (s, 3H, CH₃), 3.28 (s, 3H, CH₃), 4.15 (s, 3H, OCH₃), 4.16 (s, 3H, OCH₃), 7.65 (s, 1H, Ar—H), 7.67 (s, 1H, Ar—H), 7.78-7.83 (m, 1H, Ar—H), 8.12 (d, 1H, J=7.4 Hz, Ar—H), 8.24 (d, 1H, J=7.4 Hz, Ar—H), 8.32 (s, 1H, Ar—H), 8.48 (s, 1H, Ar—H).

¹³C-NMR (acetone d₆, δ): 18.9, 26.9, 56.7, 56.9, 105.6, 107.2, 119.4, 123.1, 128.3, 130.0, 130.2, 132.8, 135.6, 136.8, 138.7, 143.3, 153.3, 156.1, 157.6, 197.6.

MS-ESI m/z (rel. int.): 322 ([M+H]⁺, 100).

HPLC: Method A, detection UV 254 nm, RT=4.43 min, peak area 99.3%.

### 3-(3-Acetylphenyl)-6,7-dimethoxy-1,2-dimethylisoquinolinium chloride 25

To a solution of 1-(3-(6,7-dimethoxy-1-methylisoquinolin-3-yl)phenyl)ethanone CCH 18078 (90 mg, 0.28 mmol) in CH₃CN (2 mL) in a 30 mL sealed tube equipped with a magnetic stirrer was added iodomethane (10 mL, 160.63 mmol) and the mixture was stirred for 3 days at 90° C. The reaction mixture was then cooled down to RT and diluted with Et₂O (12 mL). The solid obtained (94 mg) after filtration was dissolved in DMSO (1 mL) and purified by reversed phase HPLC on C18 Xterra Column 19×50 mm, 5 μm part 186001108 with a gradient of 20 to 25% CH₃CN (0.05% TFA) in H₂O (0.05% TFA) in 7 min. After 8 injections, all the selected fractions were combined and evaporated under reduced pressure to give a solid. This solid was immediately dissolved in MeOH (5 mL) at 0° C. in a 25 mL round-bottomed flask equipped with a magnetic stirrer and converted into its chloride salt using 2N aqueous HCl solution (4 mL) to obtain, after concentration to dryness at RT under vacuum, 3-(3-acetylphenyl)-6,7-dimethoxy-1,2-dimethylisoquinolinium chloride 25 as a pale brown solid (67 mg, 64% yield).

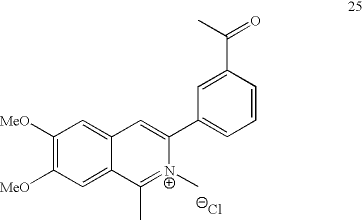

MW: 371.86; Yield: 64%; Pale brown solid; Mp (° C.): 246 (dec.).

¹H-NMR (DMSO d₆, δ): 2.67 (s, 3H, CH₃), 3.26 (s, 3H, CH₃), 4.04 (s, 3H, OCH₃), 4.05 (s, 3H, NCH₃), 4.11 (s, 3H, OCH₃), 7.76 (s, 1H, Ar—H), 7.78-7.86 (m, 1H), 7.89 (s, 1H, Ar—H), 7.89-7.95 (m, 1H, Ar—H), 8.17 (s, 1H, Ar—H), 8.20-8.24 (m, 2H, Ar—H).

¹³C-NMR (DMSO d₆, δ): 18.0, 26.9, 43.4, 56.6, 56.7, 106.1, 106.2, 122.8, 123.0, 129.2, 129.6, 129.7, 133.9, 134.4 (2C), 137.1, 144.0, 152.3, 156.9, 197.4.

MS-ESI m/z (rel. int.): 336 ([M]⁺, 100).

HPLC: Method A, detection UV 254 nm, RT=4.39 min, peak area 97.4%.

### 4-(6,7-Dimethoxy-1-methylisoquinolin-3-yl)benzene-1,2-diol hydrochloride 26

To a suspension of 3-(benzo[d][1,3]dioxol-5-yl)-6,7-dimethoxy-1-methylisoquinoline CCH 18068 (62 mg, 192 μmol) in CH₂Cl₂ (10 mL) at −78° C. in a 50 mL round-bottomed flask equipped with a magnetic stirrer under N₂ was added dropwise BCl₃ (1N solution in CH₂Cl₂, 0.58 mL, 580 μmol) and the reaction mixture was stirred for 1 h at −78° C., then for 3 days at RT. Another portion of BCl₃ (1N solution in CH₂Cl₂, 0.58 mL, 580 μmol) was then added and the mixture was stirred overnight under reflux. After cooling to RT, the mixture was concentrated to dryness under vacuum. The solid was dissolved in MeOH (5 mL) in a 25 mL round-bottomed flask equipped with a magnetic stirrer, 2N aqueous HCl (5 mL) was then carefully added and the mixture was stirred at RT for 40 min, after which the volatiles were removed under vacuum. The solid obtained was dissolved in DMSO (1 mL) and purified using reversed phase HPLC on C18 Xterra Column 19×50 mm, 5 μm part 186001108 with a gradient of 0 to 40% CH₃CN (0.05% TFA) in H₂O (0.05% TFA) in 7 min. After 5 injections, all the selected fractions were combined and evaporated under reduced pressure to give, after concentration of the fractions, ion exchange on Amberlite IRA-400 (chloride form, 50 eq.) and drying under vacuum, 4-(6,7-dimethoxy-1-methylisoquinolin-3-yl)benzene-1,2-diol hydrochloride 26 as a brown solid (8 mg, 13% yield).

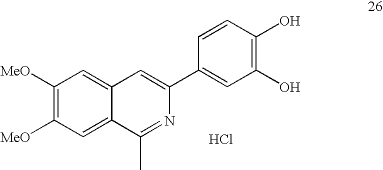

MW: 347.79; Yield: 13%; Brown solid; Mp (° C.): 269.9 (dec.).

¹H-NMR (CD₃OD, δ): 3.17 (s, 3H, CH₃), 4.10 (s, 3H, OCH₃), 4.11 (s, 3H, OCH₃), 7.00 (d, 1H, J=8.2 Hz, Ar—H), 7.22 (dd, 1H, J=1.9 and 8.2 Hz, Ar—H), 7.28 (d, 1H, J=1.9 Hz, Ar—H), 7.58 (s, 1H, Ar—H), 7.62 (s, 1H, Ar—H), 8.12 (s, 1H, Ar—H).

¹³C-NMR (CD₃OD, δ): 18.2, 57.5, 57.8, 105.5, 107.3, 116.0, 117.3, 120.3, 121.3, 122.9, 124.8, 139.0, 143.8, 147.4, 149.6, 154.1, 154.4, 159.5.

MS-ESI m/z (rel. int.): 312 ([M+H]⁺, 100).

HPLC: Method A, detection UV 254 nm, RT=4.12 min, peak area 99.1%.

### 3-(3,4-Dihydroxyphenyl)-6,7-dimethoxy-1,2-dimethylisoquinolinium chloride 27

To a suspension of 3-(benzo[d][1,3]dioxol-5-yl)-6,7-dimethoxy-1,2-dimethylisoquinolinium chloride 22 (57 mg, 152 μmol) in CH₂Cl₂ (10 mL) at −78° C. under N₂ in a 50 mL round-bottomed flask equipped with a magnetic stirrer was added dropwise BCl₃ (1N solution in CH₂Cl₂, 0.46 mL, 460 μmol) and the reaction mixture was stirred for 1 h at −78° C. then for 3 days at RT. Another portion of BCl₃ (1N solution in CH₂Cl₂, 0.46 mL, 460 mmol) was then added and the mixture was stirred overnight at RT then concentrated under vacuum. The solid was dissolved in MeOH (5 mL), 2N aqueous HCl (5 mL) was added and the mixture was stirred at RT for 40 min, after which the volatiles were removed under vacuum. The solid obtained was dissolved in DMSO (1 mL) and purified using reversed phase HPLC on C18 Xterra Column 19×50 mm, 5 μm part 186001108 with a gradient of 0 to 30% CH₃CN (0.05% TFA) in H₂O (0.05% TFA) in 7 min. After 4 injections, all the selected fractions were combined and evaporated under reduced pressure to give a solid. After ion exchange on Amberlite IRA-400 (chloride form, 50 eq.), the desired product was dissolved in MeOH (3 mL) and precipitated from Et₂O. After drying under vacuum, 3-(3,4-dihydroxyphenyl)-6,7-dimethoxy-1,2-dimethylisoquinolinium chloride 27 was obtained as a brown solid (18 mg, 33% yield).

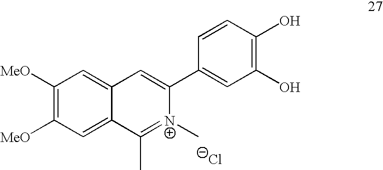

MW: 361.82; Yield: 33%; Brown solid; Mp (° C.): 276 (dec.).

¹H-NMR (CD₃OD, δ): 3.24 (s, 3H, CH₃), 4.11 (s, 3H, CH₃), 4.13 (s, 3H, CH₃), 4.15 (s, 3H, CH₃), 6.91 (d, 1H, J=7.8 Hz), 6.98 (d, 1H, J=7.8 Hz, Ar—H), 7.00 (s, 1H, Ar—H), 7.56 (s, 1H, Ar—H), 7.77 (s, 1H, Ar—H), 7.99 (s, 1H, Ar—H).

¹³C-NMR (CDCl₃:CD₃OD=1:1, δ): 18.8, 44.2, 57.6, 57.9, 106.5, 107.1, 117.0, 117.7, 122.7, 124.5, 124.7, 126.2, 136.9, 147.1, 147.7, 148.8, 154.4, 157.0, 159.2.

MS-ESI m/z (rel. int.): 326 ([M]⁺, 100).

HPLC: Method A, detection UV 254 nm, RT=4.07 min, peak area 99.3%.

### 3-(6,7-Dimethoxy-1-methylisoquinolin-3-yl)aniline dihydrochloride 28

To a solution of 6,7-dimethoxy-1-methyl-3-(3-nitrophenyl)isoquinoline CCH 18080 (242 mg, 746 μmol) in MeOH:CH₂Cl₂:AcOH=20:5:5 (30 mL) in a 250 mL round-bottomed flask equipped with a magnetic stirrer was added Pd/C (10 wt. %, 80 mg) and the reaction mixture was stirred at RT under H₂ (1 atm.) for 2 h, after which the mixture was filtered through celite and concentrated under vacuum. The residual product was partitioned between CH₂Cl₂ (15 mL) and water (10 mL) and the aqueous phase was basified to pH=10 with 6N aqueous NH₄OH solution. The organic phase was isolated and the aqueous phase further extracted with CH₂Cl₂. The combined organic phase was washed with brine, dried over Na₂SO₄, filtered and concentrated under vacuum, to give 3-(6,7-dimethoxy-1-methylisoquinolin-3-yl)aniline CCH 18088 as a pale brown solid (205 mg, 93% yield).

A small portion of the above product CCH 18088 (37 mg) was dissolved in MeOH (5 mL) in a 25 mL round-bottomed flask equipped with a magnetic stirrer and the solution was cooled to 0° C. in an ice bath before adding 3.0 mL of a 0.12 N HCl solution in MeOH. The solution was stirred for 0.4 h at 0° C. before concentration to dryness at RT under vacuum. The desired product was finally recrystallized from MeOH/Et₂O to obtain after drying under vacuum 3-(6,7-dimethoxy-1-methylisoquinolin-3-yl)aniline dihydrochloride 28 as a brown solid (29 mg).

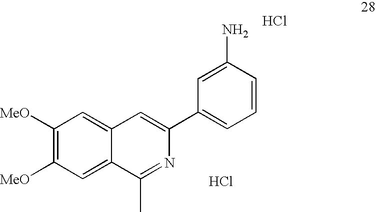

MW: 367.27; Yield: 93% (free base); Brown solid; Mp (° C.): 236 (dec.).

R_{f}: (free base): 0.2 (cyclohexane:EtOAc=1:1).

¹H-NMR (CD₃OD, δ): 3.27 (s, 3H, CH₃), 4.14 (s, 3H, OCH₃), 4.16 (s, 3H, OCH₃), 7.62-7.65 (m, 1H, Ar—H), 7.70 (s, 2H, Ar—H), 7.81 (t, 1H, J=7.9 Hz, Ar—H), 7.96-7.99 (m, 2H, Ar—H), 8.36 (s, 1H, Ar—H).

¹³C-NMR (CDCl₃:CD₃OD=1:1, δ): 18.8, 57.4, 57.7, 106.1, 107.8, 122.0, 123.7, 123.8, 126.0, 129.5, 132.4, 134.0, 135.6, 138.4, 141.0, 154.8, 156.0, 159.8.

MS-ESI m/z (rel. int.): 295 ([M+H]⁺, 100).

HPLC: Method A, detection UV 254 nm, RT=3.79 min, peak area 97.0%.

### N-(3-(6,7-Dimethoxy-1-methylisoquinolin-3-yl)phenyl)-N-(methylsulfonyl)methanesulfonamide hydrochloride 29

To a solution of 3-(6,7-dimethoxy-1-methylisoquinolin-3-yl)aniline CCH 18088 (57 mg, 0.194 mmol) in dry CH₂Cl₂ (5 mL) in a 25 mL round-bottomed flask equipped with a magnetic stirrer under N₂ was added NEt₃ (81 μL, 0.581 mmol) followed by methanesulfonyl chloride (32 μL, 0.41 mmol) and the reaction mixture was stirred overnight at RT. The reaction mixture was then washed with water (3 mL), then with brine (5 mL), dried over Na₂SO₄ and concentrated under vacuum. Purification by column chromatography (SiO₂; eluent cyclohexane:EtOAc=1:1) gave after evaporation and drying under high vacuum N-(3-(6,7-dimethoxy-1-methylisoquinolin-3-yl)phenyl)-N-(methylsulfonyl)methanesulfonamide as a white solid (25 mg, 29% yield).

The solid was dissolved in MeOH (5 mL) in a 25 mL round-bottomed flask equipped with a magnetic stirrer and the solution was cooled to 0° C. in an ice bath before adding 0.7 mL of a 0.12 N HCl solution in MeOH. The solution was stirred for 0.4 h at 0° C. before concentration to dryness at RT under vacuum to obtain N-(3-(6,7-dimethoxy-1-methylisoquinolin-3-yl)phenyl)-N-(methylsulfonyl)methanesulfonamide hydrochloride 29 as a white solid.

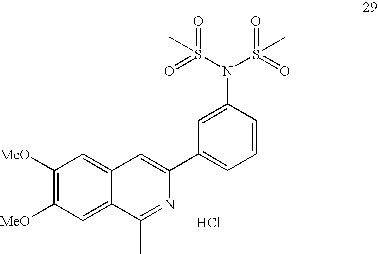

MW: 486.99; Yield: 29% (free base); White solid; Mp (° C.): 244 (dec.).

R_{f}: (free base): 0.2 (cyclohexane:EtOAc=1:1).

¹H-NMR (CD₃OD, δ): 3.27 (s, 3H, CH₃), 3.55 (s, 6H, 2×CH₃), 4.14 (s, 3H, OCH₃), 4.16 (s, 3H, OCH₃), 7.65 (s, 1H, Ar—H), 7.66 (s, 1H, Ar—H), 7.69-7.76 (m, 1H, Ar—H), 7.78 (dd, 1H, J=7.8 Hz, Ar—H), 8.00-8.05 (m, 2H, Ar—H), 8.32 (s, 1H, Ar—H).

¹³C-NMR (CDCl₃:CD₃OD=1:1, δ): 17.9, 43.2 (2×CH₃), 57.1, 57.4, 105.6, 107.4, 121.6, 123.2, 130.8, 131.5, 131.6, 133.9, 134.7, 136.1, 138.0, 140.8, 154.3, 155.2, 159.3.

MS-ESI m/z (rel. int.): 451 ([M+H]⁺, 100).

HPLC: Method A, detection UV 254 nm, RT=4.61 min, peak area 99.5%.

### N-(3-(6,7-Dimethoxy-1-methylisoquinolin-3-yl)phenyl)methanesulfonamide hydrochloride 30

To a solution of 3-(6,7-dimethoxy-1-methylisoquinolin-3-yl)aniline CCH 18088 (57 mg, 0.194 mmol) in dry CH₂Cl₂ (5 mL) in a 25 mL round-bottomed flask equipped with a magnetic stirrer under N₂ was added NEt₃ (81 μL, 0.581 mmol) followed by methanesulfonyl chloride (15 μL, 0.194 mmol) and the reaction mixture was stirred overnight at RT. The reaction mixture was then washed with water (3 mL), then with brine (5 mL), dried over Na₂SO₄ and concentrated under vacuum. Purification by column chromatography (SiO₂; eluent cyclohexane:EtOAc=2:3) gave after evaporation N-(3-(6,7-dimethoxy-1-methylisoquinolin-3-yl)phenyl)methanesulfonamide as a white solid (6 mg, 8% yield).

The solid was dissolved in MeOH (2.5 mL) in a 25 mL round-bottomed flask equipped with a magnetic stirrer and the solution was cooled to 0° C. in an ice bath before adding 0.2 ml, of a 0.12 N HCl solution in MeOH. The solution was stirred for 0.4 h at 0° C. before concentration to dryness at RT under vacuum to obtain N-(3-(6,7-dimethoxy-1-methylisoquinolin-3-yl)phenyl)methanesulfonamide hydrochloride 30 as a white solid.

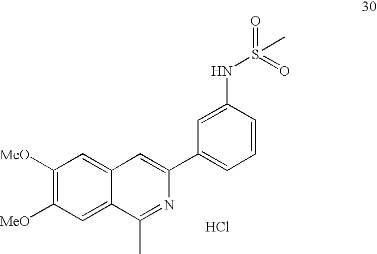

MW: 408.90; Yield: 8% (free base); White solid; Mp (° C.): 233.8 (dec.).

R_{f}: (free base): 0.2 (cyclohexane:EtOAc=2:3).

¹H-NMR (CDCl₃:CD₃OD=1:1, δ): 3.10 (s, 3H, CH₃), 3.27 (s, 3H, CH₃), 4.16 (s, 3H, OCH₃), 4.18 (s, 3H, OCH₃), 7.45-7.49 (m, 1H, Ar—H), 7.60-7.62 (m, 4H, Ar—H), 7.75 (s, 1H, Ar—H), 8.22 (s, 1H, Ar—H).

¹³C-NMR (CDCl₃:CD₃OD=1:1, δ): 18.5, 40.4, 57.7, 58.0, 105.8, 107.7, 120.8, 121.7, 123.5, 125.2, 131.9, 134.6, 138.6, 140.6, 142.5, 154.6, 155.3, 159.7.

MS-ESI m/z (rel. int.): 373 ([M+H]⁺, 100).

HPLC: Method A, detection UV 254 nm, RT=4.31 min, peak area 98.7%.

### N-(3-(6,7-Dimethoxy-1-methylisoquinolin-3-yl)phenyl)acetamide hydrochloride 31

To a solution of 3-(6,7-dimethoxy-1-methylisoquinolin-3-yl)aniline CCH 18088 (53 mg, 180 μmol) in dry CH₂Cl₂ (5 mL) in a 25 mL round-bottomed flask equipped with a magnetic stirrer under N₂ was added NEt₃ (75 μL, 538 μmol) followed by acetyl chloride (30 μL, 422 μmol) and the reaction mixture was stirred overnight at RT. The mixture was washed with water (3 mL), brine (5 mL), dried over Na₂SO₄ and concentrated under vacuum. Purification by column chromatography (SiO₂; eluent EtOAc:cyclohexane=2:1) gave after evaporation N-(3-(6,7-dimethoxy-1-methylisoquinolin-3-yl)phenyl)acetamide as a white solid (43 mg, 71% yield).

The solid was dissolved in MeOH (4 mL) in a 25 mL round-bottomed flask equipped with a magnetic stirrer and the solution was cooled to 0° C. in an ice bath before adding 1.6 mL of a 0.12 N HCl solution in MeOH. The solution was stirred for 0.4 h at 0° C. before concentration to dryness at RT under high vacuum to obtain N-(3-(6,7-dimethoxy-1-methylisoquinolin-3-yl)phenyl)acetamide hydrochloride 31 as a white solid.

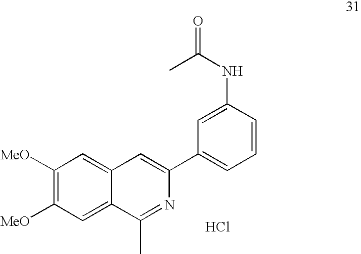

MW: 372.85; Yield: 71% (free base); White solid; Mp (° C.): 238 (dec.).

R_{f}: (free base): 0.2 (cyclohexane:EtOAc=1:2).

¹H-NMR (CDCl₃:CD₃OD=1:1, δ): 2.24 (s, 3H, CH₃), 3.26 (s, 3H, CH₃), 4.16 (s, 3H, OCH₃), 4.18 (s, 3H, OCH₃), 7.55-7.57 (m, 2H, Ar—H), 7.59 (s, 1H, Ar—H), 7.60 (s, 1H, Ar—H), 7.68-7.72 (m, 1H, Ar—H), 8.13-8.14 (m, 1H, Ar—H), 8.22 (s, 1H, Ar—H).

¹³C-NMR (CDCl₃:CD₃OD=1:1, δ): 17.8, 24.0, 57.1, 57.4, 105.1, 107.0, 120.0, 120.8, 122.7, 122.8, 124.1, 130.6, 133.0, 137.9, 140.2, 142.2, 153.8, 154.4, 158.9, 171.5.

MS-ESI m/z (rel. int.): 337 ([M+H]⁺, 100).

HPLC: Method A, detection UV 254 nm, RT=4.23 min, peak area 95.3%.

### Isopropyl 4-(6,7-dimethoxy-1-methylisoquinolin-3-yl)benzoate hydrochloride 32

To a mixture of 6,7-dimethoxy-1-methylisoquinolin-3-yl trifluoromethanesulfonate CCH 18064 (634 mg, 1.805 mmol) and 4-isopropoxycarbonylphenylboronic acid (375 mg, 1.803 mmol) in toluene (20 mL) in a 30 mL sealed tube equipped with a magnetic stirrer was added 2N aqueous Na₂CO₃ (5.4 mL) and the reaction mixture was stirred for 5 min.

[1,1′-Bis(diphenylphosphino)ferrocene]dichloropalladium(II) complex (126 mg, 0.154 mmol) was then added and the mixture was stirred for 4 h at 80° C. After cooling to RT, the organic phase was diluted with EtOAc (15 mL) and the aqueous phase was isolated and further extracted with CH₂Cl₂ (2×10 mL). The organic phase was combined, washed with brine (10 mL), stirred in presence of charcoal (one spatula) and Na₂SO₄, filtered and concentrated under vacuum. The crude product was purified by column chromatography, (SiO₂; eluent CH₂Cl₂:MeOH=160:1) to give after evaporation isopropyl 4-(6,7-dimethoxy-1-methylisoquinolin-3-yl)benzoate CCH 18100 as an off-white solid (320 mg, 49% yield).

The solid was dissolved in MeOH (9 mL) in a 50 mL round-bottomed flask equipped with a magnetic stirrer and the solution was cooled to 0° C. in an ice bath before adding 2.8 mL of a 0.47 N HCl solution in EtOH. The solution was stirred for 0.4 h at 0° C. before concentration to dryness at RT under vacuum to obtain isopropyl 4-(6,7-dimethoxy-1-methylisoquinolin-3-yl)benzoate hydrochloride 32 as a white solid.

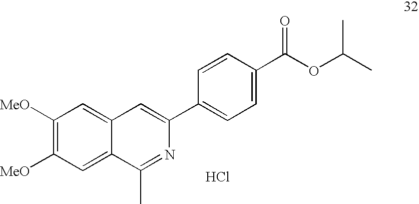

MW: 401.88; Yield: 49% (free base); White Solid; Mp (° C.): 234 (dec.).

R_{f}: (free base): 0.2 (CH₂Cl₂:MeOH=160:1).

¹H-NMR (CD₃OD, δ): 1.42 (d, 6H, J=6.2 Hz, 2×CH₃), 3.23 (s, 3H, CH₃), 4.12 (s, 3H, OCH₃), 4.13 (s, 3H, OCH₃), 5.27 (hept., 1H, J=6.2 Hz, CH(CH₃)₂), 7.66 (s, 1H), 7.70 (s, 1H, Ar—H), 8.01 (d, 2H, J=8.2 Hz, Ar—H), 8.24 (d, 2H, J=8.2 Hz, Ar—H), 8.33 (s, 1H, Ar—H).

¹³C-NMR (CD₃OD, δ): 18.1, 22.1, 57.1, 57.4, 70.5, 106.0, 107.6, 121.5, 123.6, 129.3, 131.3, 133.7, 138.1, 138.2, 141.9, 154.6, 156.1, 159.5, 166.6.

MS-ESI m/z (rel. int.): 366 ([M+H]⁺, 100).

HPLC: Method A, detection UV 254 nm, RT=4.89 min, peak area 97.1%.

### 4-(6,7-Dimethoxy-1-methylisoquinolin-3-yl)benzoic acid hydrochloride 33

To a solution of isopropyl 4-(6,7-dimethoxy-1-methylisoquinolin-3-yl)benzoate CCH 18100 (182 mg, 498 μmol) in MeOH (5 mL) in a 50 mL round-bottomed flask equipped with a magnetic stirrer was added 2N aqueous NaOH (5 mL) and the mixture was stirred under reflux overnight. After cooling to RT, MeOH was evaporated and the reaction mixture was acidified with 13 mL of 1N aqueous HCl solution. The off-white solid that formed was filtrated, washed several times with water and evaporated under vacuum to obtain 4-(6,7-dimethoxy-1-methylisoquinolin-3-yl)benzoic acid hydrochloride 33 as an off-white solid (156 mg, 87% yield).

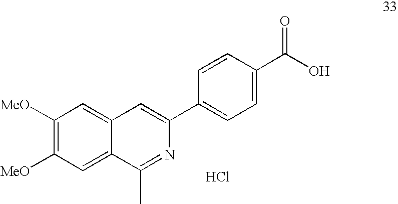

MW: 359.80; Yield: 87%; Off-white solid; Mp (° C.): 265 (dec.).

¹H-NMR (DMSO d₆, δ): 3.21 (s, 3H, CH₃), 4.06 (s, 3H, OCH₃), 4.07 (s, 3H, OCH₃), 7.64 (s, 1H Ar—H), 7.67 (s, 1H Ar—H), 8.10 (d, 2H, J=8.0 Hz Ar—H), 8.15 (d, 2H, J=8.0 Hz Ar—H), 8.38 (s, 1H Ar—H).

MS-ESI m/z (rel. int.): 324 ([M+H]⁺, 100).

HPLC: Method A, detection UV 254 nm, RT=4.12 min, peak area 96.3%.

### 4-(6,7-Dimethoxy-1-methylisoquinolin-3-yl)-N-methylbenzamide 34

To a solution of 4-(6,7-dimethoxy-1-methylisoquinolin-3-yl)benzoic acid hydrochloride 33 (42 mg, 116 μmol) in dry CH₂Cl₂ (5 mL) in a 25 mL round-bottomed flask equipped with a magnetic stirrer under N₂ were added successively oxalyl chloride (1 mL, 11.64 mmol) then a few drops of anhydrous DMF. The reaction mixture was stirred at RT for 2 h, after which it was concentrated under vacuum. The solid was then dissolved in THF (5 mL) at RT, methylamine (40 wt. % in water, 50 μL, 578 μmol) was added and the reaction mixture was stirred overnight at RT. The medium was then diluted with EtOAc (10 mL) and washed with water (5 mL), brine (5 mL), dried over Na₂SO₄ and concentrated under vacuum. After purification by column chromatography (SiO₂; eluent CH₂Cl₂:MeOH=20:1) and evaporation under vacuum 4-(6,7-dimethoxy-1-methylisoquinolin-3-yl)-N-methylbenzamide 34 was isolated as an off-white solid (15 mg, 38%).

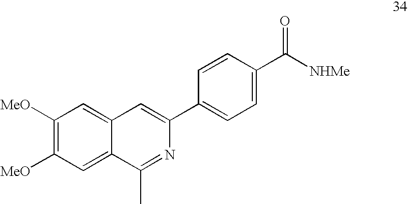

MW: 336.38; Yield: 38%; Off-white solid; Mp (° C.): 248.5.

R_{f}: (free base): 0.2 (CH₂Cl₂:MeOH=20:1).

¹H-NMR (CDCl₃, δ): 2.95 (s, 3H, CH₃), 3.03 (d, 3H, J=4.8 Hz, NHCH₃), 4.03 (s, 3H, OCH₃), 4.05 (s, 3H, OCH₃), 6.36-6.37 (br, m, 1H, NH), 7.10 (s, 1H, Ar—H), 7.26 (s, 1H, Ar—H), 7.82 (s, 1H, Ar—H), 7.86 (d, 2H, J=8.5 Hz, Ar—H), 8.15 (d, 2H, J=8.5 Hz, Ar—H).

¹³C-NMR (CDCl₃, δ): 22.7, 26.8, 56.0, 56.1, 103.9, 105.8, 114.9, 122.6, 126.7, 127.2, 133.3, 133.8, 142.9, 148.8, 150.1, 152.7, 156.1, 168.1.

MS-ESI m/z (rel. int.): 337 ([M+H]⁺, 100).

HPLC: Method A, detection UV 254 nm, RT=3.96 min, peak area 95.7%.

### 6,7-Dimethoxy-3-(6-methoxypyridin-3-yl)-1-methylisoquinoline CCH 18158

To a mixture of 6,7-dimethoxy-1-methylisoquinolin-3-yl trifluoromethanesulfonate CCH 18064 (563 mg, 1.603 mmol) and 2-methoxy-5-pyridineboronic acid (270 mg, 1.765 mmol) in toluene (15 mL) in a 30 mL sealed tube equipped with a magnetic stirrer was added 2N aqueous Na₂CO₃ (3.6 mL) and the reaction mixture was stirred for 5 min at RT. [1,1′-Bis(diphenylphosphino)ferrocene]dichloropalladium(II) complex (112 mg, 137 μmol) was added and the mixture was stirred for 2.5 h at 80° C. After cooling to RT the organic phase was isolated and the aqueous phase was further extracted with CH₂Cl₂ (2×35 mL). The organic phase was combined, washed with brine (20 mL), stirred in presence of charcoal (one spatula) and Na₂SO₄, filtered through celite and concentrated under vacuum. The crude product was finally purified by column chromatography (SiO₂; eluent cyclohexane:EtOAc=2:1) to obtain after evaporation and drying under vacuum 6,7-dimethoxy-3-(6-methoxypyridin-3-yl)-1-methylisoquinoline CCH 18158 as an off-white solid (235 mg, 47% yield).

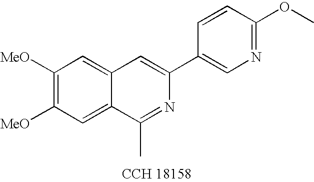

MW: 310.35; Yield: 47%; Off-white solid; Mp (° C.): 131 (dec.).

R_{f}: (free base): 0.25 (cyclohexane:EtOAc=2:1).

¹H-NMR (CDCl₃, δ): 2.93 (s, 3H, CH₃), 4.00 (s, 3H, OCH₃), 4.03 (s, 3H, OCH₃), 4.04 (s, 3H, OCH₃), 6.85 (d, 1H, J=8.6 Hz, Ar—H), 7.08 (s, 1H, Ar—H), 7.25 (s, 1H, Ar—H), 7.70 (s, 1H, Ar—H), 8.33 (dd, 1H, J=2.5 and 8.6 Hz, Ar—H), 8.83 (d, 2H, J=2.5 Hz, Ar—H).

¹³C-NMR (CDCl₃, δ): 22.7, 53.6, 56.0, 56.0, 103.9, 105.5, 110.6, 113.5, 122.1, 129.4, 133.4, 137.3, 145.2, 146.6, 149.8, 152.7, 156.1, 164.0.

MS-ESI m/z (rel. int.): 311 ([M+H]⁺, 100).

HPLC: Method A, detection UV 254 nm, RT=4.08 min, peak area 97.4%.

### 6,7-Dimethoxy-3-(6-methoxypyridin-3-yl)-1-methylisoquinoline dimethanesulfonate 35

6,7-dimethoxy-3-(6-methoxypyridin-3-yl)-1-methylisoquinoline CCH 18158 (36 mg, 116 μmol) was dissolved in CH₂Cl₂ (7 mL) in a 25 mL round-bottomed flask equipped with a magnetic stirrer and the solution was cooled to 0° C. in an ice bath before adding methanesulfonic acid (376 μL, 579 μmol). The solution was stirred for 15 min at 0° C., filtered and the precipitate was washed with Et₂O (2×10 mL), CH₂Cl₂ (10 mL) to give after drying under vacuum 6,7-dimethoxy-3-(6-methoxypyridin-3-yl)-1-methylisoquinoline dimethanesulfonate 35 as a white solid (59 mg, 100% yield).

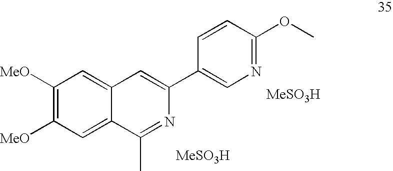

MW: 502.56; Yield: 100%; White solid; Mp (° C.): 238 (dec.).

R_{f}: (free base): 0.25 (cyclohexane:EtOAc=2:1).

¹H-NMR (CD₃OD, δ): 2.70 (s, 6H, 2×CH₃), 3.22 (s, 3H, CH₃), 4.06 (s, 3H, OCH₃), 4.11 (s, 3H, OCH₃), 4.12 (s, 3H, OCH₃), 7.13 (d, 1H, J=8.5 Hz, Ar—H), 7.64 (s, 1H, Ar—H), 7.69 (s, 1H, Ar—H), 8.22-8.30 (m, 2H, Ar—H), 8.70 (s, 1H, Ar—H).

¹³C-NMR (CD₃OD, δ): 18.3, 39.7 (2C), 55.7, 57.5, 57.9, 106.1, 107.7, 112.6, 121.1, 123.2, 123.6, 138.4, 139.6, 141.2, 146.9, 154.1, 156.0, 159.2, 166.5.

MS-ESI m/z (rel. int.): 311 ([M+H]⁺, 100).

HPLC: Method A, detection UV 254 nm, RT=4.19 min, peak area 96.3%.

### 6,7-Dimethoxy-1-methyl-3-(pyridin-3-yl)isoquinoline dihydrochloride 36

To a mixture of 6,7-dimethoxy-1-methylisoquinolin-3-yl trifluoromethanesulfonate CCH 18064 (437 mg, 1.244 mmol) and 3-pyridinylboronic acid (153 mg, 1.245 mmol) in toluene (10 mL) in a 30 mL sealed tube equipped with a magnetic stirrer was added 2N aqueous Na₂CO₃ (2.8 mL) and the reaction mixture was stirred for 5 min at RT. [1,1′-Bis(diphenylphosphino)ferrocene]dichloropalladium(II) complex (88 mg, 108 μmol) was then added and the mixture was stirred for 1 h at 80° C. then for 4 h at 90° C. After cooling to RT, the organic phase was isolated and the aqueous phase was further extracted with EtOAc:THF=1:1 (3×10 mL). The organic phase was combined, washed with brine (10 mL), stirred in presence of charcoal and Na₂SO₄, filtered through celite and concentrated under vacuum. The crude product was finally purified by column chromatography (SiO₂; eluent with EtOAc) to give after evaporation under vacuum 6,7-dimethoxy-1-methyl-3-(pyridin-3-yl)isoquinoline as an off-white solid (32 mg, 9% yield).

The solid was dissolved in MeOH (2 mL) in a 10 mL round-bottomed flask equipped with a magnetic stirrer and the solution was cooled to 0° C. in an ice bath before adding 2.8 mL of a 0.12 N HCl solution in MeOH. The solution was stirred for 0.4 h at 0° C. before concentration to dryness at RT under vacuum to obtain 6,7-dimethoxy-1-methyl-3-(pyridin-3-yl)isoquinoline dihydrochloride 36 as an off-white solid.

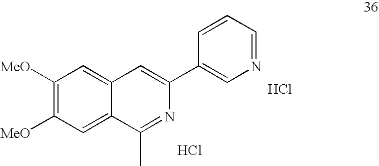

MW: 353.24; Yield: 9% (free base); Off-white solid; Mp (° C.): 230 (dec.).

R_{f}: (free base): 0.3 (EtOAc).

¹H-NMR (CD₃OD, δ): 3.33 (s, 3H, CH₃), 4.15 (s, 3H, OCH₃), 4.16 (s, 3H, OCH₃), 7.78 (s, 1H, Ar—H), 7.79 (s, 1H, Ar—H), 8.39 (dd, 1H, J=5.8 and 8.1 Hz, Ar—H), 8.58 (s, 1H, Ar—H), 9.12 (d, 1H, J=5.8 Hz, Ar—H), 9.22 (dd, 1H, J=1.3 and 8.1 Hz, Ar—H), 9.58 (s, 1H, Ar—H).

¹³C-NMR (CD₃OD, δ): 18.4, 57.4, 57.7, 106.6, 108.3, 123.6, 124.2, 129.0, 133.7, 135.5, 137.7, 143.0, 147.4, 153.3, 157.2, 159.9.

MS-ESI m/z (rel. int.): 281 ([M+H]⁺, 100).

HPLC: Method A, detection UV 254 nm, RT=3.58 min, peak area 99.0%.

### 2-(6,7-Dimethoxy-1-methylisoquinolin-3-yl)aniline dihydrochloride 37

To a mixture of 6,7-dimethoxy-1-methylisoquinolin-3-yl trifluoromethanesulfonate CCH 18064 (713 mg, 2.03 mmol) and 2-aminophenylboronic acid pinacol ester (445 mg, 2.03 mmol) in toluene (25 mL) in a 30 mL sealed tube equipped with a magnetic stirrer was added 2N aqueous Na₂CO₃ (6.1 mL) and the reaction mixture was stirred for 5 min at RT. [1,1′-Bis(diphenylphosphino)ferrocene]dichloropalladium(II) complex (142 mg, 174 μmol) was added and the mixture was stirred overnight at 80° C. The reaction mixture was then cooled to RT and diluted with EtOAc (15 mL) and the aqueous phase was removed. The organic phase was washed with brine (10 mL), filtered through celite, dried over Na₂SO₄, concentrated and purified by column chromatography (SiO₂; eluent cyclohexane:EtOAc=2:1) to give 2-(6,7-dimethoxy-1-methylisoquinolin-3-yl)aniline CCH 18170 as a yellow solid (0.31 g, 52% yield).

The solid CCH 18170 (54 mg, 183 μmol) was dissolved in MeOH (2 mL) in a 10 mL round-bottomed flask equipped with a magnetic stirrer and the solution was cooled to 0° C. in an ice bath before adding a 0.47 N HCl solution in EtOH (12 mL). The solution was stirred for 0.4 h at 0° C. before concentration to dryness at RT under vacuum to obtain 2-(6,7-dimethoxy-1-methylisoquinolin-3-yl)aniline dihydro chloride 37 as a yellow solid.

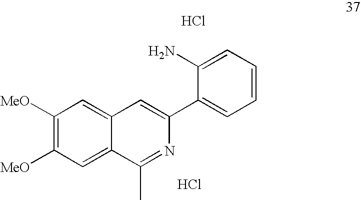

MW: 367.27; Yield: 52% (free base); Yellow solid; Mp (° C.): 244 (dec.).

R_{f}: (free base): 0.3 (cyclohexane:EtOAc=2:1).

¹H-NMR (DMSO d₆, exchange with CD₃OD, δ): 3.19 (s, 3H, CH₃), 4.05 (s, 3H, OCH₃), 4.08 (s, 3H, OCH₃), 7.27-7.56 (m, 4H, Ar—H), 7.65 (s, 1H, Ar—H), 7.74 (s, 1H, Ar—H), 8.34 (s, 1H, Ar—H).

¹³C-NMR (DMSO d₆, δ): 17.6, 56.5, 56.6, 105.4, 106.3, 120.9, 121.7, 121.9, 123.2, 131.3, 131.7, 135.7, 137.9, 151.9, 154.7, 156.6.

MS-ESI m/z (rel. int.): 295 ([M+H]⁺, 100).

HPLC: Method A, detection UV 254 nm, RT=3.96 min, peak area 97.5%.

### N-(2-(6,7-Dimethoxy-1-methylisoquinolin-3-yl)phenyl)acetamide 38

To a solution of 2-(6,7-dimethoxy-1-methylisoquinolin-3-yl)aniline CCH 18170 (48 mg, 163 μmol) in dry CH₂Cl₂ (5 mL) in a 25 mL round-bottomed flask equipped with a magnetic stirrer was added Et₃N (68 μL, 488 μmol) followed by acetyl chloride (27 μL, 378 mmol) and the reaction mixture was stirred for 3 h at RT. The solution was then diluted with CH₂Cl₂ (10 mL), washed with brine (5 mL), dried over Na₂SO₄ and concentrated under vacuum. Purification by column chromatography, (SiO₂; eluent cyclohexane:EtOAc=2:1) afforded after evaporation and drying under vacuum N-(2-(6,7-dimethoxy-1-methylisoquinolin-3-yl)phenyl)acetamide 38 as a white solid (47 mg, 86% yield).

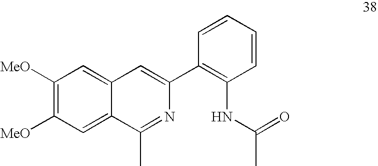

MW: 336.38; Yield: 86%; White solid; Mp (° C.): 246 (dec.).

R_{f}: (free base): 0.2 (cyclohexane:EtOAc=2:1).

¹H-NMR (CDCl₃, δ): 2.20 (s, 3H, CH₃), 2.98 (s, 3H, CH₃), 4.06 (s, 3H, OCH₃), 4.07 (s, 3H, OCH₃), 7.13-7.18 (m, 1H, Ar—H), 7.14 (s, 1H, Ar—H), 7.29 (s, 1H, Ar—H), 7.38 (dd, 1H, J=7.5 Hz, Ar—H), 7.71 (d, 1H, J=7.5 Hz), 7.80 (s, 1H, Ar—H), 8.53 (d, 1H, J=8.2 Hz, Ar—H), 12.58 (br, s, 1H, NH).

¹³C-NMR (CDCl₃, δ): 22.6, 25.2, 56.1, 56.1, 103.6, 105.7, 117.2, 121.6, 121.7, 123.4, 126.2, 128.5, 129.1, 134.0, 137.6, 149.6, 150.4, 153.2, 153.9, 168.3.

MS-ESI m/z (rel. int.): 337 ([M+H]⁺, 94), 359 ([M+Na]⁺, 6).

HPLC: Method A, detection UV 254 nm, RT=3.78 min, peak area 99.3%.

### 3-(3,4-Dichlorophenyl)-6,7-dimethoxy-1-methylisoquinolinylium chloride 39

To a mixture of 6,7-dimethoxy-1-methylisoquinolin-3-yl trifluoromethanesulfonate CCH 18064 (320 mg, 911 μmol) and 3,4-dichlorophenylboronic acid (174 mg, 912 μmol) in toluene (7 mL) in a 30 mL sealed tube equipped with a magnetic stirrer was added 2N aqueous Na₂CO₃ (2.1 mL) and the reaction mixture was stirred for 5 min at RT. [1,1-Bis(diphenylphosphino)ferrocene]dichloropalladium(II) complex (65 mg, 80 μmol) was then added and the mixture was stirred for 1 h at 85° C. After cooling to RT and dilution with EtOAc (15 mL), the organic phase was isolated and the aqueous phase was further extracted with EtOAc:THF=1:1 (3×10 mL). The organic phase was combined, washed with brine (10 mL), stirred in presence of charcoal (one spatula) and Na₂SO₄, filtered and concentrated under vacuum. The crude product was purified by column chromatography (SiO₂; eluent cyclohexane:EtOAc=4:1) to obtain after drying under vacuum 3-(3,4-dichlorophenyl)-6,7-dimethoxy-1-methylisoquinoline as a white solid (132 mg, 42% yield).

The solid was then dissolved in MeOH (6 mL) in a 25 mL round-bottomed flask equipped with a magnetic stirrer and the solution was cooled to 0° C. in an ice bath before adding 1.2 mL of a 0.47 N HCl solution in EtOH. The solution was stirred for 0.4 h at 0° C. before concentration to dryness at RT under vacuum to obtain 3-(3,4-dichlorophenyl)-6,7-dimethoxy-1-methylisoquinolinylium chloride 39 as a white solid.

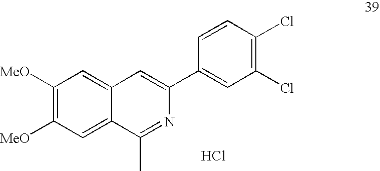

MW: 384.68; Yield: 42% (free base); White solid; Mp (° C.): 234 (dec.).

R_{f}: (free base): 0.3 (cyclohexane:EtOAc=3:1).

¹H-NMR (CDCl₃:CD₃OD=1:1, δ): 3.28 (s, 3H, CH₃), 4.16 (s, 3H, OCH₃), 4.18 (s, 3H, OCH₃), 7.62-7.63 (m, 2H, Ar—H), 7.73 (d, 1H, J=8.3 Hz, Ar—H), 7.81 (d, 1H, J=8.3 Hz, Ar—H), 8.05 (s, 1H, Ar—H), 8.26 (s, 1H, Ar—H).

¹³C-NMR (CDCl₃:CD₃OD=1:1, δ): 17.9, 57.1, 57.4, 105.3, 107.2, 121.1, 122.9, 128.2, 130.5, 132.1, 132.5, 134.2, 135.8, 137.6, 139.7, 154.0, 155.0, 159.0.

MS-ESI m/z (rel. int.): 348-350-352 ([M+H]⁺, 55-38-7).

HPLC: Method A, detection UV 254 nm, RT=4.78 min, peak area 95.5%.

### 6,7-Dimethoxy-3-(4-methoxyphenyl)-1-methylisoquinolinylium chloride 40

To a mixture of 6,7-dimethoxy-1-methylisoquinolin-3-yl trifluoromethanesulfonate CCH 18064 (254 mg, 723 μmol) and 4-methoxyphenylboronic acid (110 mg, 724 μmol) in toluene (7 mL) in a 30 mL sealed tube equipped with a magnetic stirrer was added 2N aqueous Na₂CO₃ (1.7 mL) and the reaction mixture was stirred for 5 minutes at RT. [1,1′-Bis(diphenylphosphino)ferrocene]dichloropalladium(II) complex (52 mg, 64 μmol) was added and the mixture was stirred for 1 h at 85° C. After cooling to RT and dilution with EtOAc (15 mL), the organic phase was isolated and the aqueous phase was further extracted with EtOAc:THF=1:1 (3×10 mL). The organic phase was combined, washed with brine (10 mL), stirred in presence of charcoal (one spatula) and Na₂SO₄, filtered and concentrated under vacuum. The crude product was purified by purification by column chromatography (SiO₂; eluent cyclohexane:EtOAc=3:1) to obtain after drying under vacuum 6,7-dimethoxy-3-(4-methoxyphenyl)-1-methylisoquinoline as an off-white solid (88 mg, 39% yield).

The solid was dissolved in MeOH (3 mL) in a 25 mL round-bottomed flask equipped with a magnetic stirrer and the solution was cooled to 0° C. in an ice bath before adding 3.5 mL of a 0.12 N HCl solution in MeOH. The solution was stirred for 0.4 h at 0° C. before concentration to dryness at RT under vacuum to obtain 6,7-dimethoxy-3-(4-methoxyphenyl)-1-methylisoquinolinylium chloride 40 as an off-white solid.

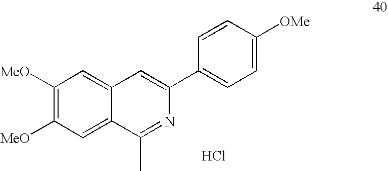

MW: 345.82; Yield: 39% (free base); Off-white solid; Mp (° C.): 219 (dec.)

R_{f}: (free base): 0.25 (cyclohexane:EtOAc=3:1).

¹H-NMR (CDCl₃:CD₃OD=1:1, δ): 3.24 (s, 3H, CH₃), 3.90 (s, 3H, OCH₃), 4.13 (s, 3H, OCH₃), 4.15 (s, 3H, OCH₃), 7.12 (d, 2H, J=8.6 Hz, Ar—H), 7.56 (s, 1H, Ar—H), 7.57 (s, 1H, Ar—H), 7.81 (d, 2H, J=8.6 Hz, Ar—H), 8.15 (s, 1H, Ar—H).

¹³C-NMR (CDCl₃:CD₃OD=1:1, δ): 18.4, 56.5, 57.6, 58.0, 105.7, 107.5, 116.1, 120.5, 123.0, 125.4, 130.7, 138.8, 143.1, 154.2, 154.8, 159.4, 163.2.

MS-ESI m/z (rel. int.): 310 ([M+H]⁺, 100).

HPLC: Method A, detection UV 254 nm, RT=4.31 min, peak area 99.4%.

### 6,7-Dimethoxy-1-methyl-3-(naphthalen-2-yl)isoquinolinylium chloride 41

To a mixture of 6,7-dimethoxy-1-methylisoquinolin-3-yl trifluoromethanesulfonate CCH 18064 (445 mg, 1.267 mmol) and 2-naphthaleneboronic acid (222 mg, 1.291 mmol) in toluene (10 mL) in a 30 mL sealed tube equipped with a magnetic stirrer was added 2N aqueous Na₂CO₃ (2.9 mL) and the reaction mixture was stirred for 5 min at RT. [1,1′-Bis(diphenylphosphino)ferrocene]dichloropalladium(II) complex (90 mg, 110 μmol) was then added and the mixture was stirred for 1 h at 85° C. After cooling to RT and dilution with EtOAc (15 mL), the organic phase was isolated and the aqueous phase was further extracted with EtOAc:THF=1:1 (3×10 mL). The organic phase was combined, washed with brine (10 mL), stirred in presence of charcoal (one spatula) and Na₂SO₄, filtered and concentrated under vacuum. The crude product was finally purified by column chromatography (SiO₂; eluent cyclohexane:EtOAc=3:1) to give 6,7-dimethoxy-1-methyl-3-(naphthalen-2-yl)isoquinoline as a white solid (198 mg, 47% yield).

The solid was then dissolved in MeOH (6 mL) in a 25 mL round-bottomed flask equipped with a magnetic stirrer and the solution was cooled to 0° C. in an ice bath before adding 1.9 mL of a 0.47 N HCl solution in EtOH. The solution was stirred for 0.4 h at 0° C. before concentration to dryness at RT under vacuum to obtain 6,7-dimethoxy-1-methyl-3-(naphthalen-2-yl)isoquinolinylium chloride 41 as an off-white solid. For analytical purpose, a small portion of the sample was recrystallized from MeOH.

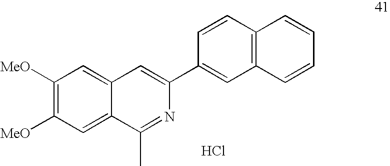

MW: 365.85; Yield: 47% (free base); Off-white solid; Mp (° C.): 242 (dec.).

R_{f}: (free base): 0.4 (cyclohexane:EtOAc=3:1).

¹H-NMR (DMSO d₆ exchange with CD₃OD, δ): 2.89 (s, 3H, CH₃), 3.87 (s, 3H, OCH₃), 3.96 (s, 3H, OCH₃), 6.90 (s, 1H, Ar—H), 7.07 (s, 1H, Ar—H), 7.33-7.41 (m, 3H, Ar—H), 7.50-7.59 (m, 3H, Ar—H), 7.71 (s, 1H, Ar—H), 7.74 (s, 1H, Ar—H).

¹³C-NMR (DMSO d₆, δ): 18.3, 57.1, 57.6, 105.3, 107.1, 120.1, 121.8, 124.4, 127.6, 128.1, 128.3, 128.7, 129.0, 129.2, 129.7, 133.0, 133.8, 136.7, 140.5, 152.3, 154.7, 157.3.

MS-ESI m/z (rel. int.): 330 ([M+H]⁺, 100).

HPLC: Method A, detection UV 254 nm, RT=4.71 min, peak area 99.6%.

### 3-(4-Chlorophenyl)-6,7-dimethoxy-1-methylisoquinolinylium chloride 42

To a mixture of 6,7-dimethoxy-1-methylisoquinolin-3-yl trifluoromethanesulfonate CCH 18064 (349 mg, 993 μmol) and 4-chlorophenylboronic acid (155 mg, 991 μmol) in toluene (10 mL) in a 30 mL sealed tube equipped with a magnetic stirrer was added 2N aqueous Na₂CO₃ (2.4 mL) and the reaction mixture was stirred for 5 min at RT. [1,1′-Bis(diphenylphosphino)ferrocene]dichloropalladium(II) complex (71 mg, 87 μmol) was then added and the mixture was stirred for 1 h at 85° C. After cooling to RT and dilution with EtOAc (15 mL), the organic phase was isolated and the aqueous phase was further extracted with EtOAc:THF=1:1 (3×10 mL). The organic phase was combined, washed with brine (10 mL), stirred in presence of charcoal (one spatula) and Na₂SO₄, filtered and concentrated under vacuum. The crude product was finally purified by column chromatography (SiO₂; eluent cyclohexane:EtOAc=4:1) to give after drying under vacuum 3-(4-Chlorophenyl)-6,7-dimethoxy-1-methylisoquinoline as an off-white solid (171 mg, 55% yield).

The solid was then dissolved in MeOH (6 mL) in a 25 mL round-bottomed flask equipped with a magnetic stirrer and the solution was cooled to 0° C. in an ice bath before adding 1.7 mL of a 0.47 N HCl solution in EtOH. The solution was stirred for 0.4 h at 0° C. before concentration to dryness at RT under vacuum to obtain 3-(4-chlorophenyl)-6,7-dimethoxy-1-methylisoquinolinylium chloride 42 as an off-white solid.

For analytical purpose, a small portion of the sample was recrystallized from MeOH/Et₂O.

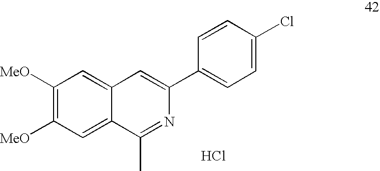

MW: 350.24; Yield: 55% (free base); Off-white solid; Mp (° C.): 236 (dec.).

R_{f}: (free base): 0.33 (cyclohexane:EtOAc=3:1).

¹H-NMR (CDCl₃:CD₃OD=1:1, δ): 3.27 (s, 3H, CH₃), 4.16 (s, 3H, OCH₃), 4.17 (s, 3H, OCH₃), 7.59-7.63 (m, 4H, Ar—H), 7.85 (d, 2H, J=7.9 Hz, Ar—H), 8.22 (s, 1H, Ar—H).

¹³C-NMR (CDCl₃:CD₃OD=1:1, δ): 17.9, 57.0, 57.4, 105.1, 107.0, 120.7, 122.7, 130.1, 130.2, 131.1, 137.8, 141.3, 153.8, 154.7, 158.8.

MS-ESI m/z (rel. int.): 314-316 ([M+H]⁺, 75-25).

HPLC: Method A, detection UV 254 nm, RT=4.68 min, peak area 99.9%.

### 6,7-Dimethoxy-1-methyl-3-p-tolylisoquinolinylium chloride 43

To a mixture of 6,7-dimethoxy-1-methylisoquinolin-3-yl trifluoromethanesulfonate CCH 18064 (318 mg, 905 μmol) and 4-tolylboronic acid (123 mg, 905 μmol) in toluene (9 mL) in a 30 mL sealed tube equipped with a magnetic stirrer was added 2N aqueous Na₂CO₃ (2.2 mL) and the reaction mixture was stirred for 5 min at RT. [1,1′-Bis(diphenylphosphino)ferrocene]dichloropalladium(II) complex (65 mg, 80 μmol) was then added and the mixture was stirred for 1 h at 85° C. After cooling to RT and dilution with EtOAc (15 mL), the organic phase was isolated and the aqueous phase was further extracted with EtOAc:THF=1:1 (3×10 mL). The organic phase was combined, washed with brine (10 mL), stirred in presence of charcoal (one spatula) and Na₂SO₄, filtered and concentrated under vacuum. The crude product was purified by column chromatography (SiO₂; eluent cyclohexane:EtOAc=4:1) to give, after drying under vacuum, 6,7-dimethoxy-1-methyl-3-p-tolylisoquinoline as an off-white solid (139 mg, 52% yield).

The solid was then dissolved in MeOH (5 mL) in a 25 mL round-bottomed flask equipped with a magnetic stirrer and the solution was cooled to 0° C. in an ice bath before adding 1.5 mL of a 0.47 N HCl solution in EtOH. The solution was stirred for 0.4 h at 0° C. before concentration to dryness at RT under vacuum. Finally 7-dimethoxy-1-methyl-3-p-tolylisoquinolinylium chloride 43 was recrystallized from MeOH and obtained, after filtration and drying under high vacuum, as an off-white solid.

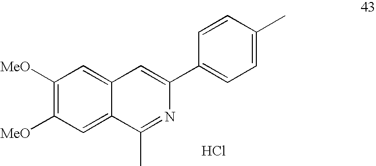

MW: 329.82; Yield: 52% (free base); Off-white solid; Mp (° C.): 222 (dec.).

R_{f}: (free base): 0.35 (cyclohexane:EtOAc=3:1).

¹H-NMR (DMSO d₆ exchange with CD₃OD, δ): 2.41 (s, 3H, CH₃), 3.26 (s, 3H, CH₃), 3.75-4.00 (br, s, 1H, NH), 4.04 (s, 3H, OCH₃), 4.05 (s, 3H, OCH₃), 7.41 (d, 2H, J=8.1 Hz, Ar—H), 7.64 (s, 1H, Ar—H), 7.68 (s, 1H, Ar—H), 7.88 (d, 2H, J=8.1 Hz, Ar—H), 8.31 (s, 1H, Ar—H).

¹³C-NMR (DMSO d₆, δ): 17.5, 20.8, 56.3, 56.5, 105.1, 106.3, 118.7, 121.3, 128.0, 129.2, 129.4, 135.8, 140.1, 140.9, 151.6, 154.7, 156.6.

MS-ESI m/z (rel. int.): 294 ([M+H]⁺, 100).

HPLC: Method A, detection UV 254 nm, RT=4.63 min, peak area 99.8%.

### 6,7-Dimethoxy-1-methyl-3-phenylisoquinolinylium chloride 44

To a mixture of 6,7-dimethoxy-1-methylisoquinolin-3-yl trifluoromethanesulfonate CCH 18064 (318 mg, 905 μmol) and phenylboronic acid (124 mg, 1.017 mmol) in toluene (9 mL) in a 30 mL sealed tube equipped with a magnetic stirrer was added 2N aqueous Na₂CO₃ (2.2 mL) and the reaction mixture was stirred for 5 min. [1,1′-Bis(diphenylphosphino)ferrocene]dichloropalladium(II) complex (71 mg, 87 μmol) was then added and the mixture was stirred at 85° C. for 1 h. After cooling to RT and dilution with EtOAc (15 mL), the organic phase was isolated and the aqueous phase was further extracted with EtOAc:THF=1:1 (3×10 mL). The organic phase was combined, washed with brine (10 mL), stirred in presence of charcoal (one spatula) and Na₂SO₄, filtered and concentrated under vacuum. The crude product was purified by column chromatography (SiO₂; eluent cyclohexane:EtOAc=4:1) to give after drying under vacuum 6,7-dimethoxy-1-methyl-3-phenylisoquinolinylium as a pale yellow solid (237 mg, 94% yield).

The solid was then dissolved in MeOH (8 mL) in a 25 mL round-bottomed flask equipped with a magnetic stirrer and the solution was cooled to 0° C. in an ice bath before adding 2.7 ml, of a 0.47 N HCl solution in EtOH. The solution was stirred for 0.4 h at 0° C. before concentration to dryness at RT under vacuum. Finally 6,7-dimethoxy-1-methyl-3-phenylisoquinolinylium chloride 44 was recrystallized from MeOH and obtained, after filtration and drying under high vacuum, as a pale yellow solid (124 mg, 43% yield).

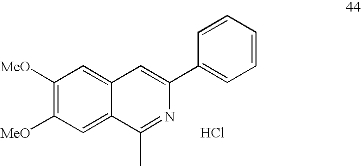

MW: 315.79; Yield: 43%; Pale yellow solid; Mp (° C.): 215 (dec.).

R_{f}: (free base): 0.3 (cyclohexane:EtOAc=3:1).

¹H-NMR (DMSO d₆, exchange with CD₃OD d₄, δ): 3.28 (s, 3H, CH₃), 4.07 (2s, 6H, 2×OCH₃), 7.60-7.64 (m, 3H, Ar—H), 7.67 (s, 1H, Ar—H), 7.71 (s, 1H, Ar—H), 7.96-7.99 (m, 2H, Ar—H), 8.35 (s, 1H, Ar—H).

¹³C-NMR (DMSO d₆, δ): 17.7, 56.4, 56.5, 105.1, 106.4, 119.4, 121.6, 128.2, 129.0, 130.2, 132.3, 136.0, 141.0, 152.0, 154.6, 156.8.

MS-ESI m/z (rel. int.): 280 ([M+H]⁺, 100).

HPLC: Method A, detection UV 254 nm, RT=4.37 min, peak area 99.6%.

### 3-(3,4-Dihydroxyphenyl)-6,7-dihydroxy-1,2-dimethylisoquinolinium chloride 45

3-(Benzo[d][1,3]dioxol-5-yl)-6,7-dimethoxy-1-methylisoquinoline CCH 18068 (198 mg, 612 μmol) was dissolved in THF (5 mL) in a 30 mL sealed tube equipped with a magnetic stirrer and iodomethane (1.0 mL, 16.1 mmol) was added. The reaction mixture was stirred at 85° C. for 5 days after which it was cooled down to RT and filtered. The solid was washed several times with THF (5×10 mL) and dried under vacuum, which gave 152 mg of a pale yellow solid. The solid (152 mg, 327 μmol) was then suspended in dry CH₂Cl₂ (10 mL) in a 50 mL round-bottomed flask equipped with a magnetic stirrer and the medium was cooled to −78° C. before dropwise addition of BBr₃ (1N solution in CH₂Cl₂, 2.0 mL, 2.0 mmol). After complete addition, the reaction mixture was allowed to warm up to RT and stirred under reflux for 3 days, during which two additional portions of BBr₃ were added (2.0 mL and 5.0 mL respectively). The medium was then cooled down to RT, quenched with a mixture of MeOH:6N aqueous HCl solution=1:1 (15 mL) and stirred overnight under reflux. After cooling to RT, the mixture was concentrated under vacuum and purified by preparative HPLC. Evaporation of the fractions containing the desired product followed by ion exchange on Amberlite IRA-400 (chloride form, 50 eq.) gave 3-(3,4-dihydroxyphenyl)-6,7-dihydroxy-1,2-dimethylisoquinolinium chloride 45 as a pale yellow solid (24 mg, 12% yield).

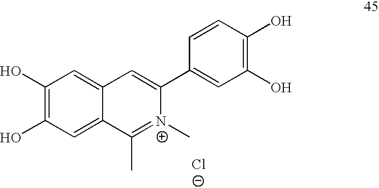

MW: 333.77; Yield: 12%; Pale yellow solid; Mp (° C.): 270 (dec.).

¹H-NMR (CD₃OD, δ): 3.10 (s, 3H, CH₃), 4.07 (s, 3H, CH₃), 6.88 (dd, 1H, J=1.7 and 8.1 Hz, Ar—H), 6.94-6.97 (m, 2H, Ar—H), 7.26 (s, 1H, Ar—H), 7.67 (s, 1H, Ar—H), 7.75 (s, 1H, Ar—H).

¹³C-NMR (CD₃OD, δ): 18.0, 43.4, 110.0, 116.8, 117.7, 122.7, 123.8, 124.0, 126.8, 136.1, 146.4, 147.0, 148.6, 152.0, 156.5, 157.6.

MS-ESI m/z (rel. int.): 298 ([M]⁺, 100).

HPLC: Method A, detection UV 254 nm, RT=3.68 min, peak area 99.5%.

### 3-(2-(6,7-Dimethoxy-1-methylisoquinolin-3-yl)phenyl)-1,1-dimethylurea hydrochloride 46

To a solution of 2-(6,7-dimethoxy-1-methylisoquinolin-3-yl)aniline CCH 18170 (88 mg, 299 μmol) in dry CH₂Cl₂ (5 mL) in a 25 mL round-bottomed flask equipped with a magnetic stirrer under N₂ was added Et₃N (83 μL, 595 μmol) followed by dimethylcarbamoyl chloride (33 μL, 358 μmol) and the reaction mixture was stirred overnight at RT. Another portion of dimethylcarbamoyl chloride (33 μL, 358 μmol) was then added and stirring was continued under reflux for 4 h, then another portion of dimethylcarbamoyl chloride (33 μL, 358 μmol) was added and the mixture was stirred at RT for 48 h. The solution was then diluted with CH₂Cl₂ (10 mL), washed with brine (5 mL), dried over Na₂SO₄ and concentrated under vacuum. Purification by column chromatography (SiO₂; eluent cyclohexane:EtOAc=2:3) gave after drying under vacuum 3-(2-(6,7-dimethoxy-1-methylisoquinolin-3-yl)phenyl)-1,1-dimethylurea as a pale yellow solid (48 mg 44%).

The solid was then dissolved in MeOH (2 mL) in a 10 mL round-bottomed flask equipped with a magnetic stirrer and the solution was cooled to 0° C. in an ice bath before adding 1.6 ml, of a 0.12 N HCl solution in MeOH. The solution was stirred for 0.4 h at 0° C. before concentration to dryness at RT under vacuum to obtain 3-(2-(6,7-dimethoxy-1-methylisoquinolin-3-yl)phenyl)-1,1-dimethylurea hydrochloride 46 as a yellow solid.

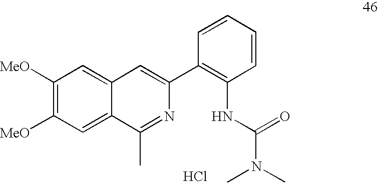

MW: 401.89; Yield: 44% (free base); Yellow solid; Mp (° C.): 182 (dec.).

R_{f}: (free base): 0.22 (cyclohexane:EtOAc=2:3).

¹H-NMR (CD₃OD, δ): 2.86 (s, 6H, 2×CH₃), 3.17 (s, 3H, CH₃), 4.08 (s, 3H, OCH₃), 4.09 (s, 3H, OCH₃), 7.40-7.45 (m, 2H, Ar—H), 7.54-7.64 (m, 4H, Ar—H), 8.07 (s, 1H, Ar—H).

¹³C-NMR (CD₃OD, δ): 17.9, 36.9, 57.2, 57.5, 105.9, 107.3, 122.2, 122.9, 127.3, 128.3, 130.7, 132.0, 132.3, 138.2, 138.6, 141.7, 154.2, 154.7, 159.1, 159.2.

MS-ESI m/z (rel. int.): 366 ([M+H]⁺, 100).

HPLC: Method A, detection UV 254 nm, RT=3.98 min, peak area 99.6%.

### 4-(6,7-Dimethoxy-1-methylisoquinolin-3-yl)-2-methoxyphenol hydrochloride 47

To a mixture of 6,7-dimethoxy-1-methylisoquinolin-3-yl trifluoromethanesulfonate CCH 18064 (318 mg, 905 μmol) and 2-methoxy-4-(4,4,5,5-tetramethyl-1,3,2-dioxaborolan-2-yl)phenol (219 mg, 876 μmol) in toluene (9 mL) in a 30 mL sealed tube equipped with a magnetic stirrer was added 2N aqueous Na₂CO₃ (2.2 mL) and the reaction mixture was stirred for 5 min at RT.

[1,1′-Bis(diphenylphosphino)ferrocene]dichloropalladium(II) complex (65 mg, 80 mmol) was added and the mixture was stirred for 1 h at 85° C. After cooling to RT and dilution with EtOAc (15 mL), the organic phase was isolated and the aqueous phase was further extracted with EtOAc:THF=1:1 (3×10 mL). The organic phase was combined, washed with brine (10 mL), stirred in presence of charcoal (one spatula) and Na₂SO₄, filtered and concentrated under vacuum. The crude product was purified by column chromatography (SiO₂; eluent cyclohexane:EtOAc=3:2) to give after drying under vacuum 4-(6,7-dimethoxy-1-methylisoquinolin-3-yl)-2-methoxyphenol as a yellow solid (45 mg, 16% yield).

The solid was then dissolved in MeOH (2 mL) in a 25 mL round-bottomed flask equipped with a magnetic stirrer and the solution was cooled to 0° C. in an ice bath before adding 3.4 ml, of a 0.12 N HCl solution in MeOH. The solution was stirred for 0.4 h at 0° C. before concentration to dryness at RT under vacuum to obtain 4-(6,7-dimethoxy-1-methylisoquinolin-3-yl)-2-methoxyphenol hydrochloride 47 as a yellow solid.

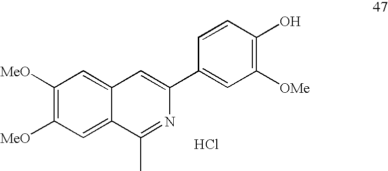

MW: 361.82; Yield: 16% (free base); Yellow solid; Mp (° C.): 264 (dec.).

R_{f}: (free base): 0.35 (cyclohexane:EtOAc=1:1).

¹H-NMR (DMSO d₆, exchange with CD₃OD, δ): 3.21 (s, 3H, CH₃), 3.96 (s, 3H, OCH₃), 4.07 (s, 6H, 2×OCH₃), 7.03-7.05 (m, 1H, Ar—H), 7.37-7.40 (m, 1H, Ar—H), 7.52 (s, 1H, Ar—H), 7.64 (s, 2H, Ar—H), 8.28 (s, 1H, Ar—H).

¹³C-NMR (DMSO, δ): 17.7, 56.1, 56.5, 56.7, 105.3, 106.3, 112.3, 116.1, 118.6, 121.3, 121.5, 123.4, 136.7, 141.5, 148.3, 149.2, 152.1, 154.3, 157.3.

MS-ESI m/z (rel. int.): 326 ([M+H]⁺, 100).

HPLC: Method A, detection UV 254 nm, RT=4.22 min, peak area 99.8%.

### 4-(6,7-Dimethoxy-1-methylisoquinolin-3-yl)benzene-1,2-diol hydrochloride 48

To a solution of 3-(benzo[d][1,3]dioxol-5-yl)-6,7-dimethoxy-1-methylisoquinoline CCH 18068 (207 mg, 0.640 mmol) in dry CH₂Cl₂ (10 mL) in a 100 mL round-bottomed flask equipped with a magnetic stirrer at −78° C. under N₂ was added dropwise BBr₃ (1 N solution in CH₂Cl₂, 4.75 mL, 4.75 mmol). After complete addition, the bath was immediately removed and stirring was continued overnight at RT. The reaction mixture was then carefully quenched with MeOH (20 mL) then with 6N aqueous HCl (5 mL) and stirred at 55° C. for 1 h, after which it was concentrated under vacuum. The crude product was purified by reversed phase column chromatography (LiChroprep® RP-18 (25-40 μm) 11 g; eluent from H₂O:CH₃CN:TFA=100:1:1 to 100:20:1) which gave, after concentration and ion exchange on Amberlite IRA-400 (chloride form, 50 eq.), 4-(6,7-dimethoxy-1-methylisoquinolin-3-yl)benzene-1,2-diol hydrochloride 48 as a yellow solid (42 mg, 21% yield).

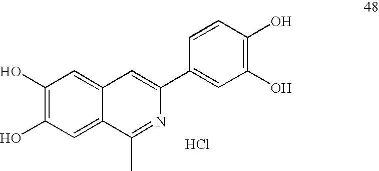

MW: 319.74; Yield: 21%; Yellow solid; Mp (° C.): 219 (dec.).

¹H-NMR (CD₃OD, δ): 3.05 (s, 3H, CH₃), 6.95 (d, 1H, J=8.0 Hz, Ar—H), 7.16 (d, 1H, J=8.0 Hz, Ar—H), 7.22 (s, 1H, Ar—H), 7.30 (s, 1H, Ar—H), 7.53 (s, 1H, Ar—H), 7.86 (s, 1H, Ar—H).

¹³C-NMR (CD₃OD, δ): 17.6, 109.2, 110.3, 115.7, 117.0, 119.2, 121.0, 122.4, 125.1, 138.0, 142.1, 147.3, 149.2, 151.6, 154.0, 157.8.

MS-ESI m/z (rel. int.): 284 ([M+H]⁺, 100).

HPLC: Method A, detection UV 254 nm, RT=3.68 min, peak area 98.6%.

### 6,7-Dimethoxy-3-phenylisoquinoline EBE 10168

To a solution of phenylisocyanide (0.518 mL, 3.44 mmol) in dry THF (20 mL) at −78° C. was added a solution of 1.6 M butylithium in hexanes (2.15 mL, 3.44 mmol) with continuous stirring for 20 min and a solution of 3,4-dimethoxybenzaldehyde (0.572 g, 3.44 mmol) in THF (10 mL) at −78° C. was transferred via a cannula. The reaction mixture was stirred for another hour at −78° C., MeOH (10 mL) and the mixture was allowed to warm to room temperature. All the volatiles were evaporated and the mixture was partitioned between EtOAc (100 mL) and a solution of NaHSO₃ (25 g NaHSO₃ in 50 mL water). The aqueous phase was discarded and the organic layer was washed with brine to give after evaporation a residue that was purified by column chromatography (SiO₂; eluent EtOAc) to give after evaporation crude (±)-trans-5-(3,4-dimethoxyphenyl)-4-phenyl-4,5-dihydrooxazole EBE 10166 (267 mg, 27% yield) as a pale yellow oil.

To a solution of POCl₃ (0.440 g) in CH₃CN (10 mL) was added crude (±)-trans-5-(3,4-dimethoxyphenyl)-4-phenyl-4,5-dihydrooxazole EBE 10166 (267 mg). The reaction mixture was heated at 85° C. for 2 h. The volatiles were evaporated under reduced pressure to obtain an oily residue that was treated with 6 N HCl (5 mL). The aqueous solution was poured in a separatory funnel, washed with CH₂Cl₂ (4×20 mL) and treated at 0° C. with 2N NaOH (20 mL) and the desired product was extracted with CH₂Cl₂ (4×20 mL) to give a residue that was purified by column chromatography (SiO₂; using a gradient of 0-30% EtOAc in cyclohexane) to obtain after drying under vacuum 6,7-dimethoxy-3-phenylisoquinoline EBE 10168 as a pale yellow oil (99 mg, 11% yield from phenylisocyanide).

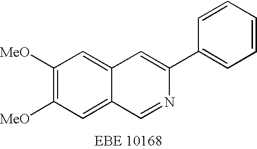

MW: 265.31; Yield: 11%; Pale yellow oil.

R_{f}:: 0.3 (EtOAc).

¹H-NMR (CDCl₃, δ): 4.05 (s, 6H, OMe), 7.12 (s, 1H, Ar—H), 7.22 (s, 1H, Ar—H), 7.36-7.42 (m, 1H, Ar—H), 7.50 (t, 2H, J=6.0 Hz, Ar—H), 7.94 (s, 1H, Ar—H), 8.85 (d, 2H, J=9.0 Hz, Ar—H), 9.13 (s, 1H, Ar—H).

¹³C-NMR (CDCl₃, δ): 56.15 (2×C), 105.0, 105.3, 115.5, 126.8, 127.7 (2×C), 128.2, 128.7 (2×C), 133.4, 139.9, 149.8, 150.3, 153.2.

MS-ESI m/z (rel. int.): 266.0 ([M+H]⁺, 100).

HPLC: Method A, detection UV 254 nm, RT=4.20 min, peak area 98.0%.

### 6,7-Dimethoxy-3-phenylisoquinolinium chloride 49

To a solution of 6,7-dimethoxy-3-phenylisoquinoline EBE 10168 (25 mg, 0.094 mmol) in MeOH (1 mL) at 4° C. was added a solution of 0.13 M HCl in MeOH (2.17 mL, 0.283 mmol). The solution was stirred for 10 min and the volatiles were evaporated to give a solid that was dried over P₂O₅ under high vacuum to give 6,7-dimethoxy-3-phenylisoquinolinium chloride 49, as pale yellow solid (28 mg, 100% yield).

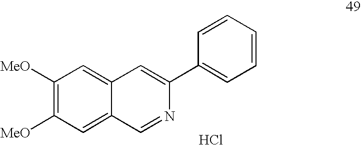

MW: 301.77; Yield: 100%; Pale yellow solid; Mp (° C.): 219.2.

¹H-NMR (CD₃OD, δ): 4.07 (s, 3H, OMe), 4.13 (s, 3H, OMe), 7.63-7.70 (m, 4H, Ar—H), 7.78 (s, 1H, Ar—H), 7.90-7.96 (m, 2H, Ar—H), 8.48 (s, 1H, Ar—H), 9.36 (s, 1H, Ar—H).

¹³C-NMR (CD₃OD, δ): 57.1, 57.6, 106.9, 107.8, 122.0, 124.5, 128.6 (2×C), 130.8 (2×C), 132.1, 133.3, 139.5, 143.0, 143.6, 154.7, 160.2.

MS-ESI m/z (rel. int.): 266.0 ([M+H]⁺, 100).

HPLC: Method A, detection UV 254 nm, RT=4.50 min, peak area 98.0%.

### 6,7-Dimethoxy-2-methyl-3-phenylisoquinolinium chloride 50

A solution of 6,7-dimethoxy-3-phenylisoquinolinium chloride 49 (30 mg) in MeI (1 mL) was prepared and stirred at reflux for 16 h. The iodomethane was evaporated under reduced pressure and the resulting residue was dissolved in a 1:1 solution of water:acetone that was poured on a column (1×8 cm) of Amberlite IR-A 410 resin (Cl⁻ form, 10 eq.). The column was washed with a mixture acetone:water=1:1 (10 mL) and all the 254 nm UV positive fractions were collected, evaporated to dryness and further dried over P₂O₅ under high vacuum to give 6,7-dimethoxy-2-methyl-3-phenylisoquinolinium chloride 50 as pale yellow solid (22 mg, 62% yield).

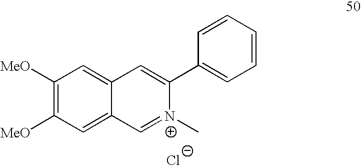

MW: 315.79; Yield: 62%; Pale yellow solid; Mp (° C.): 156.4.

¹H-NMR (CD₃OD, δ): 4.09 (s, 3H, OMe), 4.12 (s, 3H, OMe), 4.19 (s, 3H, N⁺Me), 7.74 (s, 1H, Ar—H), 8.15 (s, 1H, Ar—H), 9.50 (s, 1H, Ar—H).

¹³C-NMR (CD₃OD, δ): 47.3, 57.2, 57.6, 106.4, 107.5, 125.3, 125.9, 130.3 (2×C), 130.8 (2×C), 131.8, 134.0, 138.0, 146.4, 147.9, 155.0, 160.5.

MS-ESI m/z (rel. int.): 280.1 ([M+H]⁺, 100).

HPLC: Method A, detection UV 254 nm, RT=4.51 min, peak area >99.0%.

### Preparation of Compounds 51 and 52

### 2,3-Dihydroxy-7,8-dimethoxy-5-methylbenzo[c]phenanthridinium chloride 51

To a solution of chelerythrine mixture 14 (ratio about 50:50 of chelerythrine and its reduced form) (89 mg, 232 μmol approx.) in boiling ethanol (22 mL) in a 100 mL round-bottomed flask equipped with a magnetic stirrer and a condenser were added sodium acetate (445 mg, 5.425 mmol) followed by iodine (122 mg, 481 μmol) and the reaction mixture was stirred under reflux for 2 h, after which it was concentrated under vacuum. The residue was taken up in CH₂Cl₂:MeOH=7:1 (40 mL), washed with water (8 mL) then with 1N aqueous sodium bisulfite solution (8 mL) then with brine (8 mL), dried over Na₂SO₄ and concentrated under vacuum, which gave crude chelerythrine iodide as a brown solid (63 mg).

To a solution of the above solid (63 mg) in dry CH₂Cl₂ (10 mL) at 0° C. in a 100 mL round-bottomed flask equipped with a magnetic stirrer under N₂ was added dropwise BCl₃ (1N solution in CH₂Cl₂, 1.6 mL, 1.6 mmol) and the reaction mixture was stirred for 15 min at RT then under reflux overnight. The medium was then cooled down to RT, carefully quenched with MeOH (20 mL) and diluted with 6N aqueous HCl (1 mL). This mixture was stirred at RT for 4 h, after which it was concentrated under vacuum. Purification by preparative HPLC followed by ion exchange on Amberlite IRA-400 (chloride form) afforded 2,3-dihydroxy-7,8-dimethoxy-5-methylbenzo[c]phenanthridinium chloride 51 as an orange solid (21 mg, 24% overall yield).

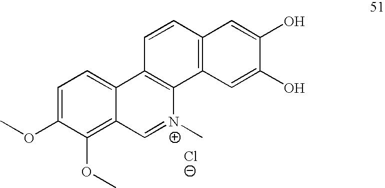

MW: 371.81; Yield: 24%; Orange solid; Mp (° C.): 215 (dec.).

¹H-NMR (DMSO d₆, exchange with CD₃OD, δ): 4.11 (s, 3H, CH₃), 4.17 (s, 3H, CH₃), 4.99 (s, 3H, CH₃), 7.55 (s, 1H, Ar—H), 8.17 (d, 1H, J=8.8 Hz, Ar—H), 8.24 (d, 1H, J=8.8 Hz, Ar—H), 8.45 (s, 1H, Ar—H), 8.65 (d, 1H, J=8.8 Hz, Ar—H), 8.80 (d, 1H, J=8.8 Hz, Ar—H), 10.00 (s, 1H, Ar—H).

¹³C-NMR (DMSO d₆, δ): 52.5, 57.0, 62.1, 111.0, 122.2, 117.0, 118.2, 119.0, 119.2, 124.5, 125.9, 128.3, 130.2, 130.6, 131.0, 145.0, 147.7, 148.3, 149.6, 150.1.

MS-ESI m/z (rel. int.): 336 ([M]⁺, 100).

HPLC: Method A, detection UV 254 nm, RT=4.04 min, peak area 98.7%.

### 2,3,7,8-Tetrahydroxy-5-methylbenzo[c]phenanthridinium chloride 52

To a solution of chelerythrine mixture 14 (ratio about 50:50 of chelerythrine and its reduced form) (138 mg, 359 μmol approx.) in boiling ethanol (30 mL) in a 100 mL round-bottomed flask equipped with a magnetic stirrer and a condenser were added sodium acetate (660 mg, 8.05 mmol) followed by iodine (181 mg, 713 μmol) and the reaction mixture was stirred under reflux for 2 h, after which it was concentrated under vacuum. The residue was taken up in CH₂Cl₂:MeOH=7:1 (64 mL), washed with water (10 mL) then with 1N aqueous sodium bisulfite solution (10 mL) then with brine (10 mL), dried over Na₂SO₄ and concentrated under vacuum, which gave crude chelerythrine iodide as a brown solid (45 mg).

To a solution of the above solid (45 mg) in dry CH₂Cl₂ (15 mL) at −78° C. in a 100 mL round-bottomed flask equipped with a magnetic stirrer under N₂ was added dropwise BBr₃ (1N solution in CH₂Cl₂, 1.2 mL, 1.2 mmol). After complete addition, the reaction mixture was allowed to warm up to RT and stirred overnight. The medium was then carefully quenched with MeOH (20 mL) and diluted with 6N aqueous HCl (1 mL). This mixture was stirred at RT for 4 h, after which it was concentrated under vacuum. The solid obtained was dissolved in DMSO (1 mL) and purified using reversed phase HPLC on C18 Xterra Column 19×50 mm, 5 μm part 186001108 with a gradient of 12 to 17% CH₃CN (0.05% TFA) in H₂O (0.05% TFA) in 7 min. After 5 injections, all the selected fractions were combined and evaporated under reduced pressure to give a solid. After ion exchange on Amberlite IRA-400 (chloride form), concentration to dryness and repeated washing with EtOAc afforded 2,3,7,8-tetrahydroxy-5-methylbenzo[c]phenanthridinium chloride 52 as a dark orange solid (37 mg, 30% overall yield).

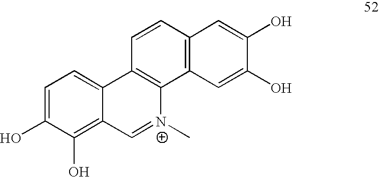

MW: 343.76; Yield: 30%; Dark orange solid; Mp (° C.): 175.2.

¹H-NMR (DMSO d₆, δ): 4.92 (s, 3H, CH₃), 7.52 (s, 1H, Ar—H), 7.94 (d, 1H, J=8.7 Hz, Ar—H), 8.12 (d, 1H, J=8.7 Hz, Ar—H), 8.34 (s, 1H, Ar—H), 8.36 (d, 1H, J=8.7 Hz, Ar—H), 8.53 (d, 1H, J=8.7 Hz, Ar—H), 9.95 (s, 1H, Ar—H), 10.39 (br, s, 1H, OH), 10.50 (br, s, 1H, OH), 10.88 (br, s, 1H, OH), 11.18 (br, s, 1H, OH).

¹³C-NMR (DMSO d₆, δ): 51.9, 111.0, 112.2, 113.9, 115.8, 117.0, 118.4, 124.6, 127.2, 128.5, 130.0, 130.1, 130.3, 143.4, 143.9, 147.5, 148.1, 149.4.

MS-ESI m/z (rel. int.): 308 ([M]⁺, 100).

HPLC: Method A, detection UV 254 nm, RT=3.63 min, peak area 98.7%.

### Material and Methods for Biological Assays

### Preparation of Recombinant Proteins:

The pGEX-Rac1, pGEX-Rac1b, pGEX-Cdc42, pGEX-RhoA and pPRO-Tiam1 DH/PH constructs were kindly provided by C.J. Der (University of North Carolina). BL21 Codon+RIL E. coli strain carrying the constructs of interest is grown in LB medium in presence of 100 μg/ml ampicillin at 37° C. 180 rpm during 3 h until optical density reaches 0.6. The expression of recombinant protein is induced by 1 mM IPTG and the culture continued for further 16 h at 20° C.

After centrifugation at 4000 g, 20 min 4° C., the bacterial pellet is resuspended in 40 ml/l of culture lysis buffer (50 mM Tris pH 8.6, 300 mM NaCl, 1 mM DTT, 1 μg/ml leupeptine, 1 μg/ml pepstatine, 1 mM benzonase, 2 mM MgCl₂, 50 μg/ml lysozyme and 1 mM EDTA). The solution is incubated 30 min. at 4° C. after addition of lysozyme and bacterial lysis is performed by sonication on ice/ethanol. Addition of 1 μM bezonase in lysate is followed by an incubation for 1 h at 4° C. with agitation. A centrifugation at 46000 g, 30 min at 4° C. allows separating soluble from insoluble proteins.

GST-protein was purified on GST-sepharose 6 Fast Flow (Amersham Bioscience). His6-Tiam1-DH/PH was purified using NI-sepharose 6 Fast Flow (Amersham Bioscience). Pooled fraction from the elution was dialysed against a buffer containing 20 mM Tris pH 8.6, 50 mM NaCl, 1 mM MgCl₂. Aliquots are stored at −80° C. in the presence of 10% glycerol. Protein purifications were done by Protein' eXpert (Grenoble).

### Nucleotide Binding Assay:

Fluorescence of GTP analog BODIPY-GTP increases when it binds to small G proteins. This property was used to assess ability of compounds to inhibit nucleotide binding to Rac1, Rac1b and Cdc42. 2 μM GST-Rac1 and 6 μg His6-Tiam1-DH/PH were diluted in a buffer containing 20 mM Tris, 50 mM NaCl, and 1 mM MgCl₂. This mixture was loaded in a 96-well plate. Fluorescence recording were started (λ_{Ex}: 485 nm; λ_{Em}: 538 nm) using a spectrofluorimeter (Fluoroskan; Thermolab Systems). Assay was initiated by the addition of 2 μM BODIPY-GTP with or without various doses of test compound. Fluorescence values were recorded, and data were processed as follows: background fluorescence (unbound BODIPY-GTP) was retrieved from fluorescence values. Curves were then analyzed using the GraphPad Prism software which allows performing non-linear regression (One phase exponential association equation: Y=Ymax.(1−^{-k.X})) Results were expressed as % inhibition=100×(Ymax “compound”/Ymax “no compound”).

### Results of Biological Assays

Ability of protoberberine derivatives and benzo[c]phenanthridine alkaloids to affect small G proteins activity was studied using a biochemical exchange assay. This assay allows determining whether compounds can inhibit the binding of a fluorescent nucleotide to recombinant small G proteins of interest. The effect of various alkaloid compounds on binding of BODIPY-GTP to Rac1 activated by DH/PH domain of Guanine nucleotide Exchange Factor Tiam1 (Rac1/Tiam1), Rac1b and Cdc42 is described below. Activity of the compounds is compared to that of NSC 23766a compound known for its ability to interfere with Rac1 activation by Tiam1.

### Results for Protoberberine Derivatives:

In total, 20 compounds have been tested at 50 μM (see Table 1).
The compounds showing the highest inhibitory activity on binding of BODIPY-GTP to Rac1/Tiam are 2,3,9,10-tetrahydroxyberberine chloride, compound 6 (100%), followed by (±)-tetrahydroxytetrahydroberberine hydrochloride, compound 16 (70±6% inhibition) and 8-methyl-isoquino[3,2-a]isoquinolinylium-2,3,10,11-tetraol chloride, compound 15 (63±4%).
The compounds showing the highest inhibitory activity on Rac1b are compounds 6 (100%), 15 (72±4%) and 16 (67±9%).
The compounds showing the highest inhibitory activity on Cdc42 are compounds 6 (100%), 15 (70±3%) and demethyleneberberine 4 (31±9%).

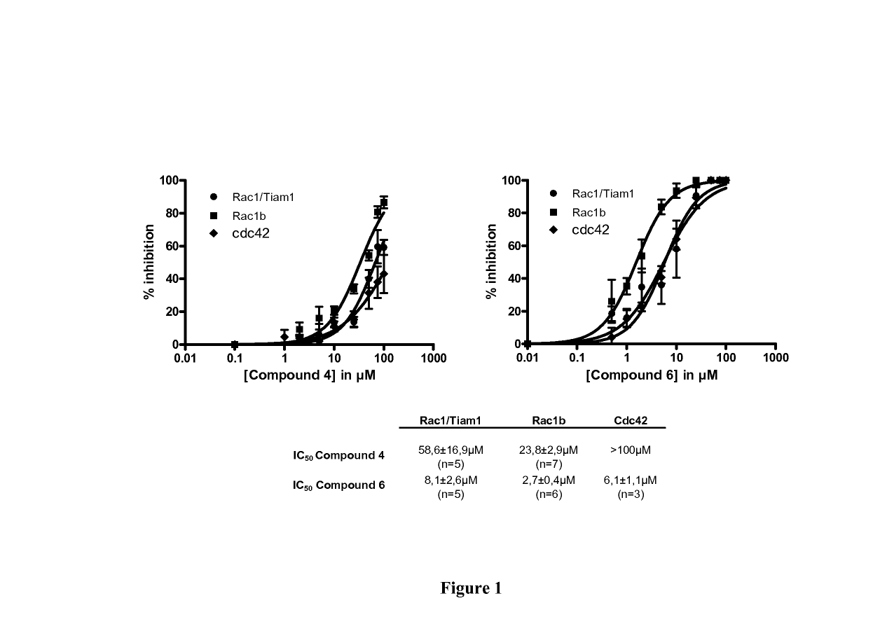

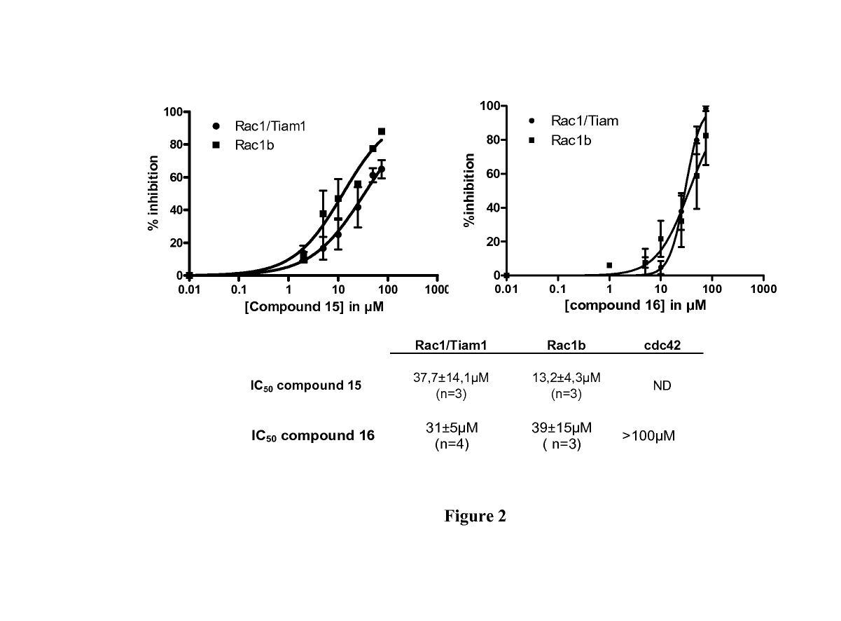

In order to refine these results, dose-response studies were performed on compounds berberine 1, palmatine 2, 4, 6, 15 and 16 (1-2-5-10-25-50-75-100 μM). For compound 1, maximum inhibition was of 19±1% on Rac1/Tiam1 and of 29±1% on Rac1b. For compound 2, maximum inhibitions were of 17±1% and 24±2%, for Rac1/Tiam1 and Rac1b, respectively. IC₅₀s could thus not be determined for these compounds. For compounds 4 and 6, IC₅₀s were calculated and are shown on FIG. 1. Highest activity compound is compound 6, with IC₅₀s of 2.7±0.4 μM for Rac1b and of 8.1±2.6 μM for Rac1/Tiam1. Compound 4 has IC₅₀s of 23.8±2.9 μM and 58.6±16.9 μM for Rac1b and Rac1/Tiam1 respectively. These compounds are about two-fold more active on Rac1b than on Rac1/Tiam1. FIG. 2 shows dose-response curves obtained for compounds 15 and 16. Compound 15 is 3-fold more active on Rac1b (IC₅₀ of 13.2±4.3 μM) than on Rac1/Tiam1. (±)-Tetrahydroxytetrahydroberberine hydrochloride, compound 16 is equipotent between Rac1/Tiam1 (IC₅₀ of 31±5 μM) and Rac1b (IC₅₀ of 39±15 μM) but has no inhibition on Cdc42.

### Results for 3-Aryl-Isoquinolines and Analogs (Ring-Opened Analogs Lacking C5-C6 Bond of Coralyne):

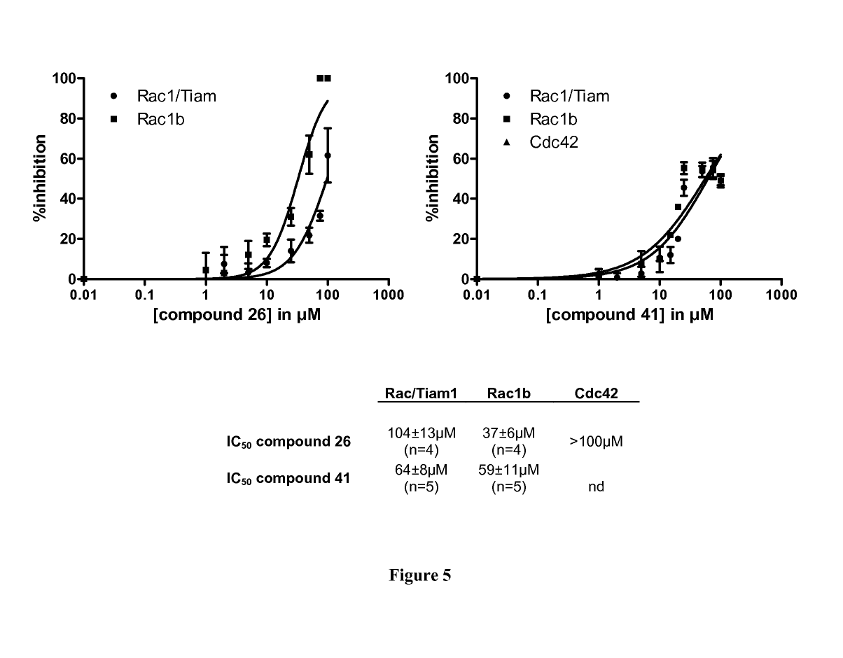

In total, 29 compounds have been tested at 50 μM (see Table 2). Most of the compounds tested seems to demonstrate less inhibition on Rac, Rac1b protein than the protoberberine and benzo[c]phenanthridine alkaloids. However the three best compounds of this series are compounds 41>26>39. IC₅₀s were calculated when possible (see FIG. 5).

Inhibition of compound 41 at 50 μM is similar for Rac1 (48±2%), Rac1b (52±4%) and (31±3%). IC₅₀s were calculated for Rac1 (64±8 μM) and Rac1b (59±11 μM).

Inhibition of compound 26 at 50 μM is for Rac1 (25±3%), Rac1b (46±5%) and Cdc42 (15±1%). The compound 26 showed some selectivity: IC₅₀s for Rac1b (37±6 μM) was about three time better than for Rac1 (104±13 μM).

Results for Benzo[c]phenanthridine Alkaloids:

In total, four compounds have been tested at 50 μM (see Table 3).

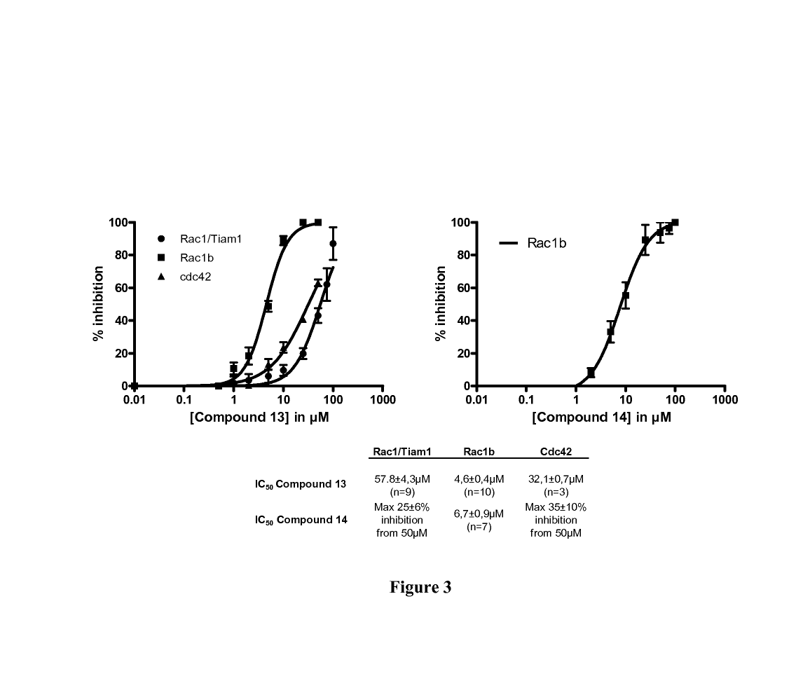

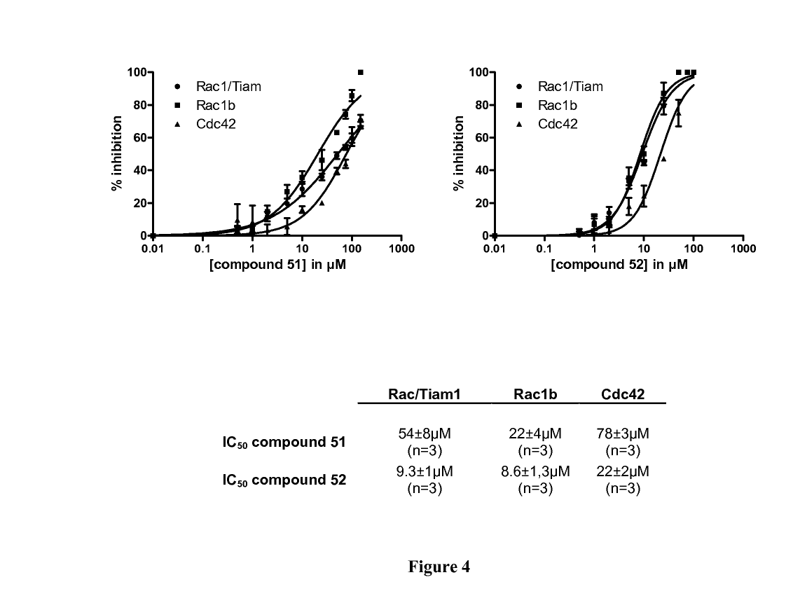

In order to check for compounds selectivity, dose-response studies were performed and IC₅₀s were calculated when possible (see FIGS. 3 and 4).

Chelerythrine 14 is not able to fully inhibit Rac1/Tiam with a max inhibition of around 25% being observed from 50 μM. A similar effect is observed on Cdc42 (35% max inhibition from 50 μM). IC₅₀ for Rac1b is of 6.7±0.9 μM. Chelerythrine 14 is thus highly selective for Rac1b. Chelerythrine 14 display the best selectivity on Rac1b.

Sanguinarine 13 has IC₅₀s of 4.6±0.4 μM for Rac1b, 57.8±4.3 μM for Rac1/Tiam1 and 32.1±0.7 μM for Cdc42. It is thus 13 times and 6 times more active on Rac1b than on Rac1/Tiam1 and Cdc42 respectively.

Compound 51 has IC₅₀s of 22±4 μM for Rac1b, 54±8 μM for Rac1/Tiam1 and 78±3 μM for Cdc42.

Compound 52 has IC₅₀s of 8.6±1.3 μM for Rac1b, 9.3±1 μM for Rac1/Tiam1 and 22±2 μM for Cdc42.

As opposed to protoberberine derivatives, benzo[c]phenanthridine alkaloids sanguinarine 13, compound 51 and chelerythrine 14 showed some significant selectivity for Rac1b. Compound 52 did not show any selectivity for Rac1b versus Rac1 or Cdc42.

Selective compounds would thus be usable for different kinds of pathologies requiring specific or non-specific inhibition of Rac family members.

### Results for Control Compound NSC 23766:

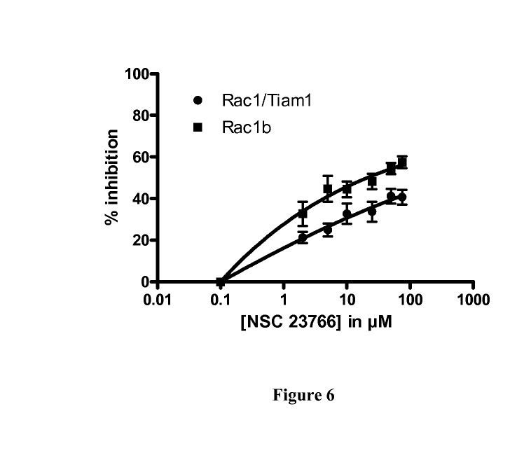

As shown in FIG. 6, NSC 23766 dose-dependently affects binding of BODIPY-GTP to Rac1/Tiam1 and Rac1b. At maximal dose, NSC 23766 inhibits Rac1/Tiam1 of 33±5% and Rac1b of 57±5%.

### Rac1b Activation Assay (G-Lisa):

Rac1b activation assay: HEK293 cells overexpressing human Rac1b were plated in 100 mm diameter dishes and grown for 5 days in MEM medium supplemented with 10% FBS, L-Glutamine and antibiotics. Cells were then treated with 50 μM of compounds or the solvent for 5 minutes, and the GTP loading of Rac1b was measured using a Rac Activation Assay Kit (Cytoskeleton) according to manufacturer's recommendations. Briefly, this assay uses a 96-well plate coated with RBD domain of Rho-family effector proteins. The active GTP-bound form of the Rho-family protein, but not the inactive GDP-bound form, from the biological sample binds to the plate. Bound active Rac protein is detected by incubation with a specific primary antibody followed by a secondary antibody conjugated to HRP. The signal is then developed with OPD reagent.

G-LISA Assay on compounds

| | G-LISA |
|---|---|
| Compounds | % inhibition @ 50 μM |
| 4 | 30 ± 5 |
| 13 | 50 ± 3 |
| 14 | 40 ± 10 |
| 24 | 19 ± 5 |
| 26 | 61 ± 6 |
| 28 | 27 ± 4 |
| 30 | 12 ± 2 |
| 47 | 13 ± 8 |
| 51 | 53 ± 8 |
| NSC 23766 | 7 ± 2 |
| EHT 1864 | 47 ± 2 |

The compounds in the above table demonstrated significant better inhibition of Rac1b than NSC 23766. Best compounds in this assay were 3-arylisoquinoline 26>benzo[c]phenantridine 51>sanguinarine 13>EHT 1864>chelerythrine 14.

### Soft Agar Assay:

Colony formation in soft agar is the most widely used assay to evaluate anchorage-independent growth potential and represents one of the best in vitro assays that correlates strongly with in vivo tumorigenic cell growth potential. Normal cells require adherence and spreading onto a solid substratum in order to remain viable and proliferate. In contrast, cancer cell line HCT116 (ATCC clone number CCL-247) lost this requirement and therefore can form proliferating colonies of cells when suspended in a semisolid agar medium. The assay was performed in 24-well plates, using duplicates wells for each compound concentration. Briefly, a 0.5% Bacto™ Agar (BD Biosciences) bottom layer (prepared in HCT116 complete growth medium supplemented or not with the tested compound) was poured first and allowed to harden (0.3 ml per well). HCT116 were trypsinized to generate a uniform single cell suspension. Five×10³ cells per well were resuspended in 0.3% agar supplemented with complete growth medium, with or without several compound concentrations to form the top layer (0.3 ml per well). HCT116 were allowed to grow for 7 days in these conditions and then analysed for colonies formation. Analysis was performed from pictures taken under a microscope that are representative from 2 different fields of the well. For each field, 2 different focal plans were taken and were merged using ImageJ software. The number of colonies were scored and colonies' size were measured. IC₅₀ were determined for the number and size of colonies using GraphPad Prism (GraphPad) software. Data are mean values of 2 to 3 independent experiments.

Soft agar assay: IC₅₀ (μM) of compounds (HCT116 cell line)

| | Clone Number | Clone Size |
|---|---|---|
| 16 | 6.4 ± 1.3 | 6.2 ± 0.4 |
| 51 | >50 | 11.4 ± 1.9 |
| 26 | 14.8 ± 3.3 | 8.5 ± 0.9 |
| 41 | 70.7 ± 5.9 | 44.4 ± 4.5 |
| 52 | 9.8 ± 1.3 | 9.5 ± 0.4 |
| 14 | 2.4 ± 0.2 | 2.0 ± 0.3 |
| 13 | 1.9 ± 0.6 | 1.5 ± 0.2 |
| NSC 23766 | 64.2 ± 4.8 | 27.9 ± 5.0 |
| EHT 1864 | 57.5 ± 4.5 | 19.5 ± 2.5 |

From the tested compounds, sanguinarine 13 and chelerythrine 14 are the two more potent compounds in this assay, followed by (±)-tetrahydroxytetrahydroberberine hydrochloride 16, 2,3,7,8-tetrahydroxy-5-methyl benzo[c]phenanthridinium chloride 52 and 4-(6,7-dimethoxy-1-methylisoquinolin-3-yl)benzene-1,2-diol hydrochloride 26. Compound 51 has a particular interesting profile having an IC₅₀ (clone number) >50 μM but an IC₅₀ (clone size) of 11.4±1.9 μM. The compounds showing low micromolar IC₅₀ in a soft agar assay (HCT116 cancer cell line) have among the best inhibition of Rac1 and Rac1b.

In conclusion, alkaloid compounds are significantly more active on small G proteins Rac than NSC 23766, a compound extensively described for its Rac1 inhibitory activity (Akbar H., Cancelas J., Williams D. A., Zheng J., and Zheng Y., Methods Enzymol., 2006, 406, 554-65; Gao Y., Dickerson J. B., Guo F., Zheng J., and Zheng Y., Proc Natl Acad Sci USA., 2004, 101, 7618-23).

TABLE 1
Protoberberine Derivatives (Isoquinoline Alkaloids): Formula (I)

| # | Name | R_{I}¹ | R_{I}² | R_{I}³ | R_{I}⁹ | R_{I}¹⁰ | R_{I}¹¹ | A | B |
|---|---|---|---|---|---|---|---|---|---|
| 1 | Berberine chloride | H | OCH₂O | | OMe | OMe | H | N⁺ | CH |
| 2 | Palmatine chloride, hydrate | H | OMe | OMe | OMe | OMe | H | N⁺ | CH |
| 3 | (±)-Canadine hydrochloride | H | OCH₂O | | OMe | OMe | H | N | CH₂ |
| 4 | Demethyleneberberine chloride | H | OH | OH | OMe | OMe | H | N⁺ | CH |
| 5 | (±)-N-benzyl canadinium bromide | H | OCH₂O | | OMe | OMe | H | N⁺Bnz | CH₂ |
| 6 | 2,3,9,10-Tetrahydroxyberberine | H | OH | OH | OH | OH | H | N⁺ | CH |
| | chloride | | | | | | | | |
| 7 | 2-(2,3-Dimethoxybenzyl)-6,7- | H | OMe | OMe | OMe | OMe | H | N | CH₂ |
| | dimethoxy-1,2,3,4- | | | | | | | | |
| | tetrahydroisoquinoline HCl | | | | | | | | |
| 8 | Coralyne chloride, hydrate | H | OMe | OMe | H | OMe | OMe | N⁺ | CMe |
| 9 | Papaverine hydrochloride | H | OMe | OMe | OMe | OMe | H | N | — |
| 10 | 9,10-Dimethoxy-8-phenyl-5,8-dihydro- | H | OCH₂O | | OMe | OMe | H | N | CPh |
| | 2H-6H-[1,3]dioxolo[4,5-g]isoquino[3,2- | | | | | | | | |
| | a]isoquinoline | | | | | | | | |
| 11 | 8-Benzyl-9,10-dimethoxy-5,8-dihydro- | H | OCH₂O | | OMe | OMe | H | N | CBnz |
| | 2H-6H-[1,3]dioxolo[4,5-g]isoquino[3,2- | | | | | | | | |
| | a]isoquinoline | | | | | | | | |
| 12 | (±)-8-Benzyl-9,10-dimethoxy- | H | OCH₂O | | OMe | OMe | H | N | CBnz |
| | 5,8,13,13a-tetrahydro-6H- | | | | | | | | |
| | [1,3]dioxolo[4,5-g]isoquino[3,2- | | | | | | | | |
| | a]isoquinoline HCl | | | | | | | | |
| 15 | 8-Methyl-isoquino[3,2- | H | OH | OH | H | OH | OH | N⁺ | CMe |
| | a]isoquinolinylium-2,3,10,11-tetraol | | | | | | | | |
| | chloride | | | | | | | | |
| 16 | (±)-Tetrahydroxytetrahydroberberine | H | OH | OH | OH | OH | H | N | CH₂ |
| | hydrochloride | | | | | | | | |
| 17 | (±)-9,10-Dimethoxy-5,8,13,13a- | H | OH | OH | OMe | OMe | H | N | CH₂ |
| | tetrahydro-6H-isoquino[3,2- | | | | | | | | |
| | a]isoquinoline-2,3-diol HCl | | | | | | | | |
| 18 | 3-((6,7-Dimethoxy-3,4- | H | OMe | OMe | OH | OH | H | N | CH₂ |
| | dihydroisoquinolin-2(1H)- | | | | | | | | |
| | yl)methyl)benzene-1,2-diol HCl | | | | | | | | |
| 19 | 2-(2,3-Dihydroxybenzyl)-1,2,3,4- | H | OH | OH | OH | OH | H | N | CH₂ |
| | tetrahydroisoquinoline-6,7-diol HCl | | | | | | | | |

| | | | | | | | | |
|---|---|---|---|---|---|---|---|---|
| 20 | (±)-3-(6-ethylbenzo[d][1,3]dioxol-5-yl)- | CH₂CH₃ | OCH₂O | OMe | OMe | H | NCH₃ | CH₂ |
| | 7,8-dimethoxy-2-methyl-1,2,3,4- | | | | | | | |
| | tetrahydroisoquinoline hydrochloride | | | | | | | |

| # | D | E | F | G | J | Rac1 | Rac1b | Cdc42 |
|---|---|---|---|---|---|---|---|---|
| 1 | CH | C | CH₂ | CH₂ | C | 15 ± 2 | 23 ± 2 | 8 ± 2 |
| 2 | CH | C | CH₂ | CH₂ | C | 15 ± 2 | 19 ± 3 | 11 ± 2 |
| 3 | CH₂ | CH | CH₂ | CH₂ | C | 1 ± 3 | −7 ± 4 | ND |
| 4 | CH | C | CH₂ | CH₂ | C | 39 ± 2 | 54 ± 2 | 31 ± 9 |
| 5 | CH₂ | CH | CH₂ | CH₂ | C | 9 ± 2 | 16 ± 2 | 3 ± 1 |
| 6 | CH | C | CH₂ | CH₂ | C | 100 | 100 | 100 |
| 7 | — | CH₂ | CH₂ | CH₂ | C | 0 ± 3 | 6 ± 3 | −1 ± 1 |
| 8 | CH | C | CH | CH | C | 36 ± 4 | 44 ± 4 | 21 ± 2 |
| 9 | CH₂ | C | CH | CH | C | 1 ± 2 | 5 ± 2 | 2 ± 2 |
| 10 | CH | C | CH₂ | CH₂ | C | 18 ± 2 | 27 ± 2 | 17 ± 0 |
| 11 | CH | C | CH₂ | CH₂ | C | −1 ± 2 | −2 ± 3 | 1 ± 1 |
| 12 | CH₂ | CH | CH₂ | CH₂ | C | 23 ± 3 | 31 ± 4 | 9 ± 2 |
| 15 | CH | C | CH | CH | C | 63 ± 4 | 72 ± 4 | 70 ± 3 |
| 16 | CH₂ | CH | CH₂ | CH₂ | C | 70 ± 6 | 67 ± 9 | 4 ± 6 |
| 17 | CH₂ | CH | CH₂ | CH₂ | C | 5 ± 3 | −7 ± 4 | −13 ± 12 |
| 18 | — | CH₂ | CH₂ | CH₂ | C | 1 ± 2 | 4 ± 3 | −8 ± 10 |
| 19 | — | CH₂ | CH₂ | CH₂ | C | 9 ± 2 | 19 ± 3 | 17 ± 3 |
| 20 | CH₂ | CH₂ | — | — | C | 4 ± 4 | −6 ± 10 | 8 ± 3 |

ND: not determined
All compounds were tested at a final concentration of 50 μM

TABLE 2
3-Aryl-isoquinolines (ring-opened analogs lacking G-F bond of coralyne) Formula (V)

| # | Name | R_{I}¹ | R_{I}² | R_{I}³ | R_{I}⁹ | R_{I}¹⁰ | R_{I}¹¹ | A | B |
|---|---|---|---|---|---|---|---|---|---|
| 50 | 6,7-Dimethoxy-2-methyl-3- | H | H | H | H | OMe | OMe | N⁺CH₃ | CH |
| | phenylisoquinolinium chloride | | | | | | | | |
| 49 | 6,7-Dimethoxy-3-phenylisoquinoline | H | H | H | H | OMe | OMe | N | CH |
| | hydrochloride | | | | | | | | |
| 21 | 3-(Benzo[d][1,3]dioxol-5-yl)-6,7- | H | OCH₂O | | H | OMe | OMe | N | CCH₃ |
| | dimethoxy-1-methylisoquinoline HCl | | | | | | | | |
| 22 | 3-(Benzo[d][1,3]dioxol-5-yl)-6,7- | H | OCH₂O | | H | OMe | OMe | N⁺CH₃ | CCH₃ |
| | dimethoxy-1,2- | | | | | | | | |
| | dimethylisoquinolinium chloride | | | | | | | | |
| 23 | 6,7-Dimethoxy-1-methyl-3-(3- | H | NO₂ | H | H | OMe | OMe | N | CCH₃ |
| | nitrophenyl)isoquinoline | | | | | | | | |
| | hydrochloride | | | | | | | | |
| 26 | 4-(6,7-Dimethoxy-1- | H | OH | OH | H | OMe | OMe | N | CCH₃ |
| | methylisoquinolin-3-yl)benzene-1,2- | | | | | | | | |
| | diol hydrochloride | | | | | | | | |
| 27 | 3-(3,4-Dihydroxyphenyl)-6,7- | H | OH | OH | H | OMe | OMe | N⁺CH₃ | CCH₃ |
| | dimethoxy-1,2- | | | | | | | | |
| | dimethylisoquinolinium chloride | | | | | | | | |
| 28 | 3-(6,7-Dimethoxy-1- | H | NH₂ | H | H | OMe | OMe | N | CCH₃ |
| | methylisoquinolin-3-yl (aniline | | | | | | | | |
| | hydrochloride | | | | | | | | |
| 24 | 3-(3-Acetylphenyl)-6,7-dimethoxy-1- | H | COMe | H | H | OMe | OMe | N | CCH₃ |
| | methylisoquinolinium chloride | | | | | | | | |
| 25 | 3-(3-Acetylphenyl)-6,7-dimethoxy- | H | COMe | H | H | OMe | OMe | N⁺CH₃ | CCH₃ |
| | 1,2-dimethylisoquinolinium chloride | | | | | | | | |
| 29 | N-(3-(6,7-Dimethoxy-1- | H | N(SO₂Me)₂ | H | H | OMe | OMe | N | CCH₃ |
| | methylisoquinolin-3-yl)phenyl)-N- | | | | | | | | |
| | (methylsulfonyl)methanesulfonamide | | | | | | | | |
| | HCl | | | | | | | | |
| 31 | N-(3-(6,7-Dimethoxy-1- | H | NHCOMe | H | H | OMe | OMe | N | CCH₃ |
| | methylisoquinolin-3- | | | | | | | | |
| | yl)phenyl)acetamide hydrochloride | | | | | | | | |
| 30 | N-(3-(6,7-Dimethoxy-1- | H | NHSO₂Me | H | H | OMe | OMe | N | CCH₃ |
| | methylisoquinolin-3- | | | | | | | | |
| | yl)phenyl)methanesulfonamide HCl | | | | | | | | |
| 32 | Isopropyl 4-(6,7-dimethoxy-1- | H | H | COOiPr | H | OMe | OMe | N | CCH₃ |
| | methylisoquinolin-3-yl)benzoate HCl | | | | | | | | |
| 34 | 4-(6,7-Dimethoxy-1- | H | H | CONHMe | H | OMe | OMe | N | CCH₃ |
| | methylisoquinolin-3-yl)-N- | | | | | | | | |
| | methylbenzamide | | | | | | | | |
| 36 | 6,7-Dimethoxy-1-methyl-3-(pyridin-3- | H | H | H | H | OMe | OMe | N | CCH₃ |
| | yl)isoquinoline dihydrochloride | | | | | | | | |
| 37 | 2-(6,7-Dimethoxy-1- | NH₂ | H | H | H | OMe | OMe | N | CCH₃ |
| | methylisoquinolin-3-yl(aniline | | | | | | | | |
| | dihydrochloride | | | | | | | | |
| 38 | N-(2-(6,7-Dimethoxy-1- | NHCOMe | H | H | H | OMe | OMe | N | CCH₃ |
| | methylisoquinolin-3- | | | | | | | | |
| | yl)phenyl)acetamide | | | | | | | | |
| 39 | 3-(3,4-Dichlorophenyl)-6,7-dimethoxy- | H | Cl | Cl | H | OMe | OMe | N | CCH₃ |
| | 1-methylisoquinolinylium chloride | | | | | | | | |
| 40 | 6,7-Dimethoxy-3-(4-methoxyphenyl)- | H | H | OMe | H | OMe | OMe | N | CCH₃ |
| | 1-methylisoquinoline hydrochloride | | | | | | | | |
| 41 | 6,7-Dimethoxy-1-methyl-3- | H | —C═C—C═C— | | H | OMe | OMe | N | CCH₃ |
| | (naphthalen-2-yl)isoquinolinylium | | | | | | | | |
| | chloride | | | | | | | | |
| 42 | 3-(4-Chlorophenyl)-6,7-dimethoxy-1- | H | H | Cl | H | OMe | OMe | N | CCH₃ |
| | methylisoquinoline hydrochloride | | | | | | | | |
| 43 | 6,7-Dimethoxy-1-methyl-3-p- | H | H | Me | H | OMe | OMe | N | CCH₃ |
| | tolylisoquinoline hydrochloride | | | | | | | | |
| 44 | 6,7-Dimethoxy-1-methyl-3- | H | H | H | H | OMe | OMe | N | CCH₃ |
| | phenylisoquinoline hydrochloride | | | | | | | | |
| 35 | 6,7-Dimethoxy-3-(6-methoxypyridin-3- | H | H | OMe | H | OMe | OMe | N | CCH₃ |
| | yl)-1-methylisoquinoline | | | | | | | | |
| | dimethanesulfonate | | | | | | | | |
| 45 | 3-(3,4-dihydroxyphenyl)-6,7- | H | OH | OH | H | OH | OH | N⁺CH₃ | CCH₃ |
| | dihydroxy-1,2-dimethylisoquinolinium | | | | | | | | |
| | chloride | | | | | | | | |
| 47 | 4-(6,7-Dimethoxy-1- | H | OMe | OH | H | OMe | OMe | N | CCH₃ |
| | methylisoquinolin-3-yl)-2- | | | | | | | | |
| | methoxyphenol hydrochloride | | | | | | | | |
| 46 | 3-(2-(6,7-Dimethoxy-1- | NHCONMe₂ | H | H | H | OMe | OMe | N | CCH₃ |
| | methylisoquinolin-3-yl)phenyl)-1,1- | | | | | | | | |
| | dimethylurea HCl | | | | | | | | |
| 48 | 4-(6,7-dimethoxy-1-methylisoquinolin- | H | OH | OH | H | OH | OH | N | CCH₃ |
| | 3-yl)benzene-1,2-diol hydrochloride | | | | | | | | |

| # | D | E | F | G | J | Rac1 | Rac1b | Cdc42 |
|---|---|---|---|---|---|---|---|---|
| 50 | CH | C | — | — | C | 3 ± 5 | 10 ± 5 | 10 ± 6 |
| 49 | CH | C | — | — | C | 1 ± 5 | 2 ± 4 | 2 ± 6 |
| 21 | CH | C | — | — | C | 13 ± 2 | 12 ± 2 | 18 ± 2 |
| 22 | CH | C | — | — | C | 12 ± 2 | 18 ± 2 | 14 ± 3 |
| 23 | CH | C | — | — | C | 3 ± 4 | 10 ± 4 | 14 ± 3 |
| 26 | CH | C | — | — | C | 25 ± 3 | 46 ± 5 | 15 ± 1 |
| 27 | CH | C | — | — | C | 6 ± 2 | 17 ± 1 | 5 ± 2 |
| 28 | CH | C | — | — | C | 13 ± 2 | 20 ± 2 | 3 ± 5 |
| 24 | CH | C | — | — | C | 18 ± 3 | 27 ± 2 | 7 ± 2 |
| 25 | CH | C | — | — | C | 20 ± 3 | 29 ± 3 | 7 ± 1 |
| 29 | CH | C | — | — | C | −5 ± 2 | −2 ± 2 | 2 ± 4 |
| 31 | CH | C | — | — | C | −1 ± 2 | 10 ± 3 | 10 ± 1 |
| 30 | CH | C | — | — | C | 9 ± 3 | 16 ± 2 | 4 ± 4 |
| 32 | CH | C | — | — | C | 4 ± 5 | 10 ± 4 | 2 ± 1 |
| 34 | CH | C | — | — | C | −2 ± 3 | 3 ± 3 | 3 ± 5 |
| 36 | CH | C | — | — | N | 10 ± 3 | 12 ± 3 | −4 ± 13 |
| 37 | CH | C | — | — | C | 10 ± 1 | 14 ± 2 | 1 ± 13 |
| 38 | CH | C | — | — | C | 1 ± 3 | 0 ± 3 | 3 ± 7 |
| 39 | CH | C | — | — | C | 17 ± 7 | 42 ± 7 | 5 ± 1 |
| 40 | CH | C | — | — | C | 15 ± 3 | 17 ± 3 | 14 ± 1 |
| 41 | CH | C | — | — | C | 46 ± 2 | 48 ± 2 | 52 ± 4 |
| 42 | CH | C | — | — | C | 18 ± 3 | 17 ± 3 | 11 ± 2 |
| 43 | CH | C | — | — | C | 19 ± 1 | 17 ± 2 | 15 ± 2 |
| 44 | CH | C | — | — | C | 10 ± 3 | 8 ± 3 | 10 ± 1 |
| 35 | CH | C | — | — | N | 6 ± 4 | 6 ± 2 | 11 ± 2 |
| 45 | CH | C | — | — | C | 22 ± 1 | 36 ± 4 | 12 ± 1 |
| 47 | CH | C | — | — | C | 15 ± 3 | 18 ± 1 | 17 ± 1 |
| 46 | CH | C | — | — | C | 8 ± 3 | 9 ± 3 | 2 ± 0 |
| 48 | CH | C | — | — | C | 15 ± 1 | 28 ± 2 | 15 ± 3 |

ND: not determined All compounds were tested at a final concentration of 50 μM

TABLE 3
Benzo[c]phenanthridine Alkaloids

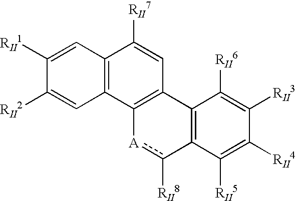

| Name | R_{II}¹ | R_{II}² | R_{II}³ | R_{II}⁴ | R_{II}⁵ | A | R_{II}⁶ | R_{II}⁷ | R_{II}⁸ | Rac1 | Rac1b | Cdc42 |
|---|---|---|---|---|---|---|---|---|---|---|---|---|
| Sanguinarine chloride hydrate 13 | OCH₂O | | H | OCH₂O | | N⁺Me | H | H | H | 45 ± 4 | 100 | 63 ± 2 |
| Chelerythrine chloride 14 | OCH₂O | | H | OMe | OMe | N⁺Me | H | H | H | 25 ± 6 | 100 | 35 ± 10 |
| 2,3-Dihydroxy-7,8-dimethoxy-5-methyl | OH | OH | H | OMe | OMe | N⁺Me | H | H | H | 47 ± 5 | 61 ± 4 | 38 ± 2 |
| benzo[c]phenanthridinium chloride 51 | | | | | | | | | | | | |
| 2,3,7,8-Tetrahydroxy-5-methyl | OH | OH | H | OH | OH | N⁺Me | H | H | H | 100 | 100 | 73 ± 4 |
| benzo[c]phenanthridinium chloride 52 | | | | | | | | | | | | |

All compounds were tested at a final concentration of 50 μM

1. An in vitro method for inhibiting a member of the Rho GTPase family, wherein the GTPase is contacted with at least one compound of formula (I) or (II), a compound of formula (I) having the following structure: 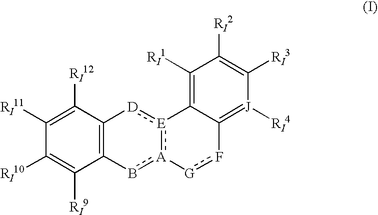
in which
J represents C or N;
R_{I}¹, R_{I}², R_{I}³ and R_{I}⁴ independently represent H, a halogen atom, a (C₁-C₆)alkyl group, an —OH group, an —O—(C₁-C₆)alkyl group, a (C₂-C₆)alkenyl group, a (C₂-C₆)alkynyl group, a —NO₂ group, a —NH₂ group, a —CO—(C₁-C₆)alkyl group preferably a —COCH₃ group, a —NH—SO₂—CH₃ group, a —N(SO₂CH₃)₂ group, a —NH—CO—CH₃ group, a NH—CO—N(CH₃)₂ group, a —COON group, a —COO(C₁-C₆)alkyl group preferably a —CO—O—CH(CH₃)₂ group, or a —CONH(C₁-C₆)alkyl group preferably a —CONHCH₃ group,
R_{I}⁴ being absent when J represents N and R_{I}⁴ being present when J represents C;
R_{I}⁹, R_{I}¹⁰ and R_{I}¹¹ independently represent H, an —OH group or an —O—(C₁-C₆)alkyl group;
or alternatively R_{I}² and R_{I}³ and/or R_{I}³ and R_{I}⁴ are fused together so as to form a naphthalene group or a quinolyl group with the adjacent cycle, or an —O—(CH₂)ₙ—O— group linked to the adjacent cycle, wherein n is an integer comprised between 1 and 6, and/or R_{I}⁹ and R_{I}¹⁰ and/or R_{I}¹⁰ and R_{I}¹¹ are fused together so as to form an —O—(CH₂)ₙ—O— group linked to the adjacent cycle, wherein n is an integer comprised between 1 and 6;
R_{I}¹² represents H, a C₁-C₆ alkyl group, a C₂-C₆ alkenyl group or a C₂-C₆ alkynyl group;
A represents N, N⁺, NH, N⁺H, N—(C₁-C₆)alkyl, N⁺(C₁-C₆)alkyl, N-arylalkyl preferably N-benzyl, or N⁺-arylalkyl preferably N⁺-benzyl;
B, absent or present, represents CH, CH₂, C-Methyl, C-Benzyl or C-Phenyl when B is present;
D, absent or present, represents CH or CH₂ when D is present;
E represents C, CH or CH₂;
G and F, absent or present, both represent either CH or CH₂ when present;
with the provisos that
at least one of B and D is present
both B and D are present when G and F are absent; and
when B or D is absent exclusively, then G and F are present;
its tautomers, optical and geometrical isomers, racemates, salts, hydrates and mixtures thereof;
and the compound of formula (II) having the following structure: 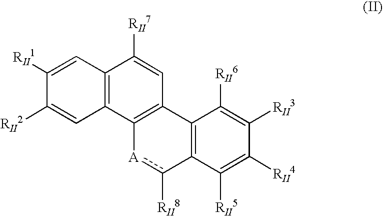
in which
R_{II}¹, R_{II}², R_{II}⁴ and R_{II}⁵ independently represent H, —OH or a —O(C₁-C₆)-alkyl group;
or alternatively wherein R_{II}¹ and R_{II}² and/or R_{II}⁴ and R_{II}⁵ are fused together so as to form an —O—(CH₂)ₙ—O— group linked to the adjacent cycle, wherein n is an integer comprised between 1 and 6;
R_{II}³, R_{II}⁶, R_{II}⁷ and R_{II}⁸ independently represent H, a (C₁-C₆)alkyl group, a (C₂-C₆)alkylene group or a (C₂-C₆)alkynyl group; and
A represents N, N⁺, N⁺(C₁-C₆)alkyl or N⁺-benzyl;
its tautomers, optical and geometrical isomers, racemates, salts, hydrates and mixtures thereof.

2. The method according to claim 1, for inhibiting Cdc42.

3. The method according to claim 1, for inhibiting a member of the Rac GTPase subfamily of Rho GTPase.

4. The method according to claim 3, for inhibiting Rac1 and/or Rac1b.

5. The method according to claim 1, wherein said compound of formula (I) is a compound of formula (I′) 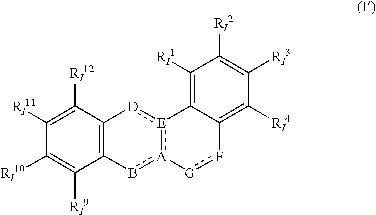
in which
R_{I}¹, R_{I}⁴ and R_{I}¹² independently represent H, a C₁-C₆ alkyl group, a C₂-C₆ alkenyl group or a C₂-C₆ alkynyl group;
R_{I}², R_{I}³, R_{I}⁹, R_{I}¹⁰ and R_{I}¹¹ independently represent H, —OH or an —O—(C₁-C₆)alkyl group;
or alternatively R_{I}² and R_{I}³ and/or R_{I}⁹ and R_{I}¹⁰ and/or R_{I}¹⁰ and R_{I}¹¹ are fused together so as to form an —O—(CH₂)ₙ—O— group linked to the adjacent cycle, wherein n is an integer comprised between 1 and 6;
A represents N, N⁺, N⁺(C₁-C₆)alkyl or N⁺-benzyl;
B, absent or present, B representing CH, CH₂, C-methyl, C-Benzyl or C-Phenyl when present;
D, absent or present, D representing CH or CH₂ when present;
with the proviso that at least one of B and D is present;
E represents C, CH or CH₂; and
F and G both represent either CH or CH₂;
its tautomers, optical and geometrical isomers, racemates, salts, hydrates and mixtures thereof.

6. The method according to claim 1, wherein R_{I}² and/or R_{I}³ represent —OH.

7. The method according to claim 1, wherein said compound is a compound of formula (I) in which R_{I}⁹ and R_{I}¹⁰ represent —OH.

8. The method according to claim 1, wherein said compound is a compound of formula (I) in which R_{I}² and R_{I}³ represent —OH, A is N⁺, B and D represent CH, E represents C and F and G both represent CH₂.

9. The method according to claim 1, wherein said compound of formula (I) is a compound of formula (V) 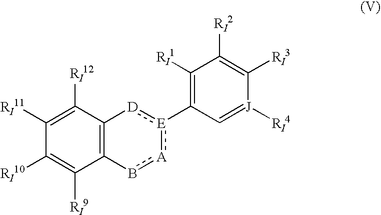
in which
J represents C or N;
R_{I}¹ represents H, a halogen atom, a (C₁-C₆)alkyl group, an —O—(C₁-C₆)alkyl group, a (C₂-C₆)alkenyl group, a (C₂-C₆)alkynyl group, a —NO₂ group, a —NH₂ group, a —CO—(C₁-C₆)alkyl group preferably a —COCH₃ group, a —NH—SO₂—CH₃ group, a —N(SO₂CH₃)₂ group, a —NH—CO—CH₃ group, a —NH—CO—N(CH₃)₂ group, a —COOH group, a —COO(C₁-C₆)alkyl group preferably a —CO—O—CH(CH₃)₂ group, or a —CONH(C₁-C₆)alkyl group preferably a —CONHCH₃ group;
R_{I}², R_{I}³ and R_{I}⁴ independently represent H, a halogen atom, a (C₁-C₆)alkyl group, an —OH group, an —O—(C₁-C₆)alkyl group, a (C₂-C₆)alkenyl group, a (C₂-C₆)alkynyl group, a —NO₂ group, a —NH₂ group, a —CO—(C₁-C₆)alkyl group preferably a —COCH₃ group, a —NH—SO₂—CH₃ group, a —N(SO₂CH₃)₂ group, a —NH—CO—CH₃ group, a NH—CO—N(CH₃)₂ group, a —COON group, a —COO(C₁-C₆)alkyl group preferably a —CO—O—CH(CH₃)₂ group, a —CONH(C₁-C₆)alkyl group preferably a —CONHCH₃ group;
R_{I}⁴ being absent when J represents N and R_{I}⁴ being present when J represents C;
R_{I}⁹, R_{I}¹⁰ and R_{I}¹¹ independently represent H or an —O—(C₁-C₆)alkyl group;
or alternatively R_{I}² and R_{I}³ or R_{I}³ and R_{I}⁴ are fused together so as to form a naphthalene group or a quinolyl group with the adjacent cycle, and/or R_{I}⁹ and R_{I}¹⁰ and/or R_{I}¹⁰ and R_{I}¹¹ are fused together so as to form an —O—(CH₂)ₙ—O— group linked to the adjacent cycle, wherein n is an integer comprised between 1 and 6;
R_{I}¹² represents H, a (C₁-C₆)alkyl group, a (C₂-C₆)alkenyl group or a (C₂-C₆)alkynyl group
A represents N, N⁺, NH, N⁺H, N—(C₁-C₆)alkyl, N⁺(C₁-C₆)alkyl, N-arylalkyl preferably N-benzyl or N⁺-arylalkyl preferably N⁺-benzyl;
B represents CH, CH₂, C-Methyl, C-Benzyl or C-Phenyl;
D represents CH or CH₂;
E represents C or CH;
at least one of R_{I}¹, R_{I}², R_{I}³ and R_{I}⁴ being different from a hydrogen atom when J represents C;
with the proviso that if one of R_{I}², R_{I}³ and R_{I}⁴ represents a —O(C₁-C₆)alkyl group, the other ones of R_{I}², R_{I}³ and R_{I}⁴ do not represent a —O(C₁-C₆)alkyl group;
its tautomers, optical and geometrical isomers, racemates, salts, hydrates and mixtures thereof.

10. The method according to claim 1, wherein said compound is a compound of formula (II) in which R_{II}¹ and R_{II}² and/or R_{II}⁴ and R_{II}⁵ are fused together so as to form an —O—(CH₂)ₙ—O— group linked to the adjacent cycle.

11. The method according to claim 1, wherein said compound is selected from the group consisting of:
Berberine or 1,2-dimethoxy-N-methyl-[1,3]benzodioxolo[5,6-c]phenanthridinium chloride 1,
palmatine chloride, hydrate 2,
(±)-canadine or (±)-tetrahydroberberine hydrochloride 3,
demethyleneberberine or 9,10-dimethoxy-5,6-dihydro-isoquino[3,2-a]isoquinolinylium-2,3-diol chloride 4,
(±)-N-benzyl canadinium or (±)-7-benzyl-9,10-dimethoxy-5,8,13,13a-tetrahydro-6H-[1,3]dioxolo[4,5-g]isoquino[3,2-a]isoquinolinylium bromide 5,
2,3,9,10-tetrahydroxyberberine or 5,6-dihydro-isoquino[3,2-a]isoquinolinylium-2,3,9,10-tetraol chloride 6,
2-(2,3-dimethoxybenzyl)-6,7-dimethoxy-1,2,3,4-tetrahydroisoquinoline hydrochloride 7,
coralyne or 8-methyl-2,3,10,11-tetramethoxydibenzo[a,g]quinolizinium chloride, hydrate 8,
papaverine or 1-(3,4-dimethoxybenzyl)-6,7-dimethoxyisoquinoline hydrochloride 9,
9,10-dimethoxy-8-phenyl-5,8-dihydro-2H-6H-[1,3]dioxolo[4,5-g]isoquino[3,2-a]isoquinoline 10,
8-benzyl-9,10-dimethoxy-5,8-dihydro-2H-6H-[1,3]dioxolo[4,5-g]isoquino[3,2-a]isoquinoline 11,
(±)-8-benzyl-9,10-dimethoxy-5,8,13,13a-tetrahydro-6H-[1,3]dioxolo[4,5-g]isoquino[3,2-a]isoquinoline hydrochloride 12,
Sanguinarine or 13-methyl-[1,3]benzodioxolo[5,6-c]-1,3-dioxolo[4,5-i]phenanthridinium chloride hydrate 13,
chelerythrine or 1,2-dimethoxy-N-methyl[1,3]benzodioxolo[5,6-c]phenanthridinium chloride 14,
8-methyl-isoquino[3,2-a]isoquinolinylium-2,3,10,11-tetraol chloride 15,
(±)-tetrahydroxytetrahydroberberine or (±)-5,8,13,13a-tetrahydro-6H-isoquino[3,2-a]isoquinolinylium-2,3,9,10-tetraol hydrochloride 16.
(±)-9,10-Dimethoxy-5,8,13,13a-tetrahydro-6H-isoquino[3,2-a]isoquinoline-2,3-diol hydrochloride 17,
2-(2,3-Dihydroxybenzyl)-6,7-dimethoxy-1,2,3,4-tetrahydroisoquinolinium chloride 18,
2-(2,3-Dihydroxybenzyl)-6,7-dihydroxy-1,2,3,4-tetrahydroisoquinolinium chloride 19,
(±)-3-(6-Ethylbenzo[d][1,3]dioxol-5-yl)-7,8-dimethoxy-2-methyl-1,2,3,4-tetrahydroisoquinolinylium chloride 20.
3-(Benzo[d][1,3]dioxol-5-yl)-6,7-dimethoxy-1-methylisoquinoline hydrochloride 21,
3-(Benzo[d][1,3]dioxol-5-yl)-6,7-dimethoxy-1,2-dimethylisoquinolinium chloride 22,
6,7-Dimethoxy-1-methyl-3-(3-nitrophenyl)isoquinolinium chloride 23,
1-(3-(6,7-Dimethoxy-1-methylisoquinolin-3-yl)phenyl)ethanone hydrochloride 24,
3-(3-Acetylphenyl)-6,7-dimethoxy-1,2-dimethylisoquinolinium chloride 25,
4-(6,7-Dimethoxy-1-methylisoquinolin-3-yl)benzene-1,2-diol hydrochloride 26,
3-(3,4-Dihydroxyphenyl)-6,7-dimethoxy-1,2-dimethylisoquinolinium chloride 27,
3-(6,7-Dimethoxy-1-methylisoquinolin-3-yl)aniline dihydrochloride 28,
N-(3-(6,7-Dimethoxy-1-methylisoquinolin-3-yl)phenyl)-N-(methylsulfonyl)methanesulfonamide hydrochloride 29,
N-(3-(6,7-Dimethoxy-1-methylisoquinolin-3-yl)phenyl)methanesulfonamide hydrochloride 30,
N-(3-(6,7-Dimethoxy-1-methylisoquinolin-3-yl)phenyl)acetamide hydrochloride 31,
Isopropyl 4-(6,7-dimethoxy-1-methylisoquinolin-3-yl)benzoate hydrochloride 32,
4-(6,7-Dimethoxy-1-methylisoquinolin-3-yl)benzoic acid hydrochloride 33,
4-(6,7-Dimethoxy-1-methylisoquinolin-3-yl)-N-methylbenzamide 34,
6,7-Dimethoxy-3-(6-methoxypyridin-3-yl)-1-methylisoquinoline dimethanesulfonate 35,
6,7-Dimethoxy-1-methyl-3-(pyridin-3-yl)isoquinoline dihydrochloride 36,
2-(6,7-Dimethoxy-1-methylisoquinolin-3-yl)aniline dihydrochloride 37,
N-(2-(6,7-Dimethoxy-1-methylisoquinolin-3-yl)phenyl)acetamide 38,
3-(3,4-Dichlorophenyl)-6,7-dimethoxy-1-methylisoquinolinylium chloride 39,
6,7-Dimethoxy-3-(4-methoxyphenyl)-1-methylisoquinolinylium chloride 40,
6,7-Dimethoxy-1-methyl-3-(naphthalen-2-yl)isoquinolinylium chloride 41,
3-(4-Chlorophenyl)-6,7-dimethoxy-1-methylisoquinolinylium chloride 42,
6,7-Dimethoxy-1-methyl-3-p-tolylisoquinolinylium chloride 43,
6,7-Dimethoxy-1-methyl-3-phenylisoquinolinylium chloride 44,
3-(3,4-Dihydroxyphenyl)-6,7-dihydroxy-1,2-dimethylisoquinolinium chloride 45,
3-(2-(6,7-Dimethoxy-1-methylisoquinolin-3-yl)phenyl)-1,1-dimethylurea hydrochloride 46,
4-(6,7-Dimethoxy-1-methylisoquinolin-3-yl)-2-methoxyphenol hydrochloride 47,
4-(6,7-dimethoxy-1-methylisoquinolin-3-yl)benzene-1,2-diol hydrochloride 48,
6,7-Dimethoxy-3-phenylisoquinolinium chloride 49,
6,7-Dimethoxy-2-methyl-3-phenylisoquinolinium chloride 50,
2,3-Dihydroxy-7,8-dimethoxy-5-methylbenzo[c]phenanthridinium chloride 51,
2,3,7,8-Tetrahydroxy-5-methylbenzo[c]phenanthridinium chloride 52.

12. The method according to claim 1, wherein said member of the Rho GTPase family is Rac1b and said compound is a compound of formula (II).

13. The method according to claim 12, wherein said compound is selected from the group consisting of:
Sanguinarine or 13-methyl-[1,3]benzodioxolo[5,6-c]-1,3-dioxolo[4,5-i]phenanthridinium chloride hydrate 13,
chelerythrine or 1,2-dimethoxy-N-methyl[1,3]benzodioxolo[5,6-c]phenanthridinium chloride 14, and
2,3,7,8-tetrahydroxy-5-methylbenzo[c]phenanthridinium chloride 52.

14. A compound of formula (V) 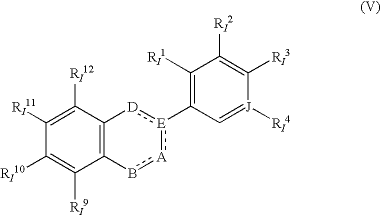
in which
J represents C or N;
R_{I}¹ represents H, a halogen atom, a (C₁-C₆)alkyl group, an —O—(C₁-C₆)alkyl group, a (C₂-C₆)alkenyl group, a (C₂-C₆)alkynyl group, a —NO₂ group, a —NH₂ group, a —CO—(C₁-C₆)alkyl group preferably a —COCH₃ group, a —NH—SO₂—CH₃ group, a —N(SO₂CH₃)₂ group, a —NH—CO—CH₃ group, a —NH—CO—N(CH₃)₂ group, a —COOH group, a —COO(C₁-C₆)alkyl group preferably a —CO—O—CH(CH₃)₂ group, or a —CONH(C₁-C₆)alkyl group preferably a —CONHCH₃ group;
R_{I}², R_{I}³ and R_{I}⁴ independently represent H, a halogen atom, a (C₁-C₆)alkyl group, an —OH group, an —O—(C₁-C₆)alkyl group, a (C₂-C₆)alkenyl group, a (C₂-C₆)alkynyl group, a —NO₂ group, a —NH₂ group, a —CO—(C₁-C₆)alkyl group preferably a —COCH₃ group, a —NH—SO₂—CH₃ group, a —N(SO₂CH₃)₂ group, a —NH—CO—CH₃ group, a NH—CO—N(CH₃)₂ group, a —COOH group, a —COO(C₁-C₆)alkyl group preferably a —CO—O—CH(CH₃)₂ group, a —CONH(C₁-C₆)alkyl group preferably a —CONHCH₃ group;
R_{I}⁴ being absent when J represents N and R_{I}⁴ being present when J represents C;
R_{I}⁹, R_{I}¹⁰ and R_{I}¹¹ independently represent H or an —O—(C₁-C₆)alkyl group;
or alternatively R_{I}² and R_{I}³ or R_{I}³ and R_{I}⁴ are fused together so as to form a naphthalene group or a quinolyl group with the adjacent cycle, and/or R_{I}⁹ and R_{I}¹⁰ and/or R_{I}¹⁰ and R_{I}¹¹ are fused together so as to form an —O—(CH₂)ₙ—O— group linked to the adjacent cycle, wherein n is an integer comprised between 1 and 6;
R_{I}¹² represents H, a (C₁-C₆)alkyl group, a (C₂-C₆)alkenyl group or a (C₂-C₆)alkynyl group
A represents N, N⁺, NH, N⁺H, N—(C₁-C₆)alkyl, N⁺(C₁-C₆)alkyl, N-arylalkyl preferably N-benzyl or N⁺-arylalkyl preferably N⁺-benzyl;
B represents CH, CH₂, C-Methyl, C-Benzyl or C-Phenyl;
D represents CH or CH₂;
E represents C or CH;
at least one of R_{I}¹, R_{I}², R_{I}³ and R_{I}⁴ being different from a hydrogen atom when J represents C;
with the proviso that if one of R_{I}², R_{I}³ and R_{I}⁴ represents a —O(C₁-C₆)alkyl group, the other ones of R_{I}², R_{I}³ and R_{I}⁴ do not represent a —O(C₁-C₆)alkyl group;
its tautomers, optical and geometrical isomers, racemates, salts, hydrates and mixtures thereof.

15. A compound according to claim 14, wherein J represents C.

16. A compound according to claim 14, wherein R_{I}⁹ represents H and R_{I}¹⁰ and R_{I}¹¹ both represent a —O(C₁-C₆)alkyl group, preferably a —O—CH₃ group.

17. A compound according to claim 14, wherein at least one of R_{I}¹, R_{I}², R_{I}³ and R_{I}⁴ represents a —NO₂ group, a —NH₂ group, a —NH—SO₂—CH₃ group, a —N(SO₂CH₃)₂ group, a —NH—CO—CH₃ group or a NH—CO—N(CH₃)₂ group.

18. A compound according to claim 14, wherein at least one of R_{I}¹, R_{I}², R_{I}³ and R_{I}⁴ represents a —CO—(C₁-C₆)alkyl group, preferably a —COCH₃, a —COON group, a —COO(C₁-C₆)alkyl group, preferably a —COOCH(CH₃)₂ group, or a —CONH(C₁-C₆)alkyl group, preferably a —CONHCH₃ group.

19. A compound according to claim 14 wherein at least one of R_{I}¹, R_{I}², R_{I}³ and R_{I}⁴ represents an —OH group, an —O—(C₁-C₆)alkyl group or a (C₁-C₆)alkyl group.

20. A compound according to claim 14, wherein J represents C, R_{I}⁴ represents a hydrogen atom and R_{I}² and R_{I}³ are fused together so as to form a naphthalene group.

21. A compound according to claim 14, wherein at least one of R_{I}¹, R_{I}², R_{I}³ and R_{I}⁴ represents a halogen atom.

22. The compound according to claim 14, wherein said compound is selected in the group consisting of
6,7-Dimethoxy-1-methyl-3-(3-nitrophenyl)isoquinolinium chloride 23,
1-(3-(6,7-Dimethoxy-1-methylisoquinolin-3-yl)phenyl)ethanone hydrochloride 24,
3-(3-Acetylphenyl)-6,7-dimethoxy-1,2-dimethylisoquinolinium chloride 25,
4-(6,7-Dimethoxy-1-methylisoquinolin-3-yl)benzene-1,2-diol hydrochloride 26,
3-(3,4-Dihydroxyphenyl)-6,7-dimethoxy-1,2-dimethylisoquinolinium chloride 27,
3-(6,7-Dimethoxy-1-methylisoquinolin-3-yl)aniline dihydrochloride 28,
N-(3-(6,7-Dimethoxy-1-methylisoquinolin-3-yl)phenyl)-N-(methylsulfonyl)methanesulfonamide hydrochloride 29,
N-(3-(6,7-Dimethoxy-1-methylisoquinolin-3-yl)phenyl)methanesulfonamide hydrochloride 30,
N-(3-(6,7-Dimethoxy-1-methylisoquinolin-3-yl)phenyl)acetamide hydrochloride 31,
Isopropyl 4-(6,7-dimethoxy-1-methylisoquinolin-3-yl)benzoate hydrochloride 32,
4-(6,7-Dimethoxy-1-methylisoquinolin-3-yl)benzoic acid hydrochloride 33,
4-(6,7-Dimethoxy-1-methylisoquinolin-3-yl)-N-methylbenzamide 34,
6,7-Dimethoxy-3-(6-methoxypyridin-3-yl)-1-methylisoquinoline dimethanesulfonate 35,
6,7-Dimethoxy-1-methyl-3-(pyridin-3-yl)isoquinoline dihydrochloride 36,
2-(6,7-Dimethoxy-1-methylisoquinolin-3-yl)aniline dihydrochloride 37,
N-(2-(6,7-Dimethoxy-1-methylisoquinolin-3-yl)phenyl)acetamide 38,
3-(3,4-Dichlorophenyl)-6,7-dimethoxy-1-methylisoquinolinylium chloride 39,
6,7-Dimethoxy-3-(4-methoxyphenyl)-1-methylisoquinolinylium chloride 40,
6,7-Dimethoxy-1-methyl-3-(naphthalen-2-yl)isoquinolinylium chloride 41,
3-(4-Chlorophenyl)-6,7-dimethoxy-1-methylisoquinolinylium chloride 42,
6,7-Dimethoxy-1-methyl-3-p-tolylisoquinolinylium chloride 43,
3-(2-(6,7-Dimethoxy-1-methylisoquinolin-3-yl)phenyl)-1,1-dimethylurea hydrochloride 46, and
4-(6,7-Dimethoxy-1-methylisoquinolin-3-yl)-2-methoxyphenol hydrochloride 47.

23. A compound of formula (II) as defined in claim 1, wherein said compound is selected in the group consisting of:
2,3-Dihydroxy-7,8-dimethoxy-5-methylbenzo[c]phenanthridinium chloride 51, and
2,3,7,8-Tetrahydroxy-5-methylbenzo[c]phenanthridinium chloride 52.

24. A pharmaceutical composition comprising at least one compound according to claim 14 and a pharmaceutically acceptable vehicle or support.

25. A compound selected in the group consisting of 2,3-dihydroxy-7,8-dimethoxy-5-methylbenzo[c]phenanthridinium chloride 51 and 2,3,7,8-tetrahydroxy-5-methylbenzo[c]phenanthridinium chloride 52 for the treatment of cancer.
